SEQUENCE LISTING (3 sequences)

SEQ ID NO: 1 - DNA, 3802 bp, synthetic construct
  source 1..3802: mol_type = other DNA; note = pSV40-hBAX-BLa
tcgcgatgtacgggccagatatacgcgttctgtggaatgtgtgtcagttagggtgtggaaagtccccaggctccccagcaggcagaagtatgcaaagcatgcatctcaattagtcagcaaggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaagaggcttttttggaggcctaggcttttgcaaaaagctccggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcgcggtgatggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggcctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcactcaccatctggaagaagatgggctgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccaccggtacccgggcccatggcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtactagtactcgagctcgcgaagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgct

SEQ ID NO: 2 - DNA, 4880 bp, synthetic construct
  source 1..4880: mol_type = other DNA; note = pSV40-sGAD55-BLa
tcgcgatgtacgggccagatatacgcgttctgtggaatgtgtgtcagttagggtgtggaaagtccccaggctccccagcaggcagaagtatgcaaagcatgcatctcaattagtcagcaaggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaagaggcttttttggaggcctaggcttttgcaaaaagctccggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccactagtccagtgtggtggaattctgcagatatccagcacagtggcggccgctcgacggtatcgataagcttgatatcgaattcgttgggattttctagaatgtacaggatgcaactcctgtcttgcattgcactaagtcttgcacttgtcacaaacagtgcacctacttacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcgtccgcatggtcatctcaaacccagcggcaactcaccaagacattgacttcctgattgaagaaatagaacgccttggacaagatttataataaccttgctcaccaagctgttcacttcttcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccaccggtacccgggcccatggcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtactagtactcgagctcgcgaagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgct

SEQ ID NO: 3 - DNA, 6419 bp, synthetic construct
  source 1..6419: mol_type = other DNA; note = pSV40-sGAD55+hBAX-BLa
tcgcgatgtacgggccagatatacgcgttctgtggaatgtgtgtcagttagggtgtggaaagtccccaggctccccagcaggcagaagtatgcaaagcatgcatctcaattagtcagcaaggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaagaggcttttttggaggcctaggcttttgcaaaaagctccggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattcggatccactagtccagtgtggtggaattctgcagatatccagcacagtggcggccgctcgacggtatcgataagcttgatatcgaattcgttgggattttctagaatgtacaggatgcaactcctgtcttgcattgcactaagtcttgcacttgtccacctacttacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcgtccgcatggtcatctcaaacccagcggcaactcaccaagacattgacttcctgattgaagaaatagaacgccttggacaagatttataataaccttgctcaccaagctgttcacttctagaatcactagtgcggccgcctgcaggtcgaggatccagaggtaccgagctcgaattctgcagatatccatcacactggcggccgcctgcaggtcgaggatccagaggtaccgagctcgaattctgcagatatccatcacactggcggccgcctgcaggtcgaggatccagaggtaccgagctcgaattctgcagatatccatcacactggcggccgcctgcaggtcgaggatccagaggtaccgagctcgaattctgcagatatccatcacactggcggccgctctagaactagtggatcccccgggctgcaggaattcgataaacctagaaacacgactcactatagggcgaattccgcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgattattatattgcctctaggatggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggcctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcactcaccatctggaagaagatgggctgagaattctgcagatatatcaagcttatcgataccgtcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccaccggtacccgggcccatggcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtactagtactcgagctcgcgaagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttgctgct

(19) United States
(12) Patent Application Publication
(10) Pub. No.: US 2024/0352469 A1
(43) Pub. Date: Oct. 24, 2024

(54) ENZYMATICALLY METHYLATED DNA AND METHODS OF PRODUCTION AND THERAPEUTIC USE

(71) Applicant: ADITXT, INC., Mountain View, CA (US)

(72) Inventors: Marco PALIZA-CARRE, Mountain View, CA (US); Shahrokh SHABAHANG, Mountain View, CA (US); Ge CHEN, Mountain View, CA (US)

(21) Appl. No.: 18/687,772

(22) PCT Filed: Aug. 26, 2022
(86) PCT No.: PCT/US2022/075521

§ 371 Date: Feb. 28, 2024

Related U.S. Application Data
(60) Provisional application No. 63/238,726, filed on Aug. 30, 2021.
(60) Provisional application No. 63/240,341, filed on Sep. 2, 2021.
(60) Provisional application No. 63/247,714, filed on Sep. 23, 2021.

(51) Int. Cl. C12N 15/117; A61K 39/00

(52) U.S. Cl. CPC C12N 15/117; A61K 39/0008; A61K 39/001; A61K 39/0011; A61K 2039/53

(57) ABSTRACT

The present disclosure provides a method of enzymatically methylating a DNA by combining a source DNA encoding a pro-apoptotic protein, a determinant protein, or a functional fragment thereof, an extracellular methylation enzyme and an enzymatic substrate in an amount sufficient to allow methylation of at least one CpG site on the source DNA and then incubating the reaction sample at a temperature and for a time sufficient to obtain an enzymatically methylated DNA having a specific methylation level or a specific methylation pattern. The disclosure further provides enzymatically methylated DNA, pharmaceutical compositions containing enzymatically methylated DNA, and methods of treatment using such enzymatically methylated DNA or pharmaceutical compositions.

### TECHNICAL FIELD

The present disclosure relates generally to enzymatically methylated DNA and compositions containing enzymatically methylated DNA, particularly with a methylation level or methylation pattern that allows targeted modulation of the immune response in a patient. The present disclosure further provides methods of enzymatically methylating DNA, particularly to a target methylation level or in a target methylation pattern, and methods of using enzymatically methylated DNA to modulate the immune response in a patient.

### BACKGROUND

Many patients are benefitted by modulation of their immune response. Current therapies for many of these diseases rely on non-specific modulation of the immune response, which may cause increased side effects or even prevent use of the therapy in some patients all together.

For example, downregulation of the immune response in transplant patients can avoid rejection of transplanted organs and tissues. Similarly, downregulation of the immune response in some autoimmune disease patients may lessen symptoms of the disease. However, most current therapies cause generalized immune suppression, which can lead to potentially serious infections.

As another example, upregulation of the immune response in cancer patients may allow their bodies to attack cancer cells previously overlooked by the immune system. However, this same upregulation can cause the patients to develop autoimmune diseases or to otherwise develop serious complications of general immune activation, such as severe inflammation.

More recently, methods of specifically regulating the immune response have been developed. In these methods, the patient is treated with enzymatically methylated DNA to regulate the immune response in a more specific manner. The immune response may be upregulated or downregulated, depending on the methylation level of the DNA. DNA for use in these treatments has been methylated using bacteria expressing a methyltransferase gene. However, bacterial methylation is temperature-sensitive, requires subsequent purification to remove bacterial components, and exhibits batch-to-batch variability in methylation that is somewhat high.

### BRIEF SUMMARY

Methods of the present disclosure may provide highly controllable and reproducible methylation results in vitro. Methylation levels and patterns of enzymatically methylated DNA may be manipulated over a wide range by varying enzymatic methylation parameters, such as incubation time, enzyme concentration, source DNA topology, and magnesium concentration. Enzymatic methylation methods of the present disclosure may be used to obtain DNA samples that display digestion patterns similar to those obtained using bacterial methylation, indicating the suitability of enzymatically methylated DNA for use in place of bacterially methylated DNA.

The present disclosure provides a method of enzymatically methylating a DNA by combining a source DNA encoding a pro-apoptotic protein, a determinant protein, or a functional fragment thereof, an extracellular methylation enzyme and an enzymatic substrate in an amount sufficient to allow methylation of at least one CpG site on the source DNA and then incubating the reaction sample at a temperature and for a time sufficient to obtain an enzymatically methylated DNA having a specific methylation level or a specific methylation pattern.

In more specific embodiments:
the methylation level is a CpG site specific methylation level, a mean whole DNA methylation level, or a fractional methylation level;
the source DNA has a mean whole DNA methylation level of less than about 3%, such as about 0%;
the source DNA may be produced in a host cell that has a methyltransferase gene;
the source DNA may be produced in a host cell that lacks a methyltransferase gene;
the source DNA may be artificially synthesized;
the source DNA may be linear;
the source DNA may be a covalently closed linear double-stranded DNA;
the source DNA may be a plasmid;
the source DNA may be a covalently closed circular double-stranded DNA;
the method may further include a second methylation enzyme;
at least one methylation enzyme may be a DNA methyltransferase;
at least one DNA methyltransferase may be a bacterial DNA methyltransferase, such as a M.SssI methyltransferase or a M.MpeI methyltransferase, an AluI methyltransferase, a HaeIII methyltransferase, a HhaI methyltransferase, a HpaII methyltransferase, a MspI methyltransferase, a BamHI methyltransferase, a Dam methyltransferase, an EcoGII methyltransferase, an EcoRI methyltransferase, a GpC methyltransferase (M.CviPI), a MspI methyltransferase, or a TaqI methyltransferase;
at least one DNA methyltransferase may be DNMT1, DNMT2, DNMT3a, or DNMT3b;
at least one methylation enzyme may be present in the reaction sample in an amount of at least about 0.1U or in a range from about 1U to about 5U;
the enzymatic substrate may be S-adenosyl methionine (SAM), for example at least one methylation enzyme may be M.SssI and the SAM may be present in the reaction sample in a concentration in a range from about 150 μM to about 200 μM;
incubation may be for a period of time in a range from about 1 minute to about 24 hours, for example, at least one methylation enzyme may be M.SssI and the period of time may be in a range from about 15 minutes to about 1 hour;
the incubation may be at a temperature in a range from about 25° C. to about 45° C., for example, at least one methylation enzyme may be M.SssI and the incubation may be at a temperature in a range from about 25° C. to about 37° C.;
the incubation may be at a temperature in a range from about 0° C. to about 100° C., and at least one methylation enzyme is a low-temperature or high-temperature active enzyme;
the incubation may be at a temperature in a range from about 25° C. to about 100° C., and at least one methylation enzyme is a high-temperature active enzyme;
the method may further include combining magnesium ion in the reaction sample, for example at least one methylation enzyme may be M. SssI and the magnesium ion may be at a concentration in a range from about 1 mM to about 200 mM;
the method may further include treating the source DNA prior to combining to remove one or more associated material that affects DNA accessibility or to cause a conformational change of the source DNA, for example treating the source DNA may remove associated proteins or eliminate supercoiling;
the method may further include, after incubating, raising the temperature of the reaction sample to a quenching temperature of at least 50° C.;
the method may further include treating the enzymatically methylated DNA to alter its structure;
the enzymatically methylated DNA may have a CpG site specific stimulating methylation level at least one CpG site;
the enzymatically methylated DNA may have a CpG site specific attenuating methylation level of at least one CpG site;
the enzymatically methylated DNA may have a mean whole DNA stimulating methylation level;
the enzymatically methylated DNA may have a mean whole DNA attenuating methylation level;
the enzymatically methylated DNA may have a stimulating fractional methylation level;
the enzymatically methylated DNA may have an attenuating fractional methylation level;
the enzymatically methylated DNA may be pSV40-sGAD55, pSV40-hBAX-BLa, or pSV40-sGAD55+hBAX-BLa;
the determinant protein may be an allergen, an autoantigen, a cancer antigen, a donor antigen, a sequestered tissue specific antigen, or a functional fragment thereof;
the source DNA may further encode a tolerance inducing protein or a functional fragment thereof;
the method may further include demethylating the source DNA prior to combining the source DNA with the extracellular methylation enzyme; and/or
the methylation enzyme may include comprises a CpG specific methylation enzyme and the enzymatically methylated DNA may be methylated at at least one CpG site.

The present disclosure also provides an enzymatically methylated DNA prepared according to any of the above methods or any other methods as disclosed herein.

The present disclosure further provides an enzymatically methylated DNA encoding a pro-apoptotic protein or a functional fragment thereof and having a stimulating methylation level.

Either enzymatically methylated DNA may include a pro-apoptotic protein that may be BCL2 associated X protein (BAX), BCL2-antagonist/killer 1 (Bak), BCL-2-interacting mediator of cell death (Bim), p53 up-regulated modulator of apoptosis (Puma), BCL-2 associated agonist of cell death (Bad), BCL-2-interacting killer (Bik), phorbol-12-myristate-13-acetate-induced protein 1 (Noxa), BCL-2 modifying factor (Bmf), hara-kiri (Hrk), BH3 interacting-domain death agonist (Bid), FAS, a FAS receptor, second mitochondria-derived activator of caspase (Smac), HtrA serine peptidase 2 (Omi/HtrA2), Septin 4 (ARTS/Sep4), Death Receptor 4 (DR4), Death Receptor 5 (DRS), apoptosis inducing factor (AIF), cytochrome C, endonuclease G, Caspase-activated deoxyribonuclease (CAD), apoptosis protease activating factor-1 (APAF-1) a Tumor Necrosis Factor Receptor, an apoptosis-inducing caspase mutant, an apoptosis-inducing modified caspase, an apoptosis-inducing survivin mutant, an apoptosis-inducing modified survivin, an apoptosis-inducing IAP mutant, or IAP antagonist.

Any enzymatically methylated DNA above may further encode a cancer antigen and having a stimulating methylation level. The cancer antigen may be a Burkitt lymphoma, neuroblastoma, melanoma, osteosarcoma, renal cell carcinoma, breast cancer, prostate cancer, lung carcinoma, colon cancer, germ cell tumors, ovarian cancer, or hepatocellular carcinoma cancer antigen. The cancer antigen may also be alphafetoprotein (AFP), carcinoembryonic antigen (CEA), cancer antigen-125 (CA-125), mucin 1, cell surface associated (MUC1), epithelial tumor antigen (ETA), tyrisonase, melanoma-associated antigen (MAGE), or a mutant of ras or p53, WT1, mesothelin, KRAS, ROR1, EGFR, EGFRVIII, EGP-2, EGP-40, GD2, GD3, HPV E6, HPV E7, Her2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, NY-ESO, 5T4, BCMA, FAP, Carbonic anhydrase 9, BRAF, α-fetoprotein, MAGE-A3, MAGE-A4, SSX-2, PRAME, HA-1, B2M, ETA, tyrosinase, NRAS, or CEA antigen.

The present disclosure also provides an enzymatically methylated DNA encoding an allergen, an autoantigen, a donor antigen, a sequestered tissue specific antigen, or a functional fragment thereof and having an attenuating methylation level. In more specific embodiments, the allergen may be a pollen protein, an animal dander protein, a dust mite protein, an insect protein, a protein-based medication, a, mold protein, or a food protein, or a hapten that causes an allergic response once combined with host cellular elements. In other specific embodiments, the autoantigen may be a carbonic anhydrase II, chromogranin, collagen, CYP2D6 (cytochrome P450, family 2, subfamily Device 400, polypeptide 6), glutamic acid decarboxylase (GAD), secreted glutamic acid decarboxylase 55 (sGAD), islet cell antigen 512 (IA2), islet-specific glucose-6-phosphatase catalytic subunit-related protein (IGRP), insulin, myelin basic protein, human Ninein (hNinein), Ro 60 kDa, SRY-box containing gene 10 (SOX-10), zinc transporter 8 (ZnT8), thyroglobulin, thyroperoxidase, thyroid stimulating hormone receptor, chromogranin A (ChgA), islet amyloid polypeptide (IAPP), peripherin, tetraspanin-7, prolyl-4-hydroxylase β (P4Hb), glucose-regulated protein 78 (GRP78), urocortin-3, insulin gene enhancer protein isl-1, 21OH hydroxylase, 17OH hydroxylase, H+/K+ ATPase, transglutaminase, tyrosinase, tyrosinase-related protein-2, myelin basic protein, proteolipid protein, desmogleins, hepatocyte antigens, cytochrome; P450-1A2, acetylcholine receptor, 2-oxoacid dehydrogenase complexes, trichohyalin, cathelicidin LL-37, melanocytic ADAMTSL5, lipid antigen PLA2G4D, or keratin 17. In some embodiments, the enzymatically methylated DNA may further encode a pro-apoptotic protein or a functional fragment thereof, which may be BCL2 associated X protein (Bax), BCL2-antagonist/killer 1 (Bak), BCL-2-interacting mediator of cell death (Bim), p53 up-regulated modulator of apoptosis (Puma), BCL-2 associated agonist of cell death (Bad), BCL-2-interacting killer (Bik), phorbol-12-myristate-13-acetate-induced protein 1 (Noxa), BCL-2 modifying factor (Bmf), hara-kiri (Hrk), BH3 interacting-domain death agonist (Bid), FAS, a FAS receptor, second mitochondria-derived activator of caspase (Smac), HtrA serine peptidase 2 (Omi/HtrA2), Septin 4 (ARTS/Sep4), Death Receptor 4 (DR4), Death Receptor 5 (DRS), apoptosis inducing factor (AIF), cytochrome C, endonuclease G, Caspase-activated deoxyribonuclease (CAD), apoptosis protease activating factor-1 (APAF-1) a Tumor Necrosis Factor Receptor, an apoptosis-inducing caspase mutant, an apoptosis-inducing modified caspase, an apoptosis-inducing survivin mutant, an apoptosis-inducing modified survivin, an apoptosis-inducing IAP mutant, or IAP antagonist. In some more specific embodiments, the enzymatically methylated DNA may encode a pro-apoptotic protein, an allergen, an autoantigen, or a donor antigen, a sequestered tissue specific antigen, or a functional fragment thereof and a tolerance-inducing protein or a functional fragment thereof. For example, enzymatically methylated DNA may be pSV40-sGAD55, pSV40-hBAX-BLa, or pSV40-sGAD55+hBAX-BLa.

The present disclosure further provides a pharmaceutical composition including an enzymatically methylated DNA as set forth above or elsewhere in the present disclosure and a pharmaceutically acceptable carrier. The enzymatically methylated DNA may be coupled to a delivery vehicle or carrier, which may include a lipid or lipid-derived delivery vehicle, a nanoscale platform, or a viral-based carrier. The pharmaceutical composition may be a liquid pharmaceutical composition. The pharmaceutical composition may include two different enzymatically methylated DNAs as set forth above or elsewhere in the present disclosure.

In one embodiment, the pharmaceutical composition may include a first enzymatically methylated DNA that includes a gene encoding pro-apoptotic protein or a functional fragment thereof and a second enzymatically methylated DNA that includes s a gene encoding an allergen, an autoantigen, a donor antigen, a sequestered tissue specific antigen, or a pro-apoptotic protein. More specifically, the first enzymatically methylated DNA may have a stimulating methylation level and the second enzymatically methylated DNA may have an attenuating methylation level.

The present disclosure further provides a method of treating an allergy in a patient by administering to the patient a therapeutically effective amount of an enzymatically methylated DNA or a pharmaceutical composition as described above or elsewhere in the present disclosure.

The present disclosure further provides a method of treating an autoimmune disease in a patient by administering to the patient a therapeutically effective amount of an enzymatically methylated DNA or a pharmaceutical composition as described above or elsewhere in the present disclosure.

The present disclosure further provides a method of treating cancer in a patient by administering to the patient a therapeutically effective amount of an enzymatically methylated DNA or a pharmaceutical composition as described above or elsewhere in the present disclosure.

The present disclosure further provides a method of treating transplant rejection in a patient by administering to the patient a therapeutically effective amount of an enzymatically methylated DNA or a pharmaceutical composition as described above or elsewhere in the present disclosure.

The present disclosure further provides a plasmid pSV40-hBAX-BLa having the sequence of SEQ ID NO: 1.

The present disclosure further provides a plasmid mpSV40-hBAX-BLa having the sequence of SEQ ID NO: 1 and methylated on at least one CpG methylation site.

The present disclosure further provides a plasmid pSV40-sGAD55-BLa having the sequence of SEQ ID NO: 2.

The present disclosure further provides a plasmid mpSV40-sGAD55-BLa having the sequence of SEQ ID NO: 2 and methylated on at least one CpG methylation site.

The present disclosure further provides a plasmid pSV40-sGAD55+hBAX-BLa having the sequence of SEQ ID NO: 3.

The present disclosure further provides a plasmid m pSV40-sGAD55+hBAX-BLa having the sequenc of SEQ ID NO:3 and methylated on at least on CpG methylation site.

The present disclosure further provides a pharmaceutical composition including at least one of pSV40-hBAX-BLa, mpSV40-hBAX-BLa, pSV40-sGAD55-BLa, mpSV40-sGAD55-BLa, pSV40-sGAD55+hBAX-BLa, or m pSV40-sGAD55+hBAX-BLa; and a pharmaceutically acceptable carrier, excipient or diluent.

In more specific embodiments:
the composition includes both mpSV40-hBAX-BLa and mpSV40-sGAD55-BLa;
the mpSV40-hBAX-BLa and mpSV40-sGAD55-BLa are present in a ratio in a range from about 1:1 and 3:1;
the mpSV40-hBAX-BLa and mpSV40-sGAD55-BLa are present in a ratio in a range from about 1:4 and 3:1;
the mpSV40-hBAX-BLa and mpSV40-sGAD55-BLa are present in a ratio of 2:1;
the mpSV40-hBAX-BLa is methylated at a lower level than mpSV40-sGAD55-BLa;
the composition includes m pSV40-sGAD55+hBAX-BLa and at least one of mpSV40-hBAX-BLa or mpSV40-sGAD55-BLa;
the composition includes least one plasmid coupled to a delivery vehicle;
the composition includes a carbohydrate, an antioxidant, a chelating agent, a low molecular weight protein, another stabilizer or excipient, surfactant, or any combinations thereof;
the composition includes an additional therapeutic;
the pharmaceutical composition is suitable for injection;
the pharmaceutical composition is a solution or suspension of the plasmid;
the composition further includes water, saline solution, Ringer's solution, isotonic sodium chloride, a fixed oil, polyethylene glycol, glycerin, propylene glycol, glycerol, an injectable organic ester or other solvent or formulation carrier, an antibacterial agent, an antioxidant, a chelating agent, a buffer, and agents for the adjustment of tonicity, or any combinations thereof;
the pharmaceutical composition is suitable for topical administration;
the composition further includes a solution, emulsion, ointment, or gel base;
the pharmaceutical composition is suitable for transdermal administration;
the pharmaceutical composition includes a transdermal patch or iontophoresis device; and
the pharmaceutical composition is suitable for transmucosal administration.

The present disclosure further includes method of treating an autoimmune disease, an allergy, a transplant rejection, or cancer comprising administering to a subject with autoimmune disease, an allergy, a transplant rejection, or cancer a therapeutically effective amount of at least one of pSV40-hBAX-BLa, mpSV40-hBAX-BLa, pSV40-sGAD55-BLa, mpSV40-sGAD55-BLA, pSV40-sGAD55+hBAX-BLa, or m pSV40-sGAD55+hBAX-BLa as described above.

In more specific embodiments:
the subject has Type 1 Diabetes and the method includes administering a combination of mpSV40-hBAX-BLa and mpSV40-sGAD55-BLa;
the method includes administering mpSV40-hBAX-BLa and mpSV40-sGAD55-BLa in a ratio in a range from about 1:1 and 3:1;
the method includes administering mpSV40-hBAX-BLa and mpSV40-sGAD55-BLa in a ratio in a range from about 1:4 and 3:1;
the method includes administering mpSV40-hBAX-BLa and mpSV40-sGAD55-BLa in a ratio of 2:1 and
the subject has Type I Diabetes and the method includes administering at least m pSV40-sGAD55+hBAX-BLa.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic diagram of a CpG site only 2 bases in length.

FIG. 1B is a schematic diagram of a CpG site 20 bases in length, both beginning and ending in CG, and containing four total CG sequences.

FIG. 1C is a schematic diagram of an example plasmid with CpG sites.

FIG. 2A is a schematic diagram of the plasmid pSV40-sGAD55 with HpaII restriction sites indicated.

FIG. 2B is a schematic diagram of the plasmid pSV40-sGAD55 (also referred to as pSV40-sGAD55-BLa) with other restriction sites indicated.

FIG. 3 is a schematic diagram of the plasmid pSV40-hBAX-BLa with selected restriction sites indicated.

FIG. 4 is a diagram of an expected band pattern on an agarose gel after digestion of enzymatically methylated pSV40-sGAD55 with HpaII (1) or entirely unmethylated pSV40-sGAD55 with HpaII (2).

FIG. 5 is an agarose gel of HpaII-digested or MspI-digested DNA enzymatically methylated according to the present disclosure using 20 U M.SssI methyltransferase and of digested, unmethylated DNA.

FIG. 6 is a diagram showing methylation and analysis of linear (linearized from plasmid source DNA) DNA and plasmid (parental) source DNA.

FIG. 7A is an agarose gel of HpaII-digested DNA enzymatically methylated according to the present disclosure from linear or plasmid (parental) source DNA using various amounts of M.SssI methyltransferase.

FIG. 7B is the pyrosequencing results and corresponding agarose gel bands for plasmid (parental) source DNA and linear source DNA incubated with 1 U M.SssI methyltransferase.

FIG. 7C is the pyrosequencing results and corresponding agarose gel bands for plasmid (parental) source DNA and linear source DNA incubated with 2 U M.SssI methyltransferase.

FIG. 7D is the pyrosequencing results and corresponding agarose gel bands for plasmid (parental) source DNA and linear source DNA incubated with 3 U M.SssI methyltransferase.

FIG. 7E is the pyrosequencing results and corresponding agarose gel bands for plasmid (parental) source DNA and linear source DNA incubated with 4 U M.SssI methyltransferase.

FIG. 7F is a graph of methylation percentage at five CpG sites investigated by pyrosequencing in for plasmid (parental) source DNA and linear source DNA incubated with 1-4U of M.SssI methyltransferase.

FIG. 8A is an agarose gel of HpaII-digested DNA enzymatically methylated according to the present disclosure using various amounts of M.SssI methyltransferase.

FIG. 8B is another agarose gel of HpaII-digested DNA enzymatically methylated according to the present disclosure using various amounts of M.SssI methyltransferase.

FIG. 9 is an agarose gel of HpaII-digested DNA enzymatically methylated according to the present disclosure using M.SssI methyltransferase for incubation times as indicated.

FIG. 10 is an agarose gel of HpaII-digested DNA enzymatically methylated according to the present disclosure using M.SssI methyltransferase at the incubation temperatures indicated.

FIG. 11 is a series of agarose gels of HpaII-digested DNA enzymatically methylated according to the present disclosure using M.SssI methyltransferase at the incubation temperatures indicated.

FIG. 12 is an agarose gel of HpaII-digested DNA enzymatically methylated according to the present disclosure using M.SssI methyltransferase in the amounts indicated.

FIG. 13 is an agarose gel of HpaII-digested DNA either enzymatically methylated according to the present disclosure or bacterially methylated.

FIG. 14 is an agarose gel of HpaII-digested DNA either linear and ligated enzymatically methylated.

FIG. 15A is an agarose gel of HpaII-digested DNA from either plasmid and linear DNA samples enzymatically methylated with various concentration of Mg²⁺ in the reaction sample.

FIG. 15B is an agarose gel of HpaII-digested DNA from either plasmid and linear DNA samples enzymatically methylated with various concentration of Mg²⁺ in the reaction sample.

FIG. 15C is an agarose gel of and bacterially methylated samples not according to the present disclosure provided for comparative purposes (#5, #11, and #23).

FIG. 16 is an agarose gel of of HpaII-digested DNA enzymatically methylated according to the present disclosure using SAM in the amounts indicated.

FIG. 17 is a diagram of the plasmid pSV40-sGAD55+hBAX-BLa.

### DETAILED DESCRIPTION

The present disclosure relates to enzymatically methylated DNA and compositions containing enzymatically methylated DNA. More specifically, the DNA is methylated at sites containing a CpG sequence, i.e., 5′ C-phosphate-G-3′, where cytosine and guanine are separated by one phosphate group. The level of methylation at these sites allows targeted modulation of the immune response in a patient to whom the enzymatically methylated DNA or a composition containing the enzymatically methylated DNA is administered.

The present disclosure also provides methods of enzymatically methylating DNA at CpG sites. “Enzymatic methylation” is methylation of DNA using an extracellular methylation enzyme rather than methylation by enzymes located in a cell, such as a bacterial cell. Accordingly, enzymatic methylation occurs in vitro. DNA may be enzymatically methylated to target methylation levels or in target methylation patterns as disclosed herein that cause particular modulations of the immune response when the enzymatically methylated DNA is administered to a patient.

DNA methylation is also found at sites other than CpG sequences and accounts for approximately 0.02% of total methyl-cytosine in differentiated somatic cells in mammals, with a greater frequency in some brain tissues and embryonic stem cells. This type of methylation is referred to as non-CpG methylation and is encompassed by the present disclosure except in instances, such as in the “CpG site specific methylation level,” which are clearly directed to CpG methylation only. Non-CpG methylation may be catalyzed by DNMT3A and DNMT3B.

Enzymatic methylation methods offer any of a number of advantages as compared to bacterial or other cellular methylation methods. Of particular interest for pharmaceutical products containing enzymatically methylated DNA, enzymatic methylation does not require the use of cells containing antibiotic resistance genes associated with cellular methylation enzymes. This avoids the need to add selection antibiotics to the methylation samples, which antibiotics must then later be removed from the final pharmaceutical product before administration to patients. In some examples in which source DNA is artificially synthesized, the use of antibiotic resistance genes and associated selection antibiotic may be avoided throughout the entire production process of the pharmaceutical product, such that no antibiotic removal and no testing of the pharmaceutical product for residual antibiotics may be required. In other examples, in which the source DNA is synthesized using bacteria or another cellular vehicle, separation of the source DNA may remove most or all of any antibiotics present during formation of the source DNA, while no further antibiotics are required during methylation, resulting in some process efficiencies, testing of the pharmaceutical product for residual antibiotics may still be required.

Enzymatic methylation also reduces the need to remove cellular components from the enzymatically methylated DNA prior to use in a pharmaceutical product and, if source DNA is also artificially synthesized, may eliminate the need entirely. The methylation enzyme is likely still removed from the enzymatically methylated DNA prior to use in a pharmaceutical product, for example to avoid reactions, such as an immune response, to the enzyme, but removal of a simple protein, such as an enzyme, from DNA is a much simpler process than removing cellular components. In addition, testing to confirm enzyme removal is much simpler than testing to confirm removal of cellular components. Furthermore, in some embodiments it may not be necessary to remove the methylation enzyme, or it may be possible to simply reduce it to a low level. For example, in some embodiments, the enzymatically methylated DNA may simply be subjected to conditions that degrade the enzyme into non- or less-immunogenic fragments, while not harming the DNA. Many enzymatically methylated DNAs of the present disclosure need only be administered to a patient once, reducing concerns about immune reaction to residual methylating enzyme. Other enzymatically methylated DNAs, such as those used as cancer therapeutics, may seek to upregulate the immune response and may benefit from an adjuvant effect of the residual enzyme.

Enzymatic methylation may, therefore, simplify or decrease the cost of manufacturing methylated DNA as compared to cellular methods due to the absence or reduced need to use antibiotic resistance genes and antibiotics as well as cells in the process. In addition, enzymatic methylation may achieve these benefits by also speeding up the methylation process, using much simpler manufacturing equipment than is required for cell culture, eliminating many other expensive and time-consuming quality checks and quality-assurance processes, such as those designed to ensure that production cells have not been contaminated or experienced unacceptable genetic drift, and other efficiencies.

Furthermore, enzymatically methylated DNA may also exhibit greater batch-to-batch consistency in overall methylation levels and methylation patterns as compared to methods employing cells for methylation of DNA having a similar or identical sequence and/or structure.

The methylated DNA may contain a gene that encodes a pro-apoptotic protein and/or a determinant protein. A pro-apoptotic protein tends to cause apoptosis when expressed in a mammalian cell. A determinant protein is a trigger protein that causes a modulation in the immune response. Often the determinant protein is an allergen, an autoantigen, a cancer antigen, a donor antigen or sequestered tissue specific antigen, or a functional fragment of one or the foregoing. In some instances, the methylated DNA may also contain a gene that encodes a tolerance-inducing protein or a functional fragment thereof. Throughout the present specification, a reference to a pro-apoptotic protein, a determinant protein (whether generally or specifically as an allergen, autoantigen, cancer antigen, or donor antigen or sequestered tissue specific antigen), or a tolerance-inducing protein will include all functional fragments to such protein.

The present disclosure further provides methods of using enzymatically methylated DNA to modulate the immune response in a patient in a targeted fashion. The effects of enzymatically methylated DNA on the immune response may be determined in part by the proteins encoded by the enzymatically methylated DNA. The pro-apoptotic protein typically recruits dendritic cells (DC) to the area. The DCs then mediate later phases of the immune response, causing either a tolerogenic response if low levels of the determinant protein are present or a reactive immune response if high levels of the determinant protein are present. If a tolerance inducing protein is encoded by the enzymatically methylated DNA, it may also help induce a tolerogenic response. In addition, in some embodiments the enzymatically methylated DNA may only contain a gene for a pro-apoptotic protein, optionally with a tolerance inducing protein, but without any determinant protein gene. In such embodiments, levels of the determinant protein in the patient may cause either a tolerogenic or reactive immune response.

As used herein, the term “about” means±5% of the indicated range, value, or structure, unless otherwise indicated, the term “or” is inclusive and means “and/or,” and the terms “a” or “an” may refer to one or more than one.

Although the present specification may refer to embodiments or examples, elements disclosed in one embodiment or example may be combined with elements of other embodiments and examples unless such elements are clearly mutually exclusive or such combination would, based on the disclosure herein, be clearly not functional.

### Enzymatically Methylated DNA

Enzymatically methylated DNA may be methylated on at least one CpG site within the DNA. Enzymatic methylation may be confirmed by comparing the methylation level of the enzymatically methylated DNA to that of source DNA. If DNA has been enzymatically methylated, then after an enzymatic methylation process, the methylation level is higher than in the source DNA prior to the enzymatic methylation process. Enzymatic methylation may also be confirmed by a pattern of methylation not present in the source DNA.

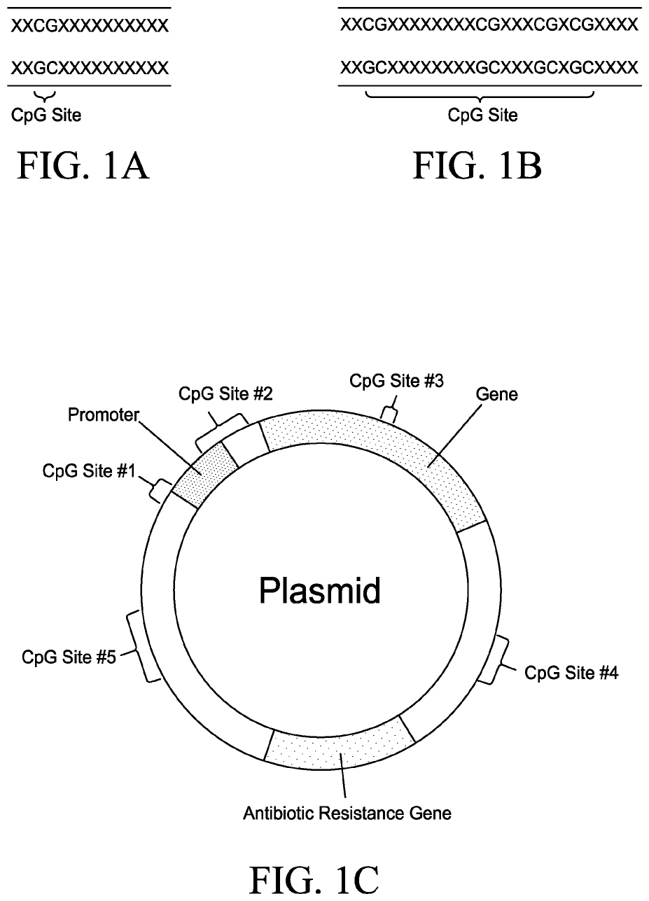

Methylation of DNA occurs at “CpG sites.” As used herein, a “CpG” site is a segment of DNA that has a sequence that includes at least one instance of “CG.” The segment of DNA may be in a range from 2 to 50 bases in length, 2 to 20 bases in length, or 2 to 10 bases in length, typically consisting of only CG in the case of a CpG site only 2 bases in length (FIG. 1A), or both beginning and ending in CG for longer sequences (FIG. 1B). A CpG site may have a sequence including more than one CG sequence (FIG. 1B).

Methylated DNA according to the present disclosure has at least one CpG site. Typically, the methylated DNA will have more than one CpG site. For example, the methylated DNA may have at least 2, 5, 10, 20, 50, or 100 CpG sites, or in a range from 2 to 500 CpG sites, 2 to 100 CpG sites, 10 to 500 CpG sites, or 10 to 100 CpG sites. An example plasmid methylated DNA with 5 CpG sites is provided in FIG. 1C. As the example shows, CpG sites may be located anywhere in the methylated DNA, including in the promoter or other regulatory DNA region, such as an enhancer portion, in a protein-encoding portion, or in a non-regulatory and non-coding portion of the methylated DNA.

As used herein, the “level of methylation” or “methylation level,” may refer to any of three types of methylation levels: “CpG site-specific methylation level,” “mean whole DNA methylation level,” or “fractional methylation level.” These terms are used in reference to an otherwise homogenous population of DNA molecules (e.g. with the same sequences and primarily with the same topography).

“CpG site specific methylation level” refers to the mean, across all DNA molecules in a sample, percentage of CG sequences within a given CpG site that are methylated. For example, if CpG Site #2 in FIG. 1C had the configuration shown in FIG. 1B, with four total CG sequences within the site, then to determine the “CpG site specific methylation level for CpG Site #2, one would look at the mean, across all DNA molecules in a sample, percentage of those CG sequences that were methylated. If, in most DNA molecules in the sample, only one CG sequence were methylated, then the CpG site specific methylation level for CpG Site #2 would be about 25%. If, in most DNA molecules in the sample, two CG sequences were methylated, then the CpG site specific methylation level for CpG Site #2 would be about 50%. If, in about half of the DNA molecules in the sample, only one CG sequence was methylated and, in about the other half of the DNA molecules in the sample, two CG sequences were methylated, then the CpG site specific methylation level for CpG Site #2 would be about 37.5%.

“Mean whole DNA methylation level,” refers to the average CpG site specific methylation level across all DNA molecules in the sample over all CpG sites. For example, if the plasmid of FIG. 1C had CpG site specific methylation levels for CpG Site #s 1, 2, 3, 4, and 5 of 15%, 85%, 72%, 24%, and 43%, respectively, then the mean whole DNA methylation level would be 47.8%.

“Fractional methylation level” refers to the mean, across all DNA molecules in a sample, percentage of CpG sites out of the total CpG sites contained in the DNA molecule that are methylated. The CpG site specific methylation level for each CpG site is not taken into account; if any CG sequence within a given CpG site is methylated, then the site is considered methylated. Using the plasmid of FIG. 1C as an example, if, on average, all DNA molecules in a sample were methylated at CpG Site #s 1, 2, and 4, then the fractional methylation level for the plasmid would be about 60%. In another example using the plasmid of FIG. 1C, if, on average, about half of all DNA molecules in the sample were methylated at CpG Site #s 1, 2, and 4 and, on average, about half of all DNA molecules in the sample were methylated at CpG Site #s 2, 3, and 5, the fractional methylation level for the plasmid would still be approximately 60%.

In enzymatically methylated DNA where the methylation enzyme also methylates cytosine at other sites, a “C site specific methylation level” may be calculated for any methylation site containing a cytosine. A “mean whole DNA methylation level for all C sites” may be calculated using the “C site specific methylation levels” and a “fractional methylation level for all C sites” may be calculated for all C sites on the DNA molecule. Methylation levels for these measures that include all C sites may be similar to those disclosed herein for CpG sites. In addition, methylation patterns may be similar, although additional gel analysis may be possible using restriction enzymes that are sensitive to C methylation at non-CpG sites.

The methylation level of DNA can be determined using known methods, such as a bead array, PCR and sequencing, bisulfite conversion and pyrosequencing, methylation-specific PCR, PCR with high resolution melting, or COLD-PCR to detect unmethylated islands or gel-based methods as provided herein. These techniques are described in more detail in Kurdyukov and Bullock, “DNA Methylation Analysis: Choosing the Right Method,” Biology (Basel) 5 (1): 3, (2016), Sections 4 and Table 2 of which, and further publications (listed below) referenced in Section 4 of which are incorporated by reference herein with respect to their disclosures of methylation detection techniques. These techniques may be used to calculate different aspects of methylation level. For example, bisulfite conversion and pyrosequencing typically provides CpG site specific methylation levels for specific CpG sites. If pyrosequencing is conducted for each CpG site, then it may be used to calculate mean whole DNA methylation level.

Methylation level can also be determined by incubating a representative portion of a DNA sample with a CpG methylation-sensitive restriction enzyme as disclosed herein and then conducting agarose gel electrophoresis with the sample. The presence or absences of bands of certain sizes, based on the number of bases between potential restriction sites and/or the size of uncleaved methylated DNA, as well as the relative intensities of these bands may also provide quantitative or quality-control level qualitative information regarding all three types of methylation levels.

Each of the three types of methylation levels of enzymatically methylated DNA and the methylation level at any specific CpG site may be controlled to range from 0% to 100% depending on the target immune modulation effect. Typically, lower levels of methylation are associated with bacteria lacking methyltransferase and are more likely to cause an immune response. For example, in bacteria lacking a methyltransferase gene, the mean whole DNA methylation level is about 3%. However, higher levels of methylation, particularly high levels of CpG site specific methylation of CpG sites in a promoter, may modulate with gene expression.

The CpG site specific methylation level for at least one CpG site, and up to 50%, 75%, or 90% of CpG sites in an enzymatically methylated DNA and/or the mean whole DNA methylation level for an enzymatically methylated DNA may, therefore, typically be at least 5%, at least 10%, at least 15%, or at least 30% to lessen the immune response to the DNA. In some examples the CpG site specific methylation level for at least one CpG site and/or the whole DNA methylation level may be in a range from greater than 5% to 100%, greater than 5% to 70%, greater than 5% to 60%, greater than 5% to 50%, 5 greater than % to 30%, greater than 5% to 20%, greater than 5% to 25%, greater than 5% to 15%, greater than 5% to 10%, greater than 10% to 100%, greater than 10% to 70%, greater than 10% to 60%, greater than 10% to 50%, greater than 10% to 30%, greater than 10% to 25%, greater than 10% to 20%, greater than 10% to 15%, greater than 30% to 100%, greater than 30% to 70%, greater than 30% and 60%, or greater than 30% to 50%.

In some examples the fractional methylation level may be at least 5%, at least 10%, at least 15%, or at least 30%, or in a range from greater than 1% to 100%, greater than 5% to 100%, greater than 10% to 100%, greater than 15% to 100%, greater than 20% to 100%, greater than 25% to 100%, greater than 30% to 100%, greater than 35% to 100%, greater than 45% to 100%, greater than 50% to 100%, greater than 55% to 100%, greater than 60% to 100%, greater than 65% to 100%, greater than 70% to 100%, greater than 75% to 100%, greater than 80% to 100%, greater than 85% to 100%, greater than 90% to 100%, greater than 95% to 100%, greater than 1% to 95%, greater than 5% to 95%, greater than 10% to 95%, greater than 15% to 95%, greater than 20% to 95%, greater than 25% to 95%, greater than 30% to 95%, greater than 40% to 95%, greater than 45% to 95%, greater than 50% to 95%, greater than 55% to 95%, greater than 60% to 95%, greater than 65% to 95%, greater than 70% to 95%, greater than 75% to 95%, greater than 80% to 95%, greater than 85% to 95%, greater than 90% to 95%, greater than 1% to 70%, greater than 5% to 70%, greater than 10% to 70%, greater than 15% to 70%, greater than 20% to 70%, greater than 25% to 70%, greater than 30% to 70%, greater than 35% to 70%, greater than 40% to 70%, greater than 45% to 70%, greater than 50% to 70%, greater than 55% to 75%, greater than 60% to 70%, greater than 65% to 70%, greater than 1% to 60%, greater than 5% to 60%, greater than 10% to 60%, greater than 15% to 60%, greater than 20% to 60%, greater than 25% to 60%, greater than 30% to 60%, greater than 35% to 60%, greater than 40% to 60%, greater than 45% to 60%, greater than 50% to 60%, greater than 55% to 60%, greater than 1% to 30%, greater than 5% to 30%, greater than 10% to 30%, greater than 15% to 30%, greater than 20% to 30%, greater than 25% to 30%, greater than 1% to 15%, greater than 5% to 15%, or greater than 10% to 15%.

When M.Sssi methyltransferase is used as the enzyme and reaction time is one hour, a range from 1 U to 3 U may be used to achieve a mean whole DNA methylation level in a range of greater than 30% to 60%.

For any type of methylation level, the methylated DNA may have a stimulating methylation level or an attenuating methylation level. DNA with a stimulating methylation level may have a methylation level of greater than zero, but 30% or less, such as in a range from greater than 5% to 30% from greater than 5% to 15%, or greater than 15% to 30%. DNA with an attenuating methylation level may have a methylation level of greater than 30%, such as in a range from greater than 30% to 100% or greater than 30% to 60%. Enzymatically methylated DNA may be prepared using source DNA, which is typically

DNA with a lower methylation level of CpG sites than the enzymatically methylated DNA. Depending on how source DNA was produced, it may have no methylation of CpG sites at all, or very low levels of CpG methylation. Source DNA may also have a different methylation pattern than enzymatically methylated DNA or a lower methylation level at a specific CpG site. Source DNA may be produced in bacteria that lack methyltransferase genes, or in bacteria that have methyltransferase genes. The level of methylation of the source DNA may reflect whether the bacteria contain a methyltransferase gene.

Enzymatically methylated DNA may be in any of a variety of structures. For example, enzymatically methylated DNA may be a linear polynucleotide, a plasmid, an artificial chromosome, or a covalently closed linear double-stranded DNA structure, such as a structure with non-coding loops at both ends that, along with base pairing, control the overall shape of the DNA. Some enzymatically methylated DNAs may include a structural DNA element, a nucleotide element, or an associated protein which causes the DNA to adopt a particular structure. In general, enzymatically methylated DNA may have any higher-level three-dimensional structures, such as a supercoiling or nucleosome structures resulting from association with histones or other nucleosome or chromatin-forming proteins. It may also have any strand-level three-dimensional structure, such as hairpins including hairpin loops (stem loops) or imperfect hairpin loops, pseudoknots, or any one of the various types of double helix structures, such as A-DNA, B-DNA, or Z-DNA double helix structures.

Enzymatically methylated DNA of the present disclosure may be of any size, for example ranging from a short expression cassette to an artificial chromosome. Typically, enzymatically methylated DNA will be isolated.

In addition to having certain levels of methylation, enzymatically methylated DNA may also have a certain methylation pattern, as may be determined by digestion with at least one CpG methylation-sensitive restriction enzyme, such as HpaII, which only cleaves DNA at CCGG sites in the absence of methylation at those sites. Other CpG methylation-sensitive restriction enzymes whose activity is blocked by CpG methylation include: BfoI, AatII, AjiI, Bsh1236I (BstUI), Bsh1285I (BsiEI), BshTI (AgeI), Bsp119I (BstBI), Bsp681 (NruI), Bsu15I (ClaI), Cfr10I (BsrFI), Cfr42I (SacII), CpolI (RsrII), CseI (HgalI), Eco105I (SnaBI), Eco47III (AfeI), Eco52I (EagI), Eco72I (PmII), EheI (SfoI), Esp3I (BsmBI), FspAI, HhaI, Hin1I (BsaHI), Hin6I (HinP1I), Kpn2I (BspEI), MauBI, MluI, MreI (Sse232I), NotI, NsblI (FspI), PauI (BssHII), PdiI (NaeI), Pf123II (BsiWI), Ppu21I (BsaAI), Psp140gI (AclI), PvuI, SalI, SfaAI (AsiSI), SgrDI, Sgsl (Ascl), SmaI, SsiI (AciI), SspDI (KasI), Tail (MaeII), and TauI. In addition, CpG methylation-sensitive restriction enzymes whose activity is impaired by CpG methylation include: BcnI (NciI), Cfr9I (Xmal), Eco88I (Aval), MbiI (BsrBI), and XhoI.

“Enzymatic methylation pattern” refers to which of the available CpG sites of an enzymatically methylated DNA are methylated. A particular enzymatic methylation pattern may be associated with therapeutic efficacy of the enzymatically methylated DNA.

Enzymatically methylated DNA may have a methylation pattern that is the same as that achieved by a host cell containing the same methylation enzyme used to produce the enzymatically methylated DNA. Alternatively, enzymatically methylated DNA may have a methylation pattern different from that obtained using the same methylation enzyme in a host cell or even not achievable using cellular methylation, particularly bacterial or yeast methylation.

In addition, enzymatically methylated DNA may have CpG site specific methylation levels that are not achievable using cellular methylation, particularly bacterial or yeast methylation. In addition, CpG site-specific methylation is typically not achievable when using bacterial methylation or other cellular methylation, whereas in enzymatic methylation certain CpG sites may be targeted for methylation, while others are not and remain unmethylated or sparsely methylated.

Enzymatically methylated DNA of the present disclosure may be present in a sample that is highly homogenous in terms of any one, two or all three types of methylation levels (expressed as the percent of individual DNA molecules having a methylation level within 5% of the mean methylation level for the DNA sample) and/or methylation pattern (expressed as the percent of individual DNA molecules having a specific methylation pattern). For example, the enzymatically methylated DNA may be at least about 70% homogenous, about 75% homogenous, about 80% homogenous, about 85% homogenous, about 90% homogenous, about 95% homogenous, or about 99% homogenous or have a homogeneity in a range from any two values of about 70%, about 80%, about 90%, about 85%, about 90%, about 95%, about 99%, or about 99.9% with respect to any methylation level type or methylation pattern.

In addition, the batch-to-batch variation of any type of methylation level or methylation pattern between separately prepared enzymatically methylated DNA samples prepared using the same source DNA under the same methylation process conditions may be less than about 50%, less than about 40%, less than about 30%, less than about 20%, less than about 10%, or less than about 5%, or in a range from about 0% to less than about 50%, about 0% to less than about 40%, about 0% to less than about 30%, about 0% to less than about 20%, about 0% to less than about 10%, or about 0% to less than about 5%. In general, batch-to-batch variation may be due to imprecise temperature control, batch size, and other minor variations in process parameters that can be readily identified and corrected if batch-to-batch variation is too high. In contrast, batch-to-batch variations, particularly in methylation levels, tend to be high in cellular methylation methods and identifying and correcting causes of such variation is difficult.

Enzymatically methylated DNA of the present disclosure encodes at least one protein. In some embodiments, this protein may be a pro-apoptotic protein, a determinant protein, or a tolerance-inducing protein. In some embodiments, the enzymatically methylated DNA may include an expression cassette for expression of the encoded protein. Such an expression cassette includes at least a gene having a sequence that encodes at least one protein and may also contain at least one regulatory element, such as a promoter.

A pro-apoptotic protein tends to cause apoptosis when expressed in a mammalian cell. A pro-apoptotic protein may activate apoptosis mechanisms directly or indirectly. A functional fragment of a pro-apoptotic protein may also tend to cause apoptosis when expressed in a mammalian cell. Suitable exemplary pro-apoptotic proteins include BCL2 associated X protein (Bax), BCL2-antagonist/killer 1 (Bak), BCL-2-interacting mediator of cell death (Bim), p53 up-regulated modulator of apoptosis (Puma), BCL-2 associated agonist of cell death (Bad), BCL-2-interacting killer (Bik), phorbol-12-myristate-13-acetate-induced protein 1 (Noxa), BCL-2 modifying factor (Bmf), hara-kiri (Hrk), BH3 interacting-domain death agonist (Bid), FAS, a FAS receptor, second mitochondria-derived activator of caspase (Smac), HtrA serine peptidase 2 (Omi/HtrA2), Septin 4 (ARTS/Sep4), Death Receptor 4 (DR4), Death Receptor 5 (DRS), apoptosis inducing factor (AIF), cytochrome C, endonuclease G, Caspase-activated deoxyribonuclease (CAD), apoptosis protease activating factor-1 (APAF-1) a Tumor Necrosis Factor Receptor, an apoptosis-inducing caspase mutant, an apoptosis-inducing modified caspase, an apoptosis-inducing survivin mutant, an apoptosis-inducing modified survivin, an apoptosis-inducing IAP mutant, and IAP antagonist. A suitable exemplary pro-apoptotic protein functional fragment includes the BH3 domain of Bax.

A tolerance-inducing protein helps modulate the immune response to be tolerogenic. For example, the tolerance-inducing protein may be human complementarity determining region 1 (hCDRI). A determinant protein causes a modulation in the immune response to the determinant protein in the patient. For example, the determinant protein may be the target of a harmful immune response, such as an allergen, autoantigen, or donor antigen or sequestered tissue specific antigen, or a target that is not recognized by the immune system, but beneficially should be, such as a cancer antigen. A functional fragment of a determinant protein is a fragment that is also able to induce modulation of the immune response, and may often be an antigenic fragment. In most embodiments, the modulation of the immune response induced by the functional fragment will be the same type of modulation of the immune response, e.g. upregulation, downregulation, induction of a tolerogenic immune response, or induction of a reactive immune response, as is sought with respect to the full-length protein. For example, a functional fragment of an allergen may also induce a tolerogenic response to the full-length allergen protein. In specific examples, the functional fragment may be a fragment recognized by the immune system, such as an epitope recognized by an antibody or a T cell receptor.

Often the determinant protein may be an allergen, an autoantigen, a cancer antigen, or a donor antigen or sequestered tissue specific antigen. The determinant protein may also be modified to alter its immune modulatory effect. For example, the determinant protein may be modified to include a peptide segment that targets the determinant protein to the cellular secretory pathway, resulting in secreted determinant protein, which is more likely to interact with the immune system to prompt a Th2 type immune response. In contrast, membrane-bound proteins are more likely to prompt a Th1 type immune response. Similarly, a tolerance-inducing protein may also be modified to include a peptide segment that targets the tolerance-inducing protein to the cellular secretory pathway, resulting in secreted tolerance-inducing protein, and thereby improving the tolerogenic effects of such protein.

As used herein, an “allergen” is a protein that causes an IgE-mediated immune response in a patient, but that is not otherwise harmful to the patient or found in an organism, such as a parasite, that is harmful to the patient. Common allergens include pollen proteins, such as grass and tree pollen, animal dander proteins, such as dog and cat dander proteins, dust mite proteins, insect proteins, such as those injected into the body through an insect bit or sting, protein-based medications, such as some therapeutic antibodies or proteins, mold proteins, and food proteins, such as nut, fruit, shellfish, egg, and milk, particularly cow's milk, proteins.

As used herein, an “autoantigen” is an endogenous protein that stimulates the production of autoantibodies, as in an autoimmune reaction, to the protein. For example, in the context of this disclosure carbonic anhydrase II, chromogranin, collagen, CYP2D6 (cytochrome P450, family 2, subfamily Device 400, polypeptide 6), glutamic acid decarboxylase (GAD), secreted glutamic acid decarboxylase 55 (sGAD), islet cell antigen 512 (IA2), islet-specific glucose-6-phosphatase catalytic subunit-related protein (IGRP), insulin, myelin basic protein, human Ninein (hNinein), Ro 60 kDa, SRY-box containing gene 10 (SOX-10), zinc transporter 8 (ZnT8), thyroglobulin, thyroperoxidase, thyroid stimulating hormone receptor, chromogranin A (ChgA), islet amyloid polypeptide (IAPP), peripherin, tetraspanin-7, prolyl-4-hydroxylase β (P4Hb), glucose-regulated protein 78 (GRP78), urocortin-3, insulin gene enhancer protein isl-1, 21OH hydroxylase, 17OH hydroxylase, H+/K+ ATPase, transglutaminase, tyrosinase, tyrosinase-related protein-2, myelin basic protein, proteolipid protein, desmogleins, hepatocyte antigens, cytochrome; P450-1A2, acetylcholine receptor, 2-oxoacid dehydrogenase complexes, trichohyalin, cathelicidin LL-37, melanocytic ADAMTSL5, lipid antigen PLA2G4D and keratin 17 are autoantigens.

As used herein, a “cancer antigen” is a protein associated with a cancer cell, often a mutated protein. In some embodiments, the cancer antigen may be an antigen that is recognized or expected to be recognized by the immune system in response to checkpoint inhibitor therapy. Cancer antigens may be a Burkitt lymphoma, neuroblastoma, melanoma, osteosarcoma, renal cell carcinoma, breast cancer, prostate cancer, lung carcinoma, colon cancer, germ cell tumors, ovarian cancer, or hepatocellular carcinoma cancer antigen. Specific cancer antigens may comprise alphafetoprotein (AFP), carcinoembryonic antigen (CEA), cancer antigen-125 (CA-125), mucin 1, cell surface associated (MUC1), epithelial tumor antigen (ETA), tyrisonase, melanoma-associated antigen (MAGE), and mutants of ras and p53 and include, for example, WT1, mesothelin, KRAS, ROR1, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD19, CD20, CD22, CD56, CD23, CD24, CD30, CD33, CD37, CD44v7/8, CD38, CD56, CD123, CA125, c-MET, FcRH5, folate receptor α, VEGF-α, VEGFR1, VEGFR2, IL-13Rα2, IL-11Rα, MAGE-A1, PSA, ephrin A2, ephrin B2, NKG2D, NY-ESO-1, TAG-72, NY-ESO, 5T4, BCMA, FAP, Carbonic anhydrase 9, BRAF, α-fetoprotein, MAGE-A3, MAGE-A4, SSX-2, PRAME, HA-1, B2M, ETA, tyrosinase, NRAS, or CEA antigen.

As used herein, a “donor antigen” is a protein from an allograft that was transplanted into a patient, typically to take the place of defective or absent cells or tissues. The patient's immune system has recognized or may potentially recognize the donor antigen as foreign and, as a result, produces leukocytes or antibodies that target the donor antigen and the allograft containing it. Allografts that may contain donor antigens include islet cells, hearts, lungs, kidneys and livers.

A “sequestered tissue specific antigen” is an antigen located in a transplanted organ or tissue that is not normally available for immune recognition, but becomes available due to the transplantation process.

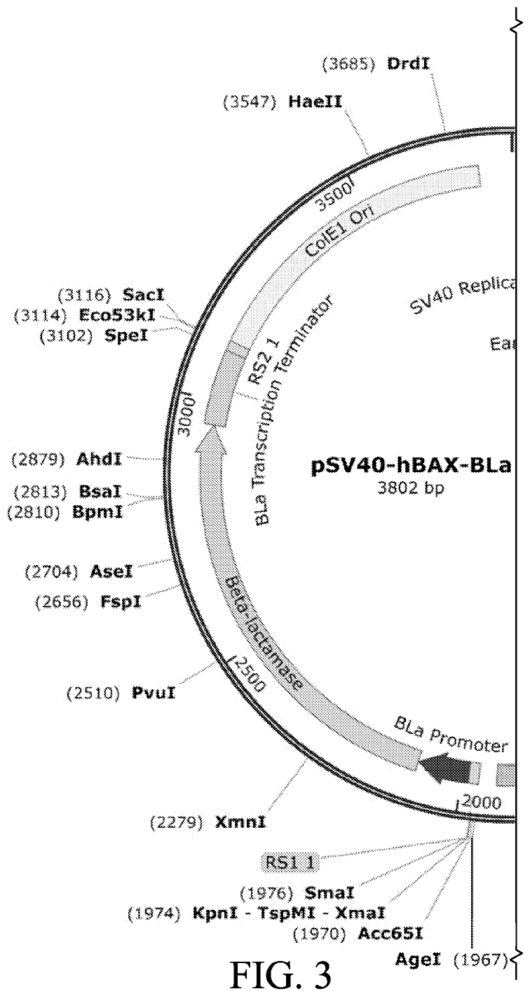

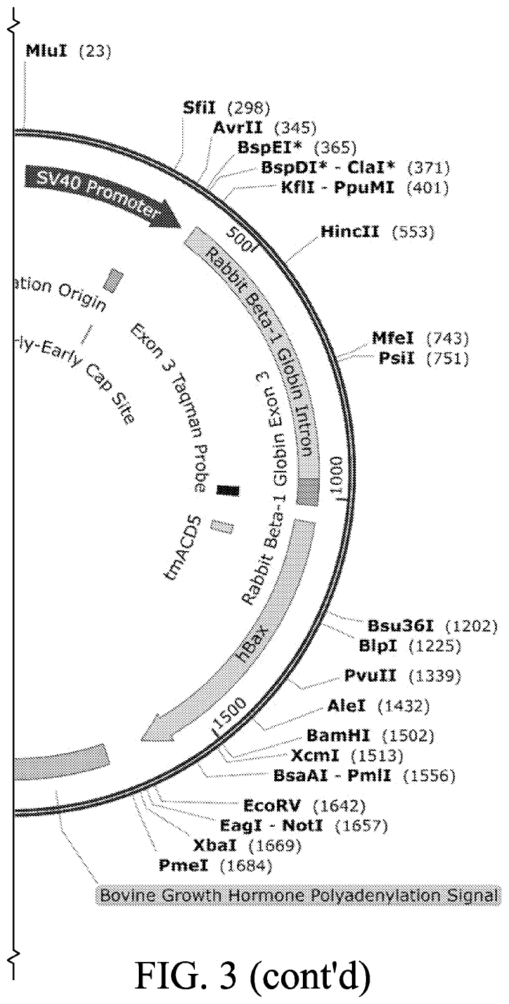

According to specific embodiments, the present disclosure provides an enzymatically methylated DNA with a stimulating methylation level including a gene encoding a pro-apoptotic protein, a tolerance-inducing protein, or a cancer antigen. In a more particular embodiment, the enzymatically methylated DNA may have a stimulating methylation level of at least one and up to all CpG sites within the promoter controlling expression of the pro-apoptotic protein, tolerance-inducing protein, or cancer antigen. This allows for high levels of protein expression to facilitate, in the case of the pro-apoptotic protein, apoptosis, in the case of the tolerance-inducing protein, a tolerogenic immune response, and in the case of the cancer antigen, a reactive immune response. In particular, the present disclosure provides an enzymatically methylated expression cassette with a stimulating methylation level containing BAX. The expression cassette may particularly be contained in a plasmid or synthetic DNA. In one specific embodiment, the enzymatically methylated DNA is enzymatically methylated pSV40-hBAX-BLa (FIG. 3) with a stimulating methylation level. The sequence of pSV40-hBAX-Bla is provided in Table 1. Plasmid pSV40-hBAX-BLa expresses the protein BCL2 associated X protein (BAX). BAX is a pro-apoptotic protein. BAX may also recruit dendritic cells to the area, such as the cell or tissue, where it is expressed. Plasmid pSV40-hBAX-BLa also contains the SV40 promoter, which controls expression of bax. Plasmid pSV40-hBAX-BLa further includes the beta-lactamase gene under control of the BLa promoter to allow beta lactam selection of bacteria, particularly E. coli containing the plasmid, particularly during production of the plasmid. Plasmid pSV40-hBAX-BLa is optimized for replication in E. coli. A variant of plasmid pSV40-hBAX-BLa that lacks elements specific for production in E. coli, such as the beta-lactamase gene under control of the BLA promoter or sequences or other elements optimized for replication in E. coli, may be used in connection enzymatic methylation, such as that of the present disclosure, if coupled with non-bacterial replication of the plasmid.

In a more specific embodiment, the present disclosure provides an enzymatically methylated expression cassette with a stimulating methylation level containing both a gene encoding a pro-apoptotic protein, in particular BAX, and a gene encoding a cancer antigen.

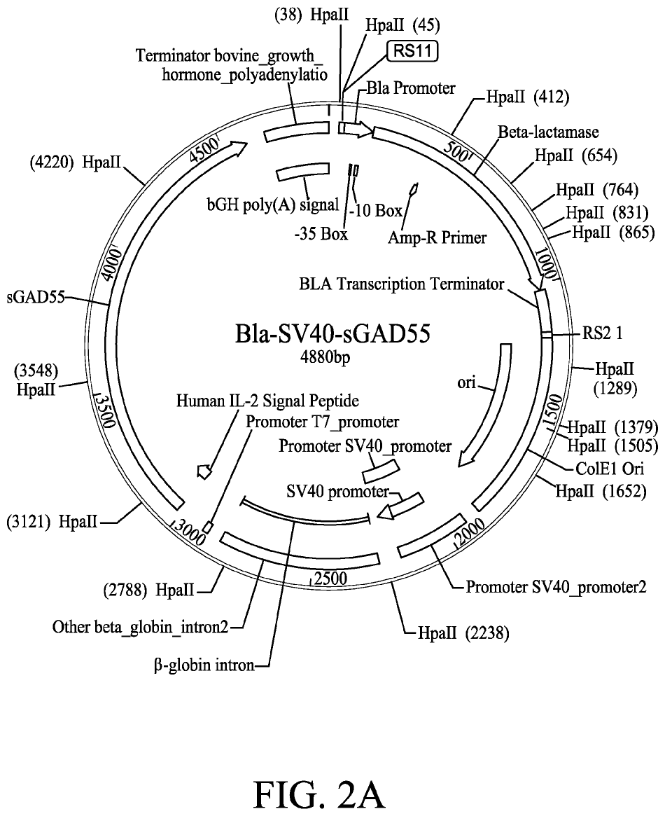

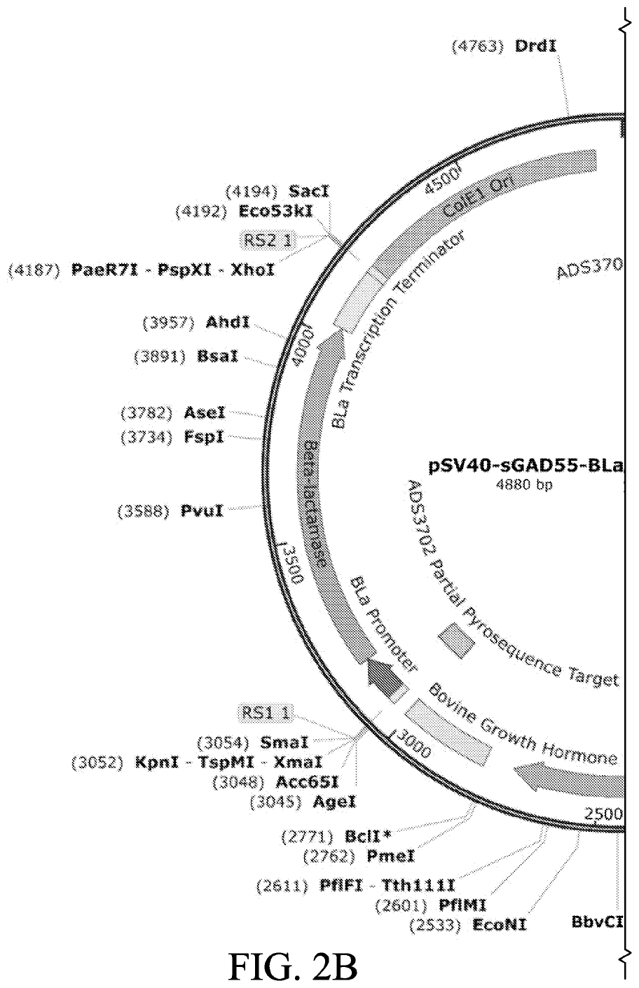

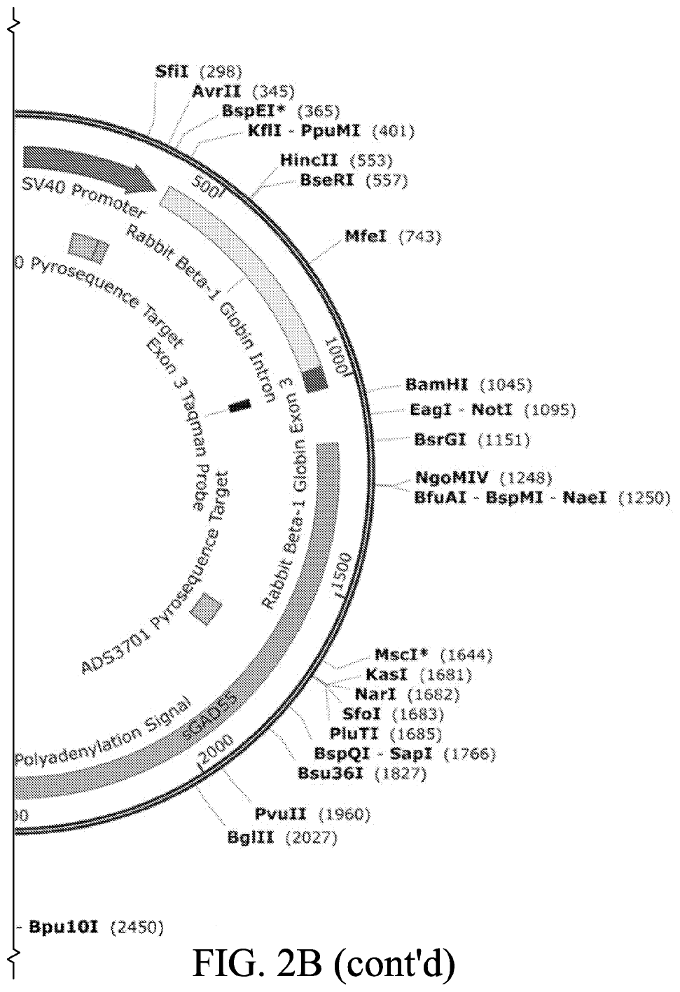

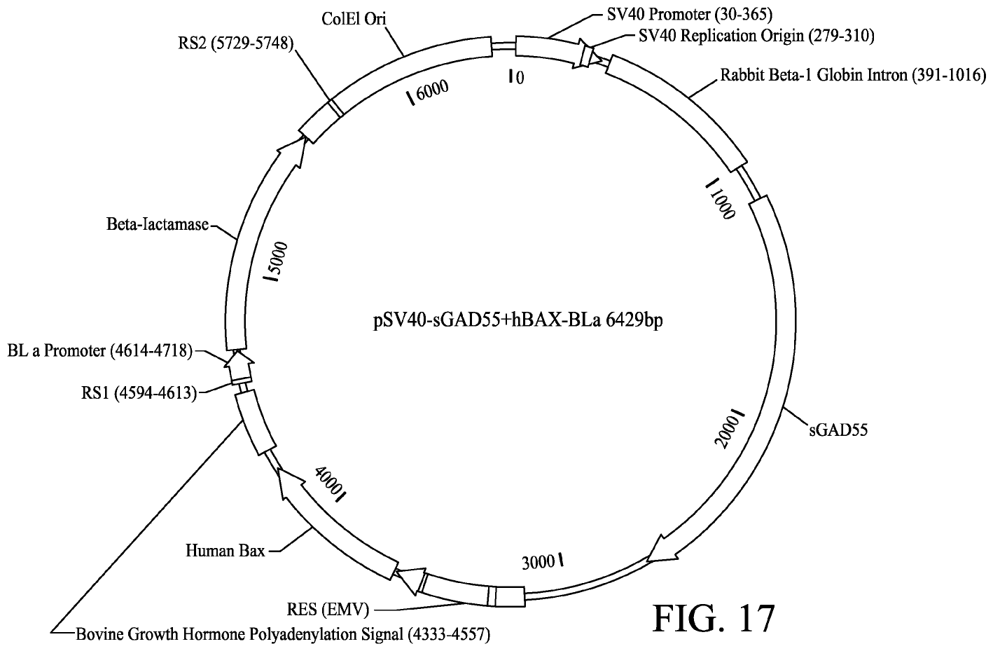

According to specific embodiments, the present disclosure provides an enzymatically methylated DNA with an attenuating methylation level including a gene encoding a pro-apoptotic protein, an allergen, an autoantigen, or a donor antigen, or sequestered tissue specific antigen. In a more particular embodiment, the enzymatically methylated DNA may have an attenuating methylation level of at least one and up to all CpG sites within the promoter controlling expression of the pro-apoptotic protein, allergen, autoantigen, or donor antigen or sequestered tissue specific antigen. This allows for some expression of the protein, but keeps levels low enough to cause a tolerogenic immune response to the allergen, autoantigen, or donor antigen or sequestered tissue specific antigen. In the case of the pro-apoptotic protein, it allows some expression and induction of apoptosis, while otherwise limiting any immune response to poorly methylated DNA. In one specific embodiment, the enzymatically methylated DNA is enzymatically methylated pSV40-sGAD55 (FIG. 2A and FIG. 2B) or enzymativcally methylated pSV40-sGAD55+hBAX-BLa (FIG. 17) with an attenuating methylation level. The sequence of pSV40-sGAD55 is provided in Table 1. Plasmid pSV40-sGAD55-BLa expresses a secreted form of the protein glutamic acid decarboxylase (sGAD). GAD is a common autoantigen in Type I Diabetes. Expression of sGAD, particularly at low levels, in the presence of BAX and/or dendritic cells, may induce a tolerogenic immune response to GAD. This tolerogenic immune response may be beneficial to subjects at risk of developing Type I Diabetes or subjects with Type I Diabetes by preventing the development of the disease, limiting the progression of the disease, or decreasing at least one symptom of the disease. In particular, the tolerogenic immune response may decrease death if islet cells in the subject's pancreas, increase the amount of insulin produced by the subject, reduce the deviation of the subject's blood sugar levels from normal levels in either a fasting or fed state, and/or reduce the need for insulin therapeutics in the subject, for example by reducing the dose of insulin therapeutics or the frequency of administration of insulin therapeutics required to control blood sugar levels in the subject. Plasmid pSV40-sGAD55-BLa also contains the SV40 promoter, which controls expression of sGAD. Plasmid pSV40-sGAD55-BLa further includes the beta-lactamase gene under control of the BLa promoter to allow beta lactam selection of bacteria, particularly E. coli containing the plasmid, particularly during production of the plasmid. Plasmid pSV40-sGAD55-BLa is optimized for replication in E. coli. A variant of plasmid pSV40-sGAD55-BLa that lacks elements specific for production in E. coli, such as the beta-lactamase gene under control of the BLA promoter or sequences or other elements optimized for replication in E. coli, may be used in connection enzymatic methylation, such as that of the present disclosure, if coupled with non-bacterial replication of the plasmid.

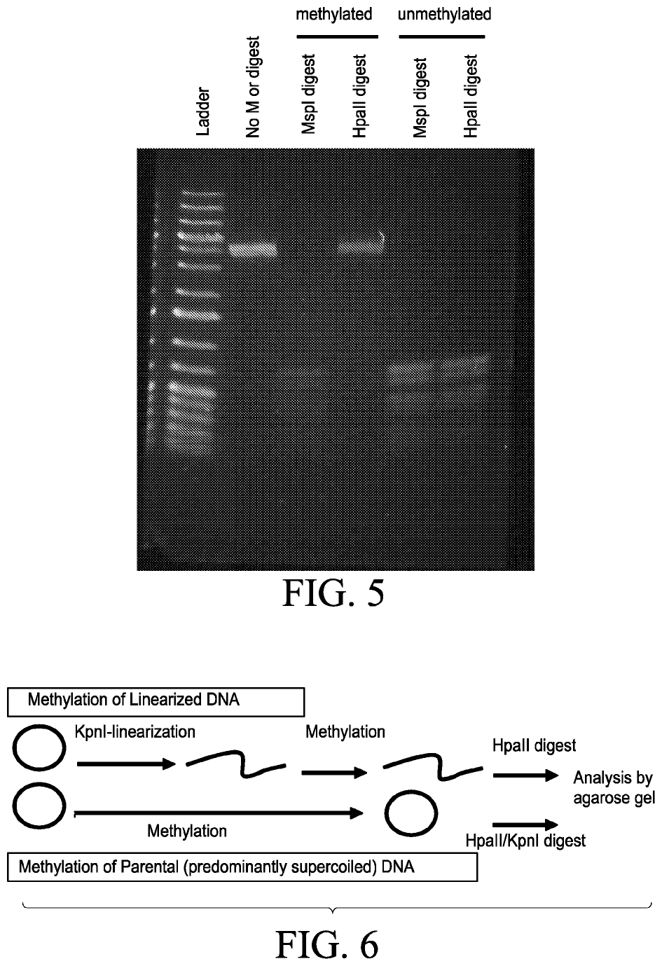

Plasmid pSV40-sGAD55+hBAX-BLa is illustrated in FIG. 5 and has a sequence as set forth in Table 1.

Plasmid pSV40-sGAD55+hBAX-BLa expresses sGAD and BAX. Plasmid pSV40-sGAD55+hBAX-BLa contains the SV40 promoter, which controls expression of both proteins. pSV40-sGAD55+hBAX-BLa also contains an IRES (EMV) sequence between the sequence encoding sGAD and that encoding BAX to allow a mammalian cell to initiate translation of BAX in a cap-independent manner and to avoid the need for careful coordination of sGAD and BAX reading frames. pSV40-sGAD55+hBAX-BLa further includes the beta-lactamase gene under control of the BLa promoter to allow beta lactam selection of bacteria, particularly E. coli containing the plasmid, particularly during production of the plasmid. Plasmid pSV40-sGAD55+hBAX-BLa is optimized for replication in E. coli. A variant of plasmid pSV40-sGAD55+hBAX-BLa that lacks elements specific for production in E. coli, such as the beta-lactamase gene under control of the BLA promoter or sequences or other elements optimized for replication in E. coli, may be used in connection enzymatic methylation, such as that of the present disclosure, if coupled with non-bacterial replication of the plasmid.

In another particular, the present disclosure provides an enzymatically methylated expression cassette with an attenuating methylation level containing the gene encoding the pro-apoptotic protein, particularly BAX, allergen, autoantigen, donor antigen or sequestered tissue specific antigen, a tolerance-inducing protein. The expression cassette may particularly be contained in a plasmid or synthetically produced DNA.

In one specific embodiment, the present disclosure provides an enzymatically methylated expression cassette with an attenuating methylation level containing both a gene encoding a pro-apoptotic protein, in particular BAX, and a gene encoding an allergen, an autoantigen, or a donor antigen or sequestered tissue specific antigen.

### Enzymatic Methylation Methods

The present disclosure also provides methods of enzymatically methylating source DNA to produce enzymatically methylated DNA, which may include any enzymatically methylated DNA described herein.

Source DNA may be any DNA containing at least one CpG site. Source DNA may have any methylation level. The methylation levels and methylation pattern of the source DNA will vary depending on its production method. In artificially synthesized source DNA, all methylation levels may be about 0%. However, source DNA produced in a host cell, such as a bacterial, yeast, other fungal, insect, or mammalian cell will have methylation levels dictated by the host cell. For example, even source DNA produced in bacterial lacking a methyltransferase gene will typically have a mean whole DNA methylation level of 3% or less. Source DNA may be demethylated prior to enzymatic methylation by, for example, cationic concentration. In specific embodiments, source DNA may be produced via artificial synthesis or in bacteria lacking a methyltransferase gene in order to limit methylation of the source DNA prior to enzymatic methylation.

Source DNA that is produced by artificial synthesis may also allow antibiotic resistance genes to be omitted from the source DNA and ultimately the enzymatically methylated DNA, which makes the enzymatically methylated DNA shorter and/or less complex and eliminates the need to add to the cell culture antibiotics when producing the source DNA that must later be removed prior to administering the enzymatically methylated DNA to a patient.

Source DNA may also have a more stable structure after formation if it is produced by artificial synthesis, which may allow more consistent results of enzymatic methylation as well as improved selection of particular methylation levels or methylation patterns, and more specific control over whether specific CpG sites are methylated and to what level as compared to cellularly-produced source DNA.

Methylation levels and methylation patterns may also be affected by the total number and location of CpG sites in the source DNA and the number of CG sequences the CpG sites contain. Accordingly, the source DNA may have features to increase or decrease the total number of CpG sites or GC sequences within CpG sites, or to place CpG sites in particular locations. In one exemplary embodiment, a codon within the portion of the source DNA encoding the pro-apoptotic protein or the determinant protein may be replaced with a degenerate codon that results in either the creation or elimination of a CpG site within the source DNA. In another exemplary embodiment, at least one CpG site, such as, for example a string of 5-50, 10-50, or 20-50 CG sequences may be introduced into the source DNA, particularly in a non-coding region, to increase the overall methylation level of the enzymatically methylated DNA. In still another embodiment, it is contemplated that a CpG site in a promoter may be modified to remove or add at least one CG sequence, thereby affecting whether the promoter may be methylated, where it is methylated, and the total number of methyl groups it may contain.

Source DNA may have any structure. In some examples the source DNA simply has the same structure as the enzymatically methylated DNA produced from it. In embodiments where the source DNA is artificially synthesized and methylated in the same reaction sample, it is particularly likely that the source DNA and enzymatically methylated DNA will have the same structure.

Source DNA may also have a structure that is different from the enzymatically methylated DNA for administration to a patient, particularly as contained in a pharmaceutical composition.

In some embodiments, source DNA may be a plasmid that is linearized prior to methylation, or source DNA may be linear and ligated after methylation to form a plasmid or other closed structure.

Regardless of whether source DNA has the same or a different structure as compared to the enzymatically methylated DNA, the structure of the source DNA may affect the methylation levels or the methylation pattern. The structure of the source DNA is particularly likely to affect whether a specific CpG site is methylated at all or the CpG site specific methylation level because DNA structure may affect the degree to which the CpG site is exposed and thus available for methylation. For example, source DNAs having identical or highly similar sequences are often enzymatically methylated in different patterns depending on whether the source DNA is in a plasmid, other closed, or linear form prior to enzymatic methylation. Supercoiling of source DNA in particular may also affect exposure of CpG sites to the methylase enzyme and, thus, enzymatic methylation. A highly supercoiled source DNA may have lower methylation levels of all three types after enzymatic methylation as compared to a linear source DNA with the same sequence. A highly supercoiled source DNA may have higher levels of methylation in more prominent CpG sites and less methylation in less prominent CpG sites.

The degree to which a specific CpG site is exposed for methylation in source DNA may also be affected by the presence or absence of other DNA-associated elements, such as proteins located over or near the CpG site.

Source DNA may be formed or treated to have a specific structure or DNA-associated elements prior to enzymatic methylation. For example, source DNA from a host cell may be treated to remove all associated proteins or to eliminate supercoiling.

Source DNA, once suitably prepared, if needed, is then incubated with a methylation enzyme or a combination of methylation enzymes able to methylate CpG sites in a reaction sample, along with an enzymatic substrate in an amount sufficient to allow extracellular methylation of at least one CpG site on the source DNA by the methylation enzyme or enzymes. The methylation enzyme or enzymes may be isolated. The incubation may occur in a cell-free environment, but in any event, the methylation of source DNA to produce enzymatically methylated DNA is extracellular—i.e. it does not take place within a cell. Some methylation enzymes may, nevertheless, be associated with co-enzymes or other factors that assist with enzymatic activity. In some embodiments, particularly where source DNA is produced using artificial synthesis, synthesis of the source DNA and enzymatic methylation may occur concurrently or take place within the same reaction sample. Any or all of the types of methylation levels and/or methylation patterns may vary depending upon the methylation enzyme or combination of methylation enzymes used. In some embodiments, at least two methylation enzymes are used for enzymatic methylation. More specifically, at least two different bacterial methylation enzymes may be used, at least two different mammalian methylation enzymes may be used, or a combination of at least one bacterial methylation enzyme and at least one mammalian methylation enzyme may be used.

Suitable methylation enzymes include DNA methyltransferases. Specific DNA methyltransferases that methylate CpG sites include a bacterial methyltransferase, in particular M.SssI methyltransferase, particularly that derived from a Spiroplasma species, M.MpeI methyltransferase, particularly that derived from Mycoplasma penetrans, AluI methyltransferase, HaeIII methyltransferase, HhaI methyltransferase, HpaII methyltransferase, and MspI methyltransferase, and a mammalian DNA methyltransferase (DNMT), in particular DNMT1, DNMT2, DNMT3a, and DNMT3b. As mentioned above, DNMT3a and DNMT3b may also methylation C sites that are non-CpG sites. Other methyltransferases that methylate non-CpG sites include BamHI methyltransferase, Dam methyltransferase, EcoGII methyltransferase, EcoRI methyltransferase, GpC methyltransferase (M.CviPI), MspI methyltransferase, and TaqI methyltransferase.

Low-temperature active enzymes, such as those suitable to achieve a target methylation level within 24 hours at temperatures in a range between 0° C. and 25° C., or above 0° C., but at 25° C. or below, may be used in some embodiments. High-temperature active enzyems, such as those suitable to achieve a target methulation level with 24 hours at temperatures between 45° C. and 100° C., or at 45° C. to below 100° C., may be used in some embodiments.

The methylation enzyme or enzymes, particularly if M.SssI, may be present in an amount of at least about 0.1U, about 0.25U, about 0.5U, about 1.0U, about 1.5U, about 2.0U, about 2.5U, about 3.0U, about 2.5U, about 4U, about 4.5U, about 5U, about 6U, or about 8U, or in an amount of about 0.1U, about 0.25U, about 0.5U, about 1.0U, about 1.5U, about 2.0U, about 2.5U, about 3.0U, about 2.5U, about 4U, about 4.5U, about 5U, about 6U, or about 8U to about 10U or about 20U per enzyme. In other embodiments, the enzyme may be present in these amounts per mg of DNA to be methylated.

Low-temperature active enzymes, such as those suitable to achieve a target methylation level within 24 hours at temperatures in a range between 0° C. and 25° C., or above 0° C., but at 25° C. or below, may be used in some embodiments. High-temperature active enzyems, such as those suitable to achieve a target methulation level with 24 hours at temperatures between 45° C. and 100° C., or at 45° C. to below 100° C., may be used in some embodiments.

The suitable enzymatic substrate can be any methyl donor compatible with the methylation enzyme, but typically it will be S-adenosyl methionine (SAM). SAM may be present in any amount, but most typically, particularly when used with M.SssI methyltransferase, will be present in a concentration of at least one of the following concentrations or in a range from any one of to another of any two concentrations selected from about 1 μM, about 2 μM, about 4 μM, about 5 μM, about 8 μM, about 10 μM, about 20 μM, about 40 μM, about 50 μM, about 100 μM, about 125 μM, about 150 μM, about 160 μM, about 170 μM, about 175 μM, about 200 μM, and about 250 μM. In particular, the enzymatic substrate may be present in a range from about 150 μM and about 170 μM or about 200 μM. In some embodiments a set methylation level of a methylation level type or a set methylation pattern may be achieved by regulating the of concentration of SAM present. For instance, lower concentrations of SAM, such as 10 μM or less, may lead to only partial methylation of the enzymatically methylated DNA.

The source DNA is incubated with the methylation enzyme or enzymes and enzymatic substrate in the reaction sample for a time and under conditions suitable to achieve at least one set level of methylation of a methylation level type or a set methylation pattern in the enzymatically methylated DNA.

The incubation time and incubation conditions may be varied to achieve the set level or levels of methylation and/or methylation pattern. Incubation conditions include amount of methylation enzyme or enzymes, identity of methylation enzyme or enzymes, relative amount of methylation enzymes of two or more are present, concentration or total amount of enzymatic substrate, total number of CpG sites within the source DNA or a region thereof, number of CG sequences within at least one CpG site, temperature, the presence or concentration of magnesium ion or other divalent cation, and structure and methylation enzymatic accessibility of source DNA or a region thereof (e.g. the topological state of the DNA).

For example, other conditions being equal, a longer incubation time generally yields higher levels of methylation of all types. Incubation time may be in a range from any one of to another of any two of the following time periods: about 1 minute, about 5 minutes, about 10 minutes, about 15 minutes, about 20 minutes, about 30 minutes, about 45 minutes, about 1 hour, about 2 hours, about 12 hours, or about 24 hours.

In addition, other conditions being equal, a higher amount of methylation enzyme also yields higher levels of methylation of all types.

Further, the concentration or total amount of enzymatic substrate in the reaction sample may influence levels of methylation of all types, with greater concentration of substrate typically yielding higher levels of methylation, all other conditions being equal. However, it is possible to limit the methylation levels of all types, regardless of substrate concentration, by limiting the total amount of substrate as compared to the total amount of source DNA present in the reaction sample. Source DNA may only be methylated until the supply of enzymatic substrate is exhausted.

Additionally, the total number of CpG sites and CG sequences with CpG sites within the source DNA or a region of the source DNA may affect methylation levels, methylation patterns, and the potential to methylate a specific CpG site as well as its CpG site specific methylation level as compared to what would be achievable with source DNA of a similar length and structure, but with a different number of CpG sites or CG sequences within CpG sites.

The level of methylation of all types may also be controlled by temperature, but for each type may be less sensitive to temperature than methylation level achieved using host cells, particularly bacterial cells. The reacting sample may be formed using liquid components at one temperature, such as about 25° C. or in a range from about 20° C. to about 30° C., then incubated at a higher incubation temperature, such as at one of the following temperatures or in a range from any one of to another of any two temperatures selected from: about 25° C., about 30° C., about 32° C., about 33° C., about 34° C., about 35° C., about 36° C., about 37° C., about 38° C., about 39° C., about 40° C., and about 45° C. Higher or lower temperatures may be used for low-temperature or high-temperature active methylation enzymes.

In addition, magnesium ion may be added to the reaction sample, in which case, the concentration of magnesium ion also affects the level of methylation of all types of the enzymatically methylated DNA, as well as methylation patterns. Methylation patterns in particular may be affected. In general, higher concentrations of magnesium ion cause enzymatic methylation in patterns more consistent with distributive enzymatic activity and also decrease the amount of fully methylated DNA. The concentration of magnesium ion in the reaction sample may be at least one of the following concentrations or in a range from any one of to another of two concentrations selected from about 1 mM, about 2.5 mM, about 5 mM, about 10 mM, about 15 mM, about 20 mM, about 50 mM, about 100 mM, and about 200 mM. A magnesium ion concentration of 40 mM in the reaction sample may inhibit enzymatic methylation. Magnesium ion may be provided as a magnesium salt, such as magnesium acetate or magnesium chloride. Magnesium ion may effectively be removed from a reaction sample by adding ethylenediaminetetraacetic acid (EDTA) to the sample. Similar results may be obtained with other divalent cations, such as calcium ion.

Enzymatic methylation may occur with the enzyme acting in a processive or distributive manner. When the methylation enzyme acts in a processive manner, it tends to remain associated with a DNA molecule and catalyze many methylation reactions before releasing the DNA molecule. When the methylation enzyme acts in a distributive manner, it tends to associate with the DNA molecule to perform only a few methylation reactions or sometimes even only a single methylation reaction, then release the molecule. For any given methylation enzyme, the enzyme will, on average, perform more methylation reactions before releasing a DNA molecule when operating in a processive manner than when operating in a distributive manner. Any of the reaction conditions mentioned herein can influence whether the enzyme acts primarily in a processive or distributive manner.

Enzymatically methylated DNA may be further processed. For example, it may be cleaved by restriction enzymes linearize the DNA or to excise linear fragments, which may form therapeutics on their own or be ligated into other DNA structures, such as plasmids or other closed DNA structures, such as a covalently-closed linear double stranded DNA structure. For example, linear DNA, whether synthesized as linear or linearized from a plasmid, may then be converted to a circularized or other shape, such as a plasmid, for example by ligation. Enzymatically methylated DNA may also be treated to alter its structure, for example by the addition of proteins or other conformation-changing agents. Enzymatically methylated DNA may further be treated or formed from source DNA containing modified bases to reduce the chances of a cellular immune response to the enzymatically methylated DNA. Methylation may also increase the expression time of a transgene in a mammalian cell which may be useful, for example, in gene therapy.

Of the various enzymatic methylation conditions discussed herein, the incubation time and amount of methylation enzyme or enzymes are very likely to influence methylation levels of all types. The structure of source DNA is very likely to influence methylation patterns and methylation of specific CpG sites.

The reaction solution may contain other components helpful to stabilize one or more reactants or to maintain a pH at which the methylation enzyme or enzymes is/are active, such as potassium acetate, Tris acetate, and bovine serum albumin.

After incubation, the temperature of the reaction sample may be raised to quenching temperature at which the methylation enzyme or enzymes can no longer methylate DNA. The quenching temperature is preferably at least as high as the temperature at which the methylation enzyme or enzymes is/are irreversibly denatured, such that DNA methylation will no longer occur even after the reaction sample is cooled. The quenching temperature is also preferably not so high that the DNA itself is damaged or denatured. For example, the quenching temperature may be at least 50° C., about 55° C., about 60° C., or about 65° C., or in a range from about 50° C., about 55° C., about 60° C., about 65° C., about 70° C., about 75° C., about 76° C., about 80° C., about 85° C., about 90° C., or about 95° C., to about 100° C. In some embodiments, the method does not include a quenching step.

### Pharmaceutical Compositions Containing Enzymatically Methylated DNA

The present disclosure further provides compositions including enzymatically methylated DNA as described herein. Such compositions may be pharmaceutical compositions including the enzymatically methylated DNA and a pharmaceutically acceptable carrier, excipient, or diluent.

In some embodiments, the enzymatically methylated DNA may be present in the pharmaceutical composition without any delivery vehicle or carrier.

In some embodiments, a composition the enzymatically methylated DNA may be coupled to a delivery vehicle or carrier suitable for administration to the patient. Exemplary vehicles or carriers include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. Nanomed Nanobiotechnol. 11 (2): e1530 (2019), the delivery vehicle and carrier portions of which are incorporated by reference herein). Carriers may also include a viral-based carrier.

The pharmaceutical composition may be suitable for injection, such as a liquid pharmaceutical composition, which may, in particular be a solution or a suspension. The liquid pharmaceutical composition may further include: water, saline solution, preferably physiological saline, Ringer's solution, isotonic sodium chloride, fixed oils such as synthetic mono or diglycerides which may serve as the solvent or suspending medium, polyethylene glycols, glycerin, propylene glycol, glycerols, injectable organic esters or other solvents or formulation carriers; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

The pharmaceutical composition may also include physiologically acceptable compounds that act, for example, to stabilize or to increase absorption of the enzymatically modified DNA. Such physiologically acceptable compounds include, for example, carbohydrates, such as glucose, sucrose or dextrans, antioxidants, such as ascorbic acid or glutathione, chelating agents, low molecular weight proteins or other stabilizers or excipients.

The pharmaceutical composition may be intended for topical administration and may include a solution, emulsion, ointment or gel base. If intended for transdermal administration, the composition may include a transdermal patch or iontophoresis device.

The pharmaceutical compositions may be prepared by methodologies well known in the pharmaceutical art. For example, a composition intended to be administered by injection can be prepared by combining a composition that comprises enzymatically methylated DNA as described herein, either with or without a delivery vehicle or carrier, and optionally, one or more of salts, buffers and/or stabilizers, with sterile, distilled water so as to form a solution. A surfactant may be added to facilitate the formation of a homogeneous solution or suspension.

The composition may include more than one different enzymatically methylated DNA according to the present disclosure. For example, it may include an enzymatically methylated DNA with a stimulating methylation level and an enzymatically methylated DNA with an attenuating methylation level, particularly containing different genes. It may also include two or more different enzymatically methylated DNA molecules both with stimulating methylation levels or two or more different enzymatically methylated DNA molecules, both with attenuating methylation levels, particularly containing different genes in both cases.

In one embodiment, the composition may necessarily include enzymatically methylated DNA containing a gene encoding a pro-apoptotic protein, more particularly BAX. In a more specific embodiment, the pro-apoptotic protein may be the only protein encoded by enzymatically methylated DNA in the composition. In another more specific embodiment, enzymatically methylated DNA may further encode at least one of a tolerance-inducing protein or a determinant protein. In another more specific embodiment, one enzymatically methylated DNA may encode the pro-apoptotic protein and a second enzymatically methylated DNA may encode the tolerance-inducing protein or determinant protein. In an even more specific embodiment, the second enzymatically methylated DNA may encode both a tolerance-inducing protein and a determinant protein. In another specific embodiment, one enzymatically methylated DNA may encode the pro-apoptotic protein and tolerance-inducing protein and as second enzymatically methylated DNA may encode a determinant protein. In yet another specific embodiment, the same enzymatically methylated DNA may encode both a pro-apoptotic protein and a determinant protein. In a more specific embodiment, the same enzymatically methylated DNA may further encode a tolerance-inducing protein. In a different more specific embodiment, a second different enzymatically methylated DNA may encode a tolerance-inducing protein.

In any of these embodiments in which two different enzymatically methylated DNA molecules are present, one may have a stimulating methylation level while the other has an attenuating methylation level. In particular, the enzymatically methylated DNA encoding the pro-apoptotic protein may have a stimulating methylation level and the enzymatically methylated DNA encoding the determinant protein may have an attenuating methylation level.

The pharmaceutical composition may further include another therapeutic, such as, for example, an inflammatory or anti-inflammatory compound or a chemotherapeutic that augments the therapeutic effect of the enzymatically methylated DNA.

### Methods Using Enzymatically Methylated DNA

Enzymatically methylated DNA as described herein may be used to modulate the immune response of a patient and, thereby, treat a condition that the patient suffers. “Treating” or providing a “therapy” for a condition, as used herein, refers to the alleviation or elimination of at least one symptom of the condition. A “therapeutically effective amount” of an enzymatically methylated DNA or a composition containing an enzymatically methylated DNA is an amount sufficient to cause such alleviation or elimination of at least one symptom of the condition.

In particular, enzymatically methylated DNA that has a stimulating methylation level is generally likely to cause a targeted upregulation of an immune response or a reactive immune response to the antigen and may be used for that purpose. For example, a cancer antigen may be administered in enzymatically methylated DNA with a stimulating methylation level. Enzymatically methylated DNA that has an attenuating methylation level is likely to cause a targeted downregulation of an immune response or a tolerogenic immune response to the antigen and may be used for that purpose. For example, an allergen, autoantigen, or donor antigen or sequestered tissue specific antigen may be administered in enzymatically methylated DNA with an attenuating methylation level. However, combinations of enzymatically methylated DNA with a stimulating methylation level and enzymatically methylated DNA with an attenuating methylation level may also be used, as may enzymatically methylated DNA with certain methylation patterns or certain CpG site specific methylation levels in order to achieve appropriate modulation of the immune response in a patient.

In addition, promoter methylation is typically associated with reduced expression of a protein under control of the promotor. Accordingly, in some embodiments, promoter methylation may be adjusted to affect expression of the protein under control of the promoter. For example, CpG site specific methylation levels for CpG sites within the promoter may have a stimulating methylation level if the promoter controls expression of a pro-apoptotic protein, a tolerance-inducing protein, or a cancer antigen to induce higher expression of the protein so that apoptosis, an antigen-specific tolerogenic immune response, or an antigen-specific reactive immune response, respectively, occurs. Conversely, CpG site specific methylation levels for CpG sites within the promoter may have an attenuating methylation level if the promoter controls expression on an allergen, autoantigen, or donor antigen or sequestered tissue specific antigen to control express of the protein and maintain it at a tolerogenic level.

Changes in methylation levels of any type or methylation pattern are also sometimes associated with disease. Accordingly, in some embodiments, the pro-apoptotic protein or determinant protein may be selected to match a protein associated with such a disease and the methylation levels or any type or methylation pattern in the enzymatically methylated DNA encoding the protein may be adjusted to compensate for any aberrant methylation status associated with the disease.

In some embodiments enzymatically methylated DNA containing a gene encoding a pro-apoptotic protein may be administered to downregulate the immune response to or induce an antigen-specific tolerogenic response to: a determinant protein encoded by same enzymatically methylated DNA or a co-administered second, different enzymatically methylated DNA, a determinant protein located in the area of administration, or a determinant protein not located in the area of administration. The determinant protein may be an allergen, an autoantigen, or a donor antigen or sequestered tissue specific antigen.

In some embodiments, enzymatically methylated DNA may be administered to upregulate the immune response to a determinant protein or to cause an antigen-specific reactive immune response to a determinant protein, such as a cancer antigen, encoded by the enzymatically methylated DNA.

Autoimmune conditions that may be treated by administering a therapeutically effective amount of an enzymatically methylated DNA include: an alopecia, such as alopecia areata, alopecia totalis, alopecia universalis, or alopecia ophiasis, rejection of solid organ transplants, graft versus host disease, host versus graft disease, autoimmune hepatitis, vitiligo, diabetes mellitus type 1, Addison's Disease, Graves' disease, Hashimoto's thyroiditis, other autoimmune thyroiditis, multiple sclerosis, polymyalgia rheumatica, Reiter's syndrome, Crohn's disease, Goodpasture's syndrome, Gullain-Barre syndrome, lupus nephritis, rheumatoid arthritis, systemic lupus erythematosus, Wegener's granulomatosis, celiac disease, dermatomyositis, eosinophilic fasciitis, idiopathic thrombocytopeniaurpura, Miller-Fisher syndrome, myasthenia gravis, pemphigus vulgaris, pernicious anemia, polymyositis, primary biliary cirrhosis, psoriasis, psoriatic arthritis, rheumatoid arthritis, Sjogren's syndrome, and autoimmune hepatitis.

Allergies that may be treated by administering a therapeutically effective amount of an enzymatically methylated DNA include allergies to any protein allergen, including any of the allergens identified herein, or haptens that cause an allergic response once combined with certain host cellular elements. Secondary effects of allergies, such as asthma, may also be treated.

Cancers that may be treated by administering a therapeutically effective amount of an enzymatically methylated DNA include: Burkitt lymphoma, neuroblastoma, melanoma, osteosarcoma, renal cell carcinoma, breast cancer, prostate cancer, lung carcinoma, colon cancer, germ cell tumors, ovarian cancer, and hepatocellular carcinoma.

Transplant rejections that may be treated by administering a therapeutically effective amount of an enzymatically methylated DNA include rejections of any organ or tissue transplants primarily or secondarily mediated by an immune response to a donor antigen or sequestered tissue specific antigen.

In a specific embodiment, an allergy, an autoimmune disease, or transplant rejection maybe treated by administering a therapeutically effective amount of: (i) a combination of an enzymatically methylated DNA having a stimulating methylation level and encoding BAX and an enzymatically methylated DNA having an attenuating methylation level and encoding both BAX and the allergen, autoantigen, or donor antigen or sequestered tissue specific antigen, or (ii) a combination of an enzymatically methylated DNA having a stimulating methylation level and encoding BAX, such as pSV40-hBAX-BLa (FIG. 3) or pSV40-sGAD55+hBAX-BLa (FIG. 17), and an enzymatically methylated DNA having an attenuating methylation level and encoding the allergen, autoantigen, or donor antigen, such as pSV40-sGAD55. A tolerance-inducing protein may also be encoded by either of the plasmids in these combinations or by a third enzymatically methylated plasmid having a stimulating methylation level.

DNA combinations of this embodiment and other embodiments including enzymatically methylated DNA encoding the allergen, autoantigen, or donor antigen or sequestered tissue specific antigen may induce a tolerogenic immune response in in a patient administered the DNA combination. Unmethylated CpG sites tend to activate toll-like receptor-9 (TLR9) in mammals, which induces immune response useful in eliminating bacteria and viruses. The tolerance effect may be wholly or partially dependent on modulation of TLR9 activation by enzymatically methylated DNA.

In embodiments where the patient is administered two different enzymatically methylated DNAs, the two different DNAs may be administered together, in a single pharmaceutical composition, or separately in separate pharmaceutical compositions. In either case, the different enzymatically methylated DNAs may be administered in any of the following ratios, or in range from any one to another of the following ratios: about 10:1, about 9:1, about 8:1, about 7:1, about 6:1, about 5:1, about 4:1, about 3:1, about 2:1, or about 1:1.

In the embodiment where a first enzymatically methylated DNA having an antigen-specific attenuating methylation level and encoding GAD, sGAD (a secreted form of GAD), or a functional fragment thereof, such as pSV40-sGAD55 (FIG. 2A and FIG. 2B), and a second enzymatically methylated DNA having a stimulating methylation level and encoding BAX, such as pSV40-hBAX-BLa or pSV-40-sGAD55+hBAX-BLa (FIG. 17) (FIG. 3) are administered, the ratio of the first enzymatically methylated DNA to the second enzymatically methylated DNA may be in a range from about 1:1 and 3:1 or about 1:4 and 3:1, particularly about 2:1.

Compositions containing other therapeutics may also be administered to the patient. Additional therapeutics may include an immunosuppressant agent such as a corticosteroid, a glucocorticoid, a cyclophosphamide, a 6-mercaptopurine (6-MP), an azathioprine (AZA), methotrexate cyclosporine, mycophenolate mofetil (MMF), mycophenolic acid (MPA), tacrolimus (FK506), sirolimus ([SRL] rapamycin), everolimus (Certican), mizoribine, leflunomide, deoxyspergualin, brequinar, azodicarbonamide, a vitamin D analog, such as MC1288 or bisindolylmaleimide VIII, antilymphocyte globulin, antithymocyte globulin (ATG), an anti-CD3 monoclonal antibody, (Muromonab-CD3, Orthoclone OKT3), an anti-interleukin (IL)-2 receptor (anti-CD25) antibody, (Daclizumab, Zenapax, basiliximab, Simulect), an anti-CD52 antibody, (Alemtuzumab, Campath-IH), an anti-CD20 antibody (Rituximab, Rituxan), an anti-tumor necrosis factor (TNF) reagent (Infliximab, Remicade, Adalimumab, Humira), or an LFA-1 inhibitor (Efalizumab, Raptiva).

Additional therapeutics may also include a chemotherapeutic, such as small molecule chemotherapeutics as well as large molecule chemotherapeutics, particularly a checkpoint inhibitor, an alkylating agent, an antimetabolite, an alkaloid, particularly a plant alkaloid, an antitumor antibiotic, or an antitumor antibody.

In specific embodiments of the present disclosure, the enzymatically methylated DNA is administered by injection, particularly intradermal injection. Injection may be at an injection site where the allergen, autoantigen, cancer antigen, or donor antigen or sequestered tissue specific antigen is present. Injection may also be at an injection site where the allergen, autoantigen, cancer antigen, or donor antigen or sequestered tissue specific antigen is not present, or where presence of the allergen, autoantigen, cancer antigen, or donor antigen or sequestered tissue specific antigen is not known.

The enzymatically methylated DNA need only be administered until the desired therapeutic effect has been achieved. Accordingly, in many embodiments, the enzymatically methylated DNA may be administered, then the patient monitored for alleviation of at least one symptom of the condition or for markers of the desired modulation of immune response in the patient. Often the enzymatically methylated DNA may need only be administered a single time because a single administration will be sufficient to modulate the immune response and, for example, cause tolerance of an autoantigen, an allergen or a donor antigen or sequestered tissue specific antigen or cause the body to recognize and respond to a tumor antigen.

Other Methods of Producing and Using pSV40-hBAX-BLa and pSV40-sGAD55-BLa

pSV40-hBAX-BLa, pSV40-sGAD55-BLa, and pSV40-sGAD55+hBAX-BLa may be produced in E. coli, then purified prior to administration to subjects. DNA produced in E. coli may be methylated at sites containing a CpG sequence, i.e., 5′-C-phosphate-G-3′, where cytosine and guanine are separated by one phosphate group. If pSV40-nBAX-BLa or pSV40-sGAD55BLa is produced in E. coli that lack the gene for a CpG methylation enzyme, then the DNA will be methylated only at low levels, with typically less than 3% of available CpG sites having a methyl group. If pSV40-BAX-BLa or pSV40-sGAD55-BLa is produced in bacteria having a methyltransferase gene, then higher levels of methylation may be obtained depending on the bacterial growth conditions. Bacterial growth conditions may also affect methylation patterns.

In addition, either pSV40-hBAX-BLa or pSV40-sGAD55-BLa may first be produced in a bacteria, such as E. coli, then enzymatically methylated in an extracellular environment in vitro by incubating the plasmid with a methyltransferase enzyme and methyl source under conditions in which the methyltransferase enzyme is active. Levels, specificity, and patterns of methylation may be controlled by methylation conditions. Enzymatic methylation may be as disclosed herein.

pSV40-BAX-BLa with greater methylation than the default levels achieved in E. coli, such as greater than 3%, may be referred to as mpSV40-BAX-BLa and pSV40-sGAD55-BLa with greater methylation than the default levels achieved in E. coli, such as greater than 3%, may be referred to as mpSV40-sGAD55-BLa.

The present disclosure provides mpSV40-hBAX-BLa, mpSV40-hBAX-BLa, pSV40-sGAD55-BLa, mpSV40-sGAD55-BLa, pSV40-sGAD55+hBAX-BLA, and mpSV40-sGAD55+hBAX-BLA. Each of these plasmids may be isolated.

The present disclosure additionally provides a pharmaceutical composition containing at least one of pSV40-hBAX-BLa, mpSV40-hBAX-BLa, pSV40-sGAD55-BLa, mpSV40-sGAD55-BLa, pSV40-sGAD55-hBAX-BLa, or mpSV40-sGAD55-hBAX-BLA in a pharmaceutically acceptable carrier, excipient or diluent. The plasmids may be provided separately in separate pharmaceutical compositions, or together in a single pharmaceutical composition.

In a specific embodiment, the present disclosure provides mpSV40-hBAX-BLa in a pharmaceutically acceptable carrier, excipient, or diluent.

In another specific embodiment, the present disclosure provides both mpSV40-hBAX-BLa and mpSV40-sGAD55-BLa in a pharmaceutically acceptable carrier. More specifically, the ratio of both mpSV40-hBAX-BLa to mpSV40-sGAD55-BLa may be in a range from about 1:1 and 3:1 or 1:4 and 3:1, particularly about 2:1.

In some embodiments, the mpSV40-hBAX-BLa may be methylated at a lower level than mpSV40-sGAD55-BLa. In particular, mpSV40-hBAX-BLa may be methylated such that methylation of the SV40 promoter does not substantially interfere with BAX expression, such that apoptosis and/or dendritic cell recruitment occur.

In some embodiments, the mpSV40-sGAD55-BLa may be methylated at a higher level than mpSV40-hBAX-BLa. In particular, mpSV40-sGAD55-BLa may be methylated such that methylation of the SV40 promoter controls expression of sGAD to an amount that induces only a tolerogenic immune response, not a reactive immune response.

In some embodiments, pSV40-sGAD55+hBAX-BLa or m pSV40-sGAD55+hBAX-BLa is provided in a pharmaceutical composition with pSV40-hBAX-BLa, mp SV40-hBAX-BLa, pSV40-sGAD55-BLa, or mpSV40-sGAD55-BLa.

In some embodiments, compositions are provided in which the plasmid is in any composition pharmaceutical formulation and used in any applicable method as disclosed above for enzymatically methylated plasmids, regardless of how it is methylated (e.g. a bacterially methylated plasmid may be provided in the compositions disclosed for enzymatically methylated plasmids and used in the same manner as enzymatically methylated plasmids).

Table 1 provides sequences for plasmids according to the present disclosure as well as identified functional elements and restriction sites thereof.

| SEQ ID | | |
|---|---|---|
| NO: | Description | Sequence |
| 1 | pSV40-hBAX- | tcgcgatgta cgggccagat atacgcgttc tgtggaatgt |
| | BLa | gtgtcagtta gggtgtggaa agtccccagg ctccccagca |
| | | ggcagaagta tgcaaagcat gcatctcaat tagtcagcaa |
| | | ggaaagtccc caggctcccc agcaggcaga agtatgcaaa |
| | | gcatgcatct caattagtca gcaaccatag tcccgcccct |
| | | aactccgccc atcccgcccc taactccgcc cagttccgcc |
| | | cattctccgc cccatggctg actaattttt tttatttatg cagaggccga |
| | | ggccgcctcg gcctctgagc tattccagaa gtagtgaaga |
| | | ggcttttttg gaggcctagg cttttgcaaa aagctccgga |
| | | tcgatcctga gaacttcagg gtgagtttgg ggacccttga ttgttctttc |
| | | tttttcgcta ttgtaaaatt catgttatat ggagggggca aagttttcag |
| | | ggtgttgttt agaatgggaa gatgtccctt gtatcaccat |
| | | ggaccctcat gataattttg tttctttcac tttctactct gttgacaacc |
| | | attgtctcct cttattttct tttcattttc tgtaactttt tcgttaaact |
| | | ttagcttgca tttgtaacga atttttaaat tcacttttgt ttatttgtca |
| | | gattgtaagt actttctcta atcacttttt tttcaaggca atcagggtat |
| | | attatattgt acttcagcac agttttagag aacaattgtt ataattaaat |
| | | gataaggtag aatatttctg catataaatt ctggctggcg tggaaatatt |
| | | cttattggta gaaacaacta catcctggtc atcatcctgc ctttctcttt |
| | | atggttacaa tgatatacac tgtttgagat gaggataaaa |
| | | tactctgagt ccaaaccggg cccctctgct aaccatgttc |
| | | atgccttctt ctttttccta cagctcctgg gcaacgtgct ggttattgtg |
| | | ctgtctcatc attttggcaa agaattgtaa tacgactcac |
| | | tatagggcga attcgcggtg atggacgggt ccggggagca |
| | | gcccagaggc ggggggccca ccagctctga gcagatcatg |
| | | aagacagggg cccttttgct tcagggtttc atccaggatc |
| | | gagcagggcg aatggggggg gaggcacccg agctggccct |
| | | ggacccggtg cctcaggatg cgtccaccaa gaagctgagc |
| | | gagtgtctca agcgcatcgg ggacgaactg gacagtaaca |
| | | tggagctgca gaggatgatt gccgccgtgg acacagactc |
| | | cccccgagag gcctttttcc gagtggcagc tgacatgttt |
| | | tctgacggca acttcaactg gggccgggtt gtcgcccttt |
| | | tctactttgc cagcaaactg gtgctcaagg ccctgtgcac |
| | | caaggtgccg gaactgatca gaaccatcat gggctggaca |
| | | ttggacttcc tccgggagcg gctgttgggc tggatccaag |
| | | accagggtgg ttgggacggc ctcctctcct actttgggac |
| | | gcccacgtgg cagaccgtga ccatctttgt ggcgggagtg |
| | | ctcaccgcct cactcaccat ctggaagaag atgggctgag |
| | | aattctgcag atatccagca cagtggcggc cgctcgagtc |
| | | tagagggccc gtttaaaccc gctgatcagc ctcgactgtg |
| | | ccttctagtt gccagccatc tgttgtttgc ccctcccccg tgccttcctt |
| | | gaccctggaa ggtgccactc ccactgtcct ttcctaataa |
| | | aatgaggaaa ttgcatcgca ttgtctgagt aggtgtcatt |
| | | ctattctggg gggtggggtg gggcaggaca gcaaggggga |
| | | ggattgggaa gacaatagca ggcatgctgg ggatgcggtg |
| | | ggctctatgg cttctactgg gcggttttat ggacagcaag |
| | | cgaaccaccg gtacccgggc ccatggcgcg gaacccctat |
| | | ttgtttattt ttctaaatac attcaaatat gtatccgctc atgagacaat |
| | | aaccctgata aatgcttcaa taatattgaa aaaggaagag |
| | | tatgagtatt caacatttcc gtgtcgccct tattcccttt tttgcggcat |
| | | tttgccttcc tgtttttgct cacccagaaa cgctggtgaa |
| | | agtaaaagat gctgaagatc agttgggtgc acgagtgggt |
| | | tacatcgaac tggatctcaa cagcggtaag atccttgaga |
| | | gttttcgccc cgaagaacgt tttccaatga tgagcacttt taaagttctg |
| | | ctatgtggcg cggtattatc ccgtattgac gccgggcaag |
| | | agcaactcgg tcgccgcata cactattctc agaatgactt |
| | | ggttgagtac tcaccagtca cagaaaagca tcttacggat |
| | | ggcatgacag taagagaatt atgcagtgct gccataacca |
| | | tgagtgataa cactgcggcc aacttacttc tgacaacgat |
| | | cggaggaccg aaggagctaa ccgctttttt gcacaacatg |
| | | ggggatcatg taactcgcct tgatcgttgg gaaccggagc |
| | | tgaatgaagc cataccaaac gacgagcgtg acaccacgat |
| | | gcctgtagca atggcaacaa cgttgcgcaa actattaact |
| | | ggcgaactac ttactctagc ttcccggcaa caattaatag |
| | | actggatgga ggcggataaa gttgcaggac cacttctgcg |
| | | ctcggccctt ccggctggct ggtttattgc tgataaatct |
| | | ggagccggtg agcgtgggtc tcgcggtatc attgcagcac |
| | | tggggccaga tggtaagccc tcccgtatcg tagttatcta |
| | | cacgacgggg agtcaggcaa ctatggatga acgaaataga |
| | | cagatcgctg agataggtgc ctcactgatt aagcattggt |
| | | aactgtcaga ccaagtttac tcatatatac tttagattga tttaaaactt |
| | | catttttaat ttaaaaggat ctaggtgaag atcctttttg ataatctcat |
| | | gaccaaaatc ccttaacgtg agttttcgtt ccactgagcg |
| | | tcagaccccg tactagtact cgagctcgcg aagaaaagat |
| | | caaaggatct tcttgagatc ctttttttct gcgcgtaatc tgctgcttgc |
| | | aaacaaaaaa accaccgcta ccagcggtgg tttgtttgcc |
| | | ggatcaagag ctaccaactc tttttccgaa ggtaactggc |
| | | ttcagcagag cgcagatacc aaatactgtt cttctagtgt |
| | | agccgtagtt aggccaccac ttcaagaact ctgtagcacc |
| | | gcctacatac ctcgctctgc taatcctgtt accagtggct |
| | | gctgccagtg gcgataagtc gtgtcttacc gggttggact |
| | | caagacgata gttaccggat aaggcgcagc ggtcgggctg |
| | | aacggggggt tcgtgcacac agcccagctt ggagcgaacg |
| | | acctacaccg aactgagata cctacagcgt gagctatgag |
| | | aaagcgccac gcttcccgaa gggagaaagg cggacaggta |
| | | tccggtaagc ggcagggtcg gaacaggaga gcgcacgagg |
| | | gagcttccag ggggaaacgc ctggtatctt tatagtcctg |
| | | tcgggtttcg ccacctctga cttgagcgtc gatttttgtg atgctcgtca |
| | | ggggggcgga gcctatggaa aaacgccagc aacgcggcct |
| | | ttttacggtt cctggccttt tgctggcctt ttgctcacat gttcttgctg ct |
| 2 | pSV40-sGAD55- | tcgcgatgta cgggccagat atacgcgttc tgtggaatgt |
| | BLa | gtgtcagtta gggtgtggaa agtccccagg ctccccagca |
| | | ggcagaagta tgcaaagcat gcatctcaat tagtcagcaa |
| | | ggaaagtccc caggctcccc agcaggcaga agtatgcaaa |
| | | gcatgcatct caattagtca gcaaccatag tcccgcccct |
| | | aactccgccc atcccgcccc taactccgcc cagttccgcc |
| | | cattctccgc cccatggctg actaattttt tttatttatg cagaggccga |
| | | ggccgcctcg gcctctgagc tattccagaa gtagtgaaga |
| | | ggcttttttg gaggcctagg cttttgcaaa aagctccgga |
| | | tcgatcctga gaacttcagg gtgagtttgg ggacccttga ttgttctttc |
| | | tttttcgcta ttgtaaaatt catgttatat ggagggggca aagttttcag |
| | | ggtgttgttt agaatgggaa gatgtccctt gtatcaccat |
| | | ggaccctcat gataattttg tttctttcac tttctactct gttgacaacc |
| | | attgtctcct cttattttct tttcattttc tgtaactttt tcgttaaact |
| | | ttagcttgca tttgtaacga atttttaaat tcacttttgt ttatttgtca |
| | | gattgtaagt actttctcta atcacttttt tttcaaggca atcagggtat |
| | | attatattgt acttcagcac agttttagag aacaattgtt ataattaaat |
| | | gataaggtag aatatttctg catataaatt ctggctggcg tggaaatatt |
| | | cttattggta gaaacaacta catcctggtc atcatcctgc ctttctcttt |
| | | atggttacaa tgatatacac tgtttgagat gaggataaaa |
| | | tactctgagt ccaaaccggg cccctctgct aaccatgttc |
| | | atgccttctt ctttttccta cagctcctgg gcaacgtgct ggttattgtg |
| | | ctgtctcatc attttggcaa agaattgtaa tacgactcac |
| | | tatagggcga attcggatcc actagtccag tgtggtggaa |
| | | ttctgcagat atccagcaca gtggcggccg ctcgacggta |
| | | tcgataagct tgatatcgaa ttcgttggga ttttctagaa tgtacaggat |
| | | gcaactcctg tcttgcattg cactaagtct tgcacttgtc |
| | | acaaacagtg cacctactta cgcgtttctc catgcaacag |
| | | acctgctgcc ggcgtgtgat ggagaaaggc ccactttggc |
| | | gtttctgcaa gatgttatga acattttact tcagtatgtg gtgaaaagtt |
| | | tcgatagatc aaccaaagtg attgatttcc attatcctaa tgagcttctc |
| | | caagaatata attgggaatt ggcagaccaa ccacaaaatt |
| | | tggaggaaat tttgatgcat tgccaaacaa ctctaaaata |
| | | tgcaattaaa acagggcatc ctagatactt caatcaactt tctactggtt |
| | | tggatatggt tggattagca gcagactggc tgacatcaac |
| | | agcaaatact aacatgttca cctatgaaat tgctccagta tttgtgcttt |
| | | tggaatatgt cacactaaag aaaatgagag aaatcattgg |
| | | ctggccaggg ggctctggcg atgggatatt ttctcccggt |
| | | ggcgccatat ctaacatgta tgccatgatg atcgcacgct |
| | | ttaagatgtt cccagaagtc aaggagaaag gaatggctgc |
| | | tcttcccagg ctcattgcct tcacgtctga acatagtcat ttttctctca |
| | | agaagggagc tgcagcctta gggattggaa cagacagcgt |
| | | gattctgatt aaatgtgatg agagagggaa aatgattcca |
| | | tctgatcttg aaagaaggat tcttgaagcc aaacagaaag |
| | | ggtttgttcc tttcctcgtg agtgccacag ctggaaccac |
| | | cgtgtacgga gcatttgacc ccctcttagc tgtcgctgac |
| | | atttgcaaaa agtataagat ctggatgcat gtggatgcag |
| | | cttggggtgg gggattactg atgtcccgaa aacacaagtg |
| | | gaaactgagt ggcgtggaga gggccaactc tgtgacgtgg |
| | | aatccacaca agatgatggg agtccctttg cagtgctctg |
| | | ctctcctggt tagagaagag ggattgatgc agaattgcaa |
| | | ccaaatgcat gcctcctacc tctttcagca agataaacat |
| | | tatgacctgt cctatgacac tggagacaag gccttacagt |
| | | gcggacgcca cgttgatgtt tttaaactat ggctgatgtg |
| | | gagggcaaag gggactaccg ggtttgaagc gcatgttgat |
| | | aaatgtttgg agttggcaga gtatttatac aacatcataa |
| | | aaaaccgaga aggatatgag atggtgtttg atgggaagcc |
| | | tcagcacaca aatgtctgct tctggtacat tcctccaagc |
| | | ttgcgtactc tggaagacaa tgaagagaga atgagtcgcc |
| | | tctcgaaggt ggctccagtg attaaagcca gaatgatgga |
| | | gtatggaacc acaatggtca gctaccaacc cttgggagac |
| | | aaggtcaatt tcgtccgcat ggtcatctca aacccagcgg |
| | | caactcacca agacattgac ttcctgattg aagaaataga |
| | | acgccttgga caagatttat aataaccttg ctcaccaagc |
| | | tgttcacttc ttcgagtcta gagggcccgt ttaaacccgc |
| | | tgatcagcct cgactgtgcc ttctagttgc cagccatctg ttgtttgccc |
| | | ctcccccgtg ccttccttga ccctggaagg tgccactccc |
| | | actgtccttt cctaataaaa tgaggaaatt gcatcgcatt |
| | | gtctgagtag gtgtcattct attctggggg gtggggtggg |
| | | gcaggacagc aagggggagg attgggaaga caatagcagg |
| | | catgctgggg atgcggtggg ctctatggct tctactgggc |
| | | ggttttatgg acagcaagcg aaccaccggt acccgggccc |
| | | atggcgcgga acccctattt gtttattttt ctaaatacat tcaaatatgt |
| | | atccgctcat gagacaataa ccctgataaa tgcttcaata |
| | | atattgaaaa aggaagagta tgagtattca acatttccgt |
| | | gtcgccctta ttcccttttt tgcggcattt tgccttcctg tttttgctca |
| | | cccagaaacg ctggtgaaag taaaagatgc tgaagatcag |
| | | ttgggtgcac gagtgggtta catcgaactg gatctcaaca |
| | | gcggtaagat ccttgagagt tttcgccccg aagaacgttt |
| | | tccaatgatg agcactttta aagttctgct atgtggcgcg gtattatccc |
| | | gtattgacgc cgggcaagag caactcggtc gccgcataca |
| | | ctattctcag aatgacttgg ttgagtactc accagtcaca |
| | | gaaaagcatc ttacggatgg catgacagta agagaattat |
| | | gcagtgctgc cataaccatg agtgataaca ctgcggccaa |
| | | cttacttctg acaacgatcg gaggaccgaa ggagctaacc |
| | | gcttttttgc acaacatggg ggatcatgta actcgccttg |
| | | atcgttggga accggagctg aatgaagcca taccaaacga |
| | | cgagcgtgac accacgatgc ctgtagcaat ggcaacaacg |
| | | ttgcgcaaac tattaactgg cgaactactt actctagctt |
| | | cccggcaaca attaatagac tggatggagg cggataaagt |
| | | tgcaggacca cttctgcgct cggcccttcc ggctggctgg |
| | | tttattgctg ataaatctgg agccggtgag cgtgggtctc |
| | | gcggtatcat tgcagcactg gggccagatg gtaagccctc |
| | | ccgtatcgta gttatctaca cgacggggag tcaggcaact |
| | | atggatgaac gaaatagaca gatcgctgag ataggtgcct |
| | | cactgattaa gcattggtaa ctgtcagacc aagtttactc atatatactt |
| | | tagattgatt taaaacttca tttttaattt aaaaggatct aggtgaagat |
| | | cctttttgat aatctcatga ccaaaatccc ttaacgtgag ttttcgttcc |
| | | actgagcgtc agaccccgta ctagtactcg agctcgcgaa |
| | | gaaaagatca aaggatcttc ttgagatcct ttttttctgc gcgtaatctg |
| | | ctgcttgcaa acaaaaaaac caccgctacc agcggtggtt |
| | | tgtttgccgg atcaagagct accaactctt tttccgaagg |
| | | taactggctt cagcagagcg cagataccaa atactgttct |
| | | tctagtgtag ccgtagttag gccaccactt caagaactct |
| | | gtagcaccgc ctacatacct cgctctgcta atcctgttac |
| | | cagtggctgc tgccagtggc gataagtcgt gtcttaccgg |
| | | gttggactca agacgatagt taccggataa ggcgcagcgg |
| | | tcgggctgaa cggggggttc gtgcacacag cccagcttgg |
| | | agcgaacgac ctacaccgaa ctgagatacc tacagcgtga |
| | | gctatgagaa agcgccacgc ttcccgaagg gagaaaggcg |
| | | gacaggtatc cggtaagcgg cagggtcgga acaggagagc |
| | | gcacgaggga gcttccaggg ggaaacgcct ggtatcttta |
| | | tagtcctgtc gggtttcgcc acctctgact tgagcgtcga tttttgtgat |
| | | gctcgtcagg ggggcggagc ctatggaaaa acgccagcaa |
| | | cgcggccttt ttacggttcc tggccttttg ctggcctttt gctcacatgt |
| | | tcttgctgct |
| 1 | pSV40-hBAX- | tcgcgatgta cgggccagat ata[MluI] cgcgtt[Begin |
| [functional | BLa [functional | SV40 Promoter]c tgtggaatgt gtgtcagtta gggtgtggaa |
| elements | elements | agtccccagg ctccccagca ggcagaagta tgcaaagcat |
| marked] | marked] | gcatctcaat tagtcagcaa ggaaagtccc caggctcccc |
| | | agcaggcaga agtatgcaaa gcatgcatct caattagtca |
| | | gcaaccatag tcccgcccct aactccgccc atcccgcccc |
| | | taactccgcc cagttccgcc cattctccgc cccatggctg |
| | | actaattttt tttattta[Begin SV40 Replication Origin]tg |
| | | cagaggccga ggccgcct[SfiI]c[Begin Early-Early |
| | | Cap Site 1]g gc[End Early-Early Cap Site |
| | | 1]c[Begin Early-Early Cap Site 2]tctg[End Early- |
| | | Early Cap Site 2]agc[End SV40 Replication |
| | | Origin] tattccagaa gtagtgaaga ggcttttttg |
| | | gaggc[BlnI]ctagg cttttgcaaa aagct[End SV40 |
| | | Promoter][AccIII]ccgga t[ClaI*]cgatcctga |
| | | gaacttcagg [Begin Rabbit Beta-1 Globin |
| | | Intron]gtgagtttgg ggacccttga ttgttctttc tttttcgcta |
| | | ttgtaaaatt catgttatat ggagggggca aagttttcag ggtgttgttt |
| | | agaatgggaa gatgtccctt gtatcaccat ggaccctcat |
| | | gataattttg tttctttcac tttctactct gtt[HindIII and |
| | | HincII]gacaacc attgtctcct cttattttct tttcattttc tgtaactttt |
| | | tcgttaaact ttagcttgca tttgtaacga atttttaaat tcacttttgt |
| | | ttatttgtca gattgtaagt actttctcta atcacttttt tttcaaggca |
| | | atcagggtat attatattgt acttcagcac agttttagag |
| | | aac[MunI]aattgtt ataattaaat gataaggtag aatatttctg |
| | | catataaatt ctggctggcg tggaaatatt cttattggta |
| | | gaaacaacta catcctggtc atcatcctgc ctttctcttt atggttacaa |
| | | tgatatacac tgtttgagat gaggataaaa tactctgagt |
| | | ccaaaccggg cccctctgct aaccatgttc atgccttctt ctttttccta |
| | | cag[End Rabbit Beta-1 Globin Intron][Begin |
| | | Rabit Beta-1 Exon3]ctcctgg gcaacgtgct |
| | | ggttattgt[Begin Exon3 Tagman Probe]g ctgtctcatc |
| | | attttggcaa ag[End Exon3 Tagman Probe]aatt[End |
| | | Rabbit Beta-1 Globin Exon 3]gtaa tacgactcac |
| | | tatagggcga attcgcggtg [Begin hBax]atggacgggt |
| | | ccggggagca gcccagaggc ggggggccca c[Begin |
| | | tmACD5]cagctctga gcagatcatg aagac[End |
| | | tmACD5]agggg cccttttgct tcagggtttc atccaggatc |
| | | gagcagggcg aatggggggg gaggcacccg agctggccct |
| | | ggacccggtg cctcaggatg cgtccaccaa gaagctgagc |
| | | gagtgtctca agcgcatcgg ggacgaactg gacagtaaca |
| | | tggagctgca gaggatgatt gccgccgtgg acacagactc |
| | | cccccgagag gcctttttcc gagtggcag[PvuII]c tgacatgttt |
| | | tctgacggca acttcaactg gggccgggtt gtcgcccttt |
| | | tctactttgc cagcaaactg gtgctcaagg ccctgtgcac |
| | | caaggtgccg gaactgatca gaaccatcat gggctggaca |
| | | ttggacttcc tccgggagcg gctgttgggc |
| | | tg[BamHI]gatccaag accagggtgg ttgggacggc |
| | | ctcctctcct actttgggac gcccacgtgg cagaccgtga |
| | | ccatctttgt ggcgggagtg ctcaccgcct cactcaccat |
| | | ctggaagaag atgggct[End hBax]gag aattctgcag |
| | | at[EcoRV]atccagca cagtggc[NotI]ggc |
| | | cgctcgagtc[XbaI] tagagggccc gtttaaaccc gctgatcagc |
| | | ctcga[Begin Bovine Growth Hormone |
| | | Polyadenylation Signal]ctgtg ccttctagtt gccagccatc |
| | | tgttgtttgc ccctcccccg tgccttcctt gaccctggaa |
| | | ggtgccactc ccactgtcct ttcctaataa aatgaggaaa |
| | | ttgcatcgca ttgtctgagt aggtgtcatt ctattctggg |
| | | gggtggggtg gggcaggaca gcaaggggga ggattgggaa |
| | | gacaatagca ggcatgctgg ggatgcggtg ggctctatgg[End |
| | | Bovine Growth Hormone Polyadenylation |
| | | Signal] cttctactgg gcggttttat ggacagcaag |
| | | cgaacca[AgeI][Begin RS1]ccg[Acc65I] gtac[XmaI |
| | | and KpmI]cc[SmaI]gggc ccatgg[End RS1][Begin |
| | | BLa Promoter]cgcg gaacccctat ttgtttattt ttctaaatac |
| | | attcaaatat gtatccgctc aaccctgata aatgcttcaa taatattgaa |
| | | aaaggaagag t[End BLa Promoter][Begin Beta- |
| | | lactamase]atgagtatt caacatttcc gtgtcgccct tattcccttt |
| | | tttgcggcat tttgccttcc tgtttttgct cacccagaaa cgctggtgaa |
| | | agtaaaagat gctgaagatc agttgggtgc acgagtgggt |
| | | tacatcgaac tggatctcaa cagcggtaag atccttgaga |
| | | gttttcgccc cgaagaacgt tttccaatga tgagcacttt taaagttctg |
| | | ctatgtggcg cggtattatc ccgtattgac gccgggcaag |
| | | agcaactcgg tcgccgcata cactattctc agaatgactt |
| | | ggttgagtac tcaccagtca cagaaaagca tcttacggat |
| | | ggcatgacag taagagaatt atgcagtgct gccataacca |
| | | tgagtgataa cactgcggcc aacttacttc tgacaacgat[PvuI] |
| | | cggaggaccg aaggagctaa ccgctttttt gcacaacatg |
| | | ggggatcatg taactcgcct tgatcgttgg gaaccggagc |
| | | tgaatgaagc cataccaaac gacgagcgtg acaccacgat |
| | | gcctgtagca atggcaacaa cgttgcgcaa actattaact |
| | | ggcgaactac ttactctagc ttcccggcaa caat[VspI]taatag |
| | | actggatgga ggcggataaa gttgcaggac cacttctgcg |
| | | ctcggccctt ccggctggct ggtttattgc tgataaatct |
| | | ggagccggtg agcgtgggtc tcgcggtatc attgcagcac |
| | | tggggccaga tggtaagccc tcccgtatcg tagttatcta |
| | | cacgacgggg agtcaggcaa ctatggatga acgaaataga |
| | | cagatcgctg agataggtgc ctcactgatt aagcattggt aa[End |
| | | Beta-lactamase][Begin BLa Transcription |
| | | Terminator]ctgtcaga ccaagtttac tcatatatac tttagattga |
| | | tttaaaactt catttttaat ttaaaaggat ctaggtgaag atcctttttg |
| | | ataatctcat gaccaaaatc ccttaacgtg agttttcgtt |
| | | ccactgagcg tcagaccccg t[End BLa Transcription |
| | | Terminator][Begin RS2]a[SpeI]ctagtact |
| | | cgag[Eco53KI]ct[SacI]cgcg a[End RS2][Begin |
| | | ColE1 Ori]agaaaagat caaaggatct tcttgagatc ctttttttct |
| | | gcgcgtaatc tgctgcttgc aaacaaaaaa accaccgcta |
| | | ccagcggtgg tttgtttgcc ggatcaagag ctaccaactc |
| | | tttttccgaa ggtaactggc ttcagcagag cgcagatacc |
| | | aaatactgtt cttctagtgt agccgtagtt aggccaccac |
| | | ttcaagaact ctgtagcacc gcctacatac ctcgctctgc |
| | | taatcctgtt accagtggct gctgccagtg gcgataagtc |
| | | gtgtcttacc gggttggact caagacgata gttaccggat |
| | | aaggcgcagc ggtcgggctg aacggggggt tgtgcacac |
| | | agcccagctt ggagcgaacg acctacaccg aactgagata |
| | | cctacagcgt gagctatgag aaagcgc[HaeII]cac |
| | | gcttcccgaa gggagaaagg cggacaggta tccggtaagc |
| | | ggcagggtcg gaacaggaga gcgcacgagg gagcttccag |
| | | ggggaaacgc ctggtatctt tatagtcctg tcgggtttcg |
| | | ccacctctga cttgagcgtc gatttttgtg atgctcgtca |
| | | ggggggcgga gcctatggaa aaacgcc[End ColE1 |
| | | Ori]agc aacgcggcct ttttacggtt cctggccttt tgctggcctt |
| | | ttgctcacat gttcttgctg ct |
| 2 | pSV40-sGAD55- | tcgcgatgta cgggccagat atacgcgtt[SV40 Promoter]c |
| [functional | BLa | tgtggaatgt gtgtcagtta gggtgtggaa agtccccagg |
| elements | [functional | ctccccagca ggcagaagta tgcaaagcat gcatctcaat |
| marked] | elements | tagtcagcaa ggaaagtccc caggctcccc agcaggcaga |
| | marked] | agtatgcaaa gcatgcatct caattagtca gcaaccatag |
| | | tcc[Begin ADS3700 Pyrosequence Target]cgcccct |
| | | aactccgccc atcccgcccc taactccgcc cagttccgcc |
| | | cattctccgc cccatggctg actaattttt tttattt[End ADS3700 |
| | | Pyrosequence Target]a[Begin SV40 Replication |
| | | Origin]tg cagaggccga ggccgcct[SfII]cg |
| | | gcctctgagc[End SV40 Replication Origin] |
| | | tattccagaa gtagtgaaga ggcttttttg gaggc[BlnI]ctagg |
| | | cttttgcaaa aagct[AccIII][End SV40 |
| | | Promoter]ccgga t[ClaI]cgatcctga gaacttcagg |
| | | [Rabbit Beta-1 Globin Intron]gtgagtttgg |
| | | ggacccttga ttgttctttc tttttcgcta ttgtaaaatt catgttatat |
| | | ggagggggca aagttttcag ggtgttgttt agaatgggaa |
| | | gatgtccctt gtatcaccat ggaccctcat gataattttg tttctttcac |
| | | tttctactct gtt[HindIII and HincII]gacaacc attgtctcct |
| | | cttattttct tttcattttc tgtaactttt tcgttaaact ttagcttgca |
| | | tttgtaacga atttttaaat tcacttttgt ttatttgtca gattgtaagt |
| | | actttctcta atcacttttt tttcaaggca atcagggtat attatattgt |
| | | acttcagcac agttttagag aac[MunI]aattgtt ataattaaat |
| | | gataaggtag aatatttctg catataaatt ctggctggcg tggaaatatt |
| | | cttattggta gaaacaacta catcctggtc atcatcctgc ctttctcttt |
| | | atggttacaa tgatatacac tgtttgagat gaggataaaa |
| | | tactctgagt ccaaaccggg cccctctgct aaccatgttc |
| | | atgccttctt ctttttccta cag[End Rabbit Beta-1 Globin |
| | | Intron][Begin Rabbit Beta-1 Globin Exon |
| | | 3]ctcctgg gcaacgtgct ggttatt[Begin Exon 3 Tagman |
| | | Probe]gtg ctgtctcatc attttggcaa ag[End Exon 3 |
| | | Tagman Probe]aatt[End Rabbit Beta-1 Globin |
| | | 3]gtaa tacgactcac tatagggcga attcg[BamHI]gatcc |
| | | actagtccag tgtggtggaa ttctgcagat atccagcaca |
| | | gtggc[NotI]ggccg ctcgacggta t[ClaI]cgataagct |
| | | tgatatcgaa ttcgttggga ttttctaga[Begin sGAD55]a |
| | | tgtacaggat gcaactcctg tcttgcattg cactaagtct tgcacttgtc |
| | | acaaacagtg cacctactta cgcgtttctc catgcaacag |
| | | acctgctgcc[NaeI] ggcgtgtgat ggagaaaggc ccactttggc |
| | | gtttctgcaa gatgttatga acattttact tcagtatgtg gtgaaaagtt |
| | | tcgatagatc aaccaaagtg attgatttcc attatcctaa tgagcttctc |
| | | caagaatata attgggaatt ggcagaccaa ccacaaaatt |
| | | tggaggaaat tttgatgcat tgccaaacaa ctctaaaata |
| | | tgcaattaaa acagggcatc ctagatactt caatcaactt tctactggtt |
| | | tggatatggt tggattagca gcagactggc tgacatcaac |
| | | agcaaatact aacatgttca cctatgaaat tgctccagta tttgtgcttt |
| | | tggaatatgt cacactaaag aaaatgagag aaatcattgg |
| | | ctgg[BalI]ccaggg ggctctg[Begin ADS3701 |
| | | Pyrosequence Target]gcg atgggatatt ttctcccggt |
| | | gg[NarI]cgccatat ctaacatgta tgccatgatg atcgcacgct |
| | | ttaagatgtt cccagaag[End ADS3701 Pyrosequence |
| | | Target]tc aaggagaaag gaatggctgc tcttcccagg |
| | | ctcattgcct tcacgtctga acatagtcat ttttctctca agaagggagc |
| | | tgcagcctta gggattggaa cagacagcgt gattctgatt |
| | | aaatgtgatg agagagggaa aatgattcca tctgatcttg |
| | | aaagaaggat tcttgaagcc aaacagaaag ggtttgttcc |
| | | tttcctcgtg agtgccacag[PvuII] ctggaaccac cgtgtacgga |
| | | gcatttgacc ccctcttagc tgtcgctgac atttgcaaaa |
| | | agtataa[BglII]gat ctggatgcat gtggatgcag cttggggtgg |
| | | gggattactg atgtcccgaa aacacaagtg gaaactgagt |
| | | ggcgtggaga gggccaactc tgtgacgtgg aatccacaca |
| | | agatgatggg agtccctttg cagtgctctg ctctcctggt |
| | | tagagaagag ggattgatgc agaattgcaa ccaaatgcat |
| | | gcctcctacc tctttcagca agataaacat tatgacctgt |
| | | cctatgacac tggagacaag gccttacagt gcggacgcca |
| | | cgttgatgtt tttaaactat ggctgatgtg gagggcaaag |
| | | gggactaccg ggtttgaagc gcatgttgat aaatgtttgg |
| | | agttggcaga gtatttatac aaaaccgaga aggatatgag |
| | | atggtgtttg atgggaagcc[Bpu10I] tcagcacaca |
| | | aatgtctgct tctggtacat tcctccaagc ttgcgtactc |
| | | tggaagacaa tgaagagaga atgagtcgcc tctcgaaggt |
| | | ggctccagtg attaaagcca gaatgatgga gtatggaacc |
| | | acaatggtca gctaccaacc cttgggagac |
| | | a[Tth111I]aggtcaatt tcgtccgcat ggtcatctca |
| | | aacccagcgg caactcacca agacattgac ttcctgattg |
| | | aagaaataga acgccttgga caagatttat aa[End |
| | | sGAD55]taaccttg ctcaccaagc tgttcacttc ttcgagtcta |
| | | gagggcccgt ttaaacccgc t[BclI]gatcagcct cga[Begin |
| | | Bovine Growth Hormone Polyadenylation |
| | | Signal]ctgtgcc ttctagttgc cagccatctg ttgtttgccc |
| | | ctcccccgtg ccttccttga ccctggaagg tgccactccc |
| | | actgtccttt cctaataaaa tgaggaaatt gcatcgcatt |
| | | gtctgagtag gtgtcattct attctggggg gtggggtggg |
| | | gcaggacagc aagggggagg attgggaaga caatagcagg |
| | | catgctgggg atg[Begin ADS3702 Partial |
| | | Pyrosequence Target]cggtggg ctctatgg[End |
| | | Bovine Growth Hormone Polyadenylation |
| | | Signal]ct tctactgggc ggttttatgg acagcaagcg |
| | | aacc[Begin RS1]a[AgeI]ccg[Acc65I]gt ac[XmaI |
| | | and KphI]ccggg[SmaI]ccc atggEnd RS1][Begin |
| | | BLa Promoter]cgcgga acccctattt gtttattttt ctaa[End |
| | | ADS3702 Partial Pyrosequencing Target]atacat |
| | | tcaaatatgt atccgctcat gagacaataa ccctgataaa |
| | | tgcttcaata atattgaaaa aggaagagt[Eng BLa |
| | | Promoter][Begin Beta-lactamase]a tgagtattca |
| | | acatttccgt gtcgccctta ttcccttttt tgcggcattt tgccttcctg |
| | | tttttgctca cccagaaacg ctggtgaaag taaaagatgc |
| | | tgaagatcag ttgggtgcac gagtgggtta catcgaactg |
| | | gatctcaaca gcggtaagat ccttgagagt tttcgccccg |
| | | aagaacgttt tccaatgatg agcactttta aagttctgct |
| | | atgtggcgcg gtattatccc gtattgacgc cgggcaagag |
| | | caactcggtc gccgcataca ctattctcag aatgacttgg |
| | | ttgagtactc accagtcaca gaaaagcatc ttacggatgg |
| | | catgacagta agagaattat gcagtgctgc cataaccatg |
| | | agtgataaca ctgcggccaa cttacttctg acaacgat[PvuI]cg |
| | | gaggaccgaa ggagctaacc gcttttttgc acaacatggg |
| | | ggatcatgta actcgccttg atcgttggga accggagctg |
| | | aatgaagcca taccaaacga cgagcgtgac accacgatgc |
| | | ctgtagcaat ggcaacaacg ttgcgcaaac tattaactgg |
| | | cgaactactt actctagctt cccggcaaca at[VspI]taatagac |
| | | tggatggagg cggataaagt tgcaggacca cttctgcgct |
| | | cggcccttcc ggctggctgg tttattgctg ataaatctgg |
| | | agccggtgag cgtgggtctc gcggtatcat tgcagcactg |
| | | gggccagatg gtaagccctc ccgtatcgta gttatctaca |
| | | cgacggggag tcaggcaact atggatgaac gaaatagaca |
| | | gatcgctgag ataggtgcct cactgattaa gcattggta[End |
| | | Beta-lactamase] [Begin BLa Transcription |
| | | Terminatorla ctgtcagacc aagtttactc atatatactt |
| | | tagattgatt taaaacttca tttttaattt aaaaggatct aggtgaagat |
| | | cctttttgat aatctcatga ccaaaatccc ttaacgtgag ttttcgttcc |
| | | actgagcgtc agaccccgt[End BLa Transcription |
| | | Terminator][End RS2]a ctagtac[XhoI]tcg |
| | | ag[EcoICRI]ct[SacI]cgcga[End RS2][Begin |
| | | ColE1 Ori]a gaaaagatca aaggatcttc ttgagatcct |
| | | ttttttctgc gcgtaatctg ctgcttgcaa acaaaaaaac |
| | | caccgctacc agcggtggtt tgtttgccgg atcaagagct |
| | | accaactctt tttccgaagg taactggctt cagcagagcg |
| | | cagataccaa atactgttct tctagtgtag ccgtagttag |
| | | gccaccactt caagaactct gtagcaccgc ctacatacct |
| | | cgctctgcta atcctgttac cagtggctgc tgccagtggc |
| | | gataagtcgt gtcttaccgg gttggactca agacgatagt |
| | | taccggataa ggcgcagcgg tcgggctgaa cggggggttc |
| | | gtgcacacag cccagcttgg agcgaacgac ctacaccgaa |
| | | ctgagatacc tacagcgtga gctatgagaa agcgccacgc |
| | | ttcccgaagg gagaaaggcg gacaggtatc cggtaagcgg |
| | | cagggtcgga acaggagagc gcacgaggga gcttccaggg |
| | | ggaaacgcct ggtatcttta tagtcctgtc gggtttcgcc |
| | | acctctgact tgagcgtcga tttttgtgat gctcgtcagg |
| | | ggggcggagc ctatggaaaa acgcc[End ColE1 |
| | | Ori]agcaa cgcggccttt ttacggttcc tggccttttg ctggcctttt |
| | | gctcacatgt tcttgctgct |
| 3 | pSV40- | tcgcgatgta cgggccagat atacgcgttc tgtggaatgt |
| | sGAD55 + hBAX- | gtgtcagtta gggtgtggaa agtccccagg ctccccagca |
| | BLa | ggcagaagta tgcaaagcat gcatctcaat tagtcagcaa |
| | | ggaaagtccc caggctcccc agcaggcaga agtatgcaaa |
| | | gcatgcatct caattagtca gcaaccatag tcccgcccct |
| | | aactccgccc atcccgcccc taactccgcc cagttccgcc |
| | | cattctccgc cccatggctg actaattttt tttatttatg cagaggccga |
| | | ggccgcctcg gcctctgagc tattccagaa gtagtgaaga |
| | | ggcttttttg gaggcctagg cttttgcaaa aagctccgga |
| | | tcgatcctga gaacttcagg gtgagtttgg ggacccttga ttgttctttc |
| | | tttttcgcta ttgtaaaatt catgttatat ggagggggca aagttttcag |
| | | ggtgttgttt agaatgggaa gatgtccctt gtatcaccat |
| | | ggaccctcat gataattttg tttctttcac tttctactct gttgacaacc |
| | | attgtctcct cttattttct tttcattttc tgtaactttt tcgttaaact |
| | | ttagcttgca tttgtaacga atttttaaat tcacttttgt ttatttgtca |
| | | gattgtaagt actttctcta atcacttttt tttcaaggca atcagggtat |
| | | attatattgt acttcagcac agttttagag aacaattgtt ataattaaat |
| | | gataaggtag aatatttctg catataaatt ctggctggcg tggaaatatt |
| | | cttattggta gaaacaacta catcctggtc atcatcctgc ctttctcttt |
| | | atggttacaa tgatatacac tgtttgagat gaggataaaa |
| | | tactctgagt ccaaaccggg cccctctgct aaccatgttc |
| | | atgccttctt ctttttccta cagctcctgg gcaacgtgct ggttattgtg |
| | | ctgtctcatc attttggcaa agaattgtaa tacgactcac |
| | | tatagggcga attcggatcc actagtccag tgtggtggaa |
| | | ttctgcagat atccagcaca gtggcggccg ctcgacggta |
| | | tcgataagct tgatatcgaa ttcgttggga ttttctagaa tgtacaggat |
| | | gcaactcctg tcttgcattg cactaagtct tgcacttgtc cacctactta |
| | | cgcgtttctc catgcaacag acctgctgcc ggcgtgtgat |
| | | ggagaaaggc ccactttggc gtttctgcaa gatgttatga |
| | | acattttact tcagtatgtg gtgaaaagtt tcgatagatc |
| | | aaccaaagtg attgatttcc attatcctaa tgagcttctc caagaatata |
| | | attgggaatt ggcagaccaa ccacaaaatt tggaggaaat |
| | | tttgatgcat tgccaaacaa ctctaaaata tgcaattaaa |
| | | acagggcatc ctagatactt caatcaactt tctactggtt tggatatggt |
| | | tggattagca gcagactggc tgacatcaac agcaaatact |
| | | aacatgttca cctatgaaat tgctccagta tttgtgcttt tggaatatgt |
| | | cacactaaag aaaatgagag aaatcattgg ctggccaggg |
| | | ggctctggcg atgggatatt ttctcccggt ggcgccatat |
| | | ctaacatgta tgccatgatg atcgcacgct ttaagatgtt |
| | | cccagaagtc aaggagaaag gaatggctgc tcttcccagg |
| | | ctcattgcct tcacgtctga acatagtcat ttttctctca agaagggagc |
| | | tgcagcctta gggattggaa cagacagcgt gattctgatt |
| | | aaatgtgatg agagagggaa aatgattcca tctgatcttg |
| | | aaagaaggat tcttgaagcc aaacagaaag ggtttgttcc |
| | | tttcctcgtg agtgccacag ctggaaccac cgtgtacgga |
| | | gcatttgacc ccctcttagc tgtcgctgac atttgcaaaa |
| | | agtataagat ctggatgcat gtggatgcag cttggggtgg |
| | | gggattactg atgtcccgaa aacacaagtg gaaactgagt |
| | | ggcgtggaga gggccaactc tgtgacgtgg aatccacaca |
| | | agatgatggg agtccctttg cagtgctctg ctctcctggt |
| | | tagagaagag ggattgatgc agaattgcaa ccaaatgcat |
| | | gcctcctacc tctttcagca agataaacat tatgacctgt |
| | | cctatgacac tggagacaag gccttacagt gcggacgcca |
| | | cgttgatgtt tttaaactat ggctgatgtg gagggcaaag |
| | | gggactaccg ggtttgaagc gcatgttgat aaatgtttgg |
| | | agttggcaga gtatttatac aacatcataa aaaaccgaga |
| | | aggatatgag atggtgtttg atgggaagcc tcagcacaca |
| | | aatgtctgct tctggtacat tcctccaagc ttgcgtactc |
| | | tggaagacaa tgaagagaga atgagtcgcc tctcgaaggt |
| | | ggctccagtg attaaagcca gaatgatgga gtatggaacc |
| | | acaatggtca gctaccaacc cttgggagac aaggtcaatt |
| | | tcgtccgcat ggtcatctca aacccagcgg caactcacca |
| | | agacattgac ttcctgattg aagaaataga acgccttgga |
| | | caagatttat aataaccttg ctcaccaagc tgttcacttc tagaatcact |
| | | agtgcggccg cctgcaggtc gaggatccag aggtaccgag |
| | | ctcgaattct gcagatatcc atcacactgg cggccgcctg |
| | | caggtcgagg atccagaggt accgagctcg aattctgcag |
| | | atatccatca cactggcggc cgcctgcagg tcgaggatcc |
| | | agaggtaccg agctcgaatt ctgcagatat ccatcacact |
| | | ggcggccgcc tgcaggtcga ggatccagag gtaccgagct |
| | | cgaattctgc agatatccat cacactggcg gccgctctag |
| | | aactagtgga tcccccgggc tgcaggaatt cgataaacct |
| | | agaaacacga ctcactatag ggcgaattcc gccccccccc |
| | | ctaacgttac tggccgaagc cgcttggaat aaggccggtg |
| | | tgcgtttgtc tatatgttat tttccaccat attgccgtct tttggcaatg |
| | | tgagggcccg gaaacctggc cctgtcttct tgacgagcat |
| | | tcctaggggt ctttcccctc tcgccaaagg aatgcaaggt |
| | | ctgttgaatg tcgtgaagga agcagttcct ctggaagctt |
| | | cttgaagaca aacaacgtct gtagcgaccc tttgcaggca |
| | | gcggaacccc ccacctggcg acaggtgcct ctgcggccaa |
| | | aagccacgtg tataagatac acctgcaaag gcggcacaac |
| | | cccagtgcca cgttgtgagt tggatagttg tggaaagagt |
| | | caaatggctc tcctcaagcg tattcaacaa ggggctgaag |
| | | gatgcccaga aggtacccca ttgtatggga tctgatctgg |
| | | ggcctcggtg cacatgcttt acatgtgttt agtcgaggtt |
| | | aaaaaaacgt ctaggccccc cgaaccacgg ggacgtggtt |
| | | ttcctttgaa aaacacgatt attatattgc ctctaggatg |
| | | gacgggtccg gggagcagcc cagaggcggg gggcccacca |
| | | gctctgagca gatcatgaag acaggggccc ttttgcttca |
| | | gggtttcatc caggatcgag cagggcgaat ggggggggag |
| | | gcacccgagc tggccctgga cccggtgcct caggatgcgt |
| | | ccaccaagaa gctgagcgag tgtctcaagc gcatcgggga |
| | | cgaactggac agtaacatgg agctgcagag gatgattgcc |
| | | gccgtggaca cagactcccc ccgagaggcc tttttccgag |
| | | tggcagctga catgttttct gacggcaact tcaactgggg |
| | | ccgggttgtc gcccttttct actttgccag caaactggtg |
| | | ctcaaggccc tgtgcaccaa ggtgccggaa ctgatcagaa |
| | | ccatcatggg ctggacattg gacttcctcc gggagcggct |
| | | gttgggctgg atccaagacc agggtggttg ggacggcctc |
| | | ctctcctact ttgggacgcc cacgtggcag accgtgacca |
| | | tctttgtggc gggagtgctc accgcctcac tcaccatctg |
| | | gaagaagatg ggctgagaat tctgcagata tatcaagctt |
| | | atcgataccg tcgagtctag agggcccgtt taaacccgct |
| | | gatcagcctc gactgtgcct tctagttgcc agccatctgt |
| | | tgtttgcccc tcccccgtgc cttccttgac cctggaaggt |
| | | gccactccca ctgtcctttc ctaataaaat gaggaaattg |
| | | catcgcattg tctgagtagg tgtcattcta ttctgggggg |
| | | tggggtgggg caggacagca agggggagga ttgggaagac |
| | | aatagcaggc atgctgggga tgcggtgggc tctatggctt |
| | | ctactgggcg gttttatgga cagcaagcga accaccggta |
| | | cccgggccca tggcgcggaa cccctatttg tttatttttc taaatacatt |
| | | caaatatgta tccgctcatg agacaataac cctgataaat |
| | | gcttcaataa tattgaaaaa ggaagagtat gagtattcaa |
| | | catttccgtg tcgcccttat tccctttttt gcggcatttt gccttcctgt |
| | | ttttgctcac ccagaaacgc tggtgaaagt aaaagatgct |
| | | gaagatcagt tgggtgcacg agtgggttac atcgaactgg |
| | | atctcaacag cggtaagatc cttgagagtt ttcgccccga |
| | | agaacgtttt ccaatgatga gcacttttaa agttctgcta |
| | | tgtggcgcgg tattatcccg tattgacgcc gggcaagagc |
| | | aactcggtcg ccgcatacac tattctcaga atgacttggt |
| | | tgagtactca ccagtcacag aaaagcatct tacggatggc |
| | | atgacagtaa gagaattatg cagtgctgcc ataaccatga |
| | | gtgataacac tgcggccaac ttacttctga caacgatcgg |
| | | aggaccgaag gagctaaccg cttttttgca caacatgggg |
| | | gatcatgtaa ctcgccttga tcgttgggaa ccggagctga |
| | | atgaagccat accaaacgac gagcgtgaca ccacgatgcc |
| | | tgtagcaatg gcaacaacgt tgcgcaaact attaactggc |
| | | gaactactta ctctagcttc ccggcaacaa ttaatagact |
| | | ggatggaggc ggataaagtt gcaggaccac ttctgcgctc |
| | | ggcccttccg gctggctggt ttattgctga taaatctgga |
| | | gccggtgagc gtgggtctcg cggtatcatt gcagcactgg |
| | | ggccagatgg taagccctcc cgtatcgtag ttatctacac |
| | | gacggggagt caggcaacta tggatgaacg aaatagacag |
| | | atcgctgaga taggtgcctc actgattaag cattggtaac |
| | | tgtcagacca agtttactca tatatacttt agattgattt aaaacttcat |
| | | ttttaattta aaaggatcta ggtgaagatc ctttttgata atctcatgac |
| | | caaaatccct taacgtgagt tttcgttcca ctgagcgtca |
| | | gaccccgtac tagtactcga gctcgcgaag aaaagatcaa |
| | | aggatcttct tgagatcctt tttttctgcg cgtaatctgc tgcttgcaaa |
| | | caaaaaaacc accgctacca gcggtggttt gtttgccgga |
| | | tcaagagcta ccaactcttt ttccgaaggt aactggcttc |
| | | agcagagcgc agataccaaa tactgttctt ctagtgtagc |
| | | cgtagttagg ccaccacttc aagaactctg tagcaccgcc |
| | | tacatacctc gctctgctaa tcctgttacc agtggctgct |
| | | gccagtggcg ataagtcgtg tcttaccggg ttggactcaa |
| | | gacgatagtt accggataag gcgcagcggt cgggctgaac |
| | | ggggggttcg tgcacacagc ccagcttgga gcgaacgacc |
| | | tacaccgaac tgagatacct acagcgtgag ctatgagaaa |
| | | gcgccacgct tcccgaaggg agaaaggcgg acaggtatcc |
| | | ggtaagcggc agggtcggaa caggagagcg cacgagggag |
| | | cttccagggg gaaacgcctg gtatctttat agtcctgtcg |
| | | ggtttcgcca cctctgactt gagcgtcgat ttttgtgatg |
| | | ctcgtcaggg gggcggagcc tatggaaaaa cgccagcaac |
| | | gcggcctttt tacggttcct ggccttttgc tggccttttg ctcacatgtt |
| | | cttgctgct |

Functional elements in SEQ ID NO.: 3 are similar to those in SEQ ID NOs: 1 and 2.

### EXAMPLES

### Example 1: Method of Enzymatic Methylation

1.25 μg of pSV40-sGAD55 source DNA, in either linear or plasmid form (plasmid form depicted in FIG. 2A and FIG. 2B), 160 μM SAM, 50 mM potassium acetate, 20 mM Tris acetate, 10 mM magnesium acetate, and 100 μg/mL BSA at were combined at 25° C. to form a partial reaction solution with pH 7.9. Various amounts of Spiroplasma derived M.SssI methyltransferase were added to complete the reaction solutions, which were then incubated at a selected temperature for a selected time. The reaction was then quenched at 65° C. for 20 minutes.

The fractional methylation level and methylation patterns were assessed by digesting the enzymatically methylated DNA with HpaII and KpnI (if not previously linearized using KpnI), followed by agarose gel electrophoresis of the digested DNA as shown in FIG. 6.

HpaII, if allowed to fully digest pSV40-sGAD55, produces sixteen DNA fragments of the following sizes: 698 bp, 672 bp, 586 bp, 550 bp, 427 bp, 424 bp, 367 bp, 333 bp, 242 bp, 190 bp, 147 bp, 110 bp, 67 bp, 34 bp, 26 bp, 7 bp.

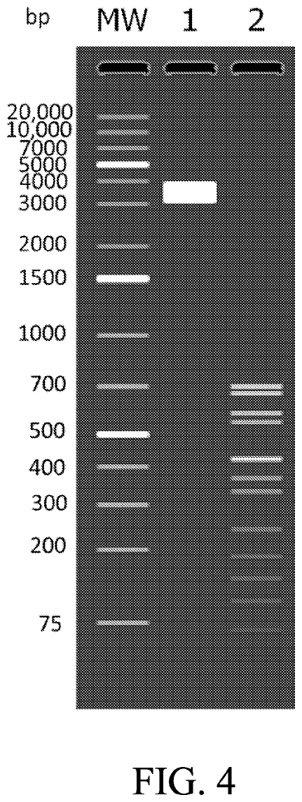

FIG. 4 provides an example diagram of what methylated pSV40-sGAD55 source DNA that has a nearly 100% fractional methylation level (e.g. every CpG site is methylated) may look like on an agarose gel after HpaII digestion (lane 1), as well as an example diagram of what an entirely unmethylated pSV40-sGAD55 source DNA may look like on an agarose gel after HpaII digestion (lane 2).

FIG. 5 shows the results of reaction samples with plasmid pSV40-sGAD55 source DNA after incubation at 37° C. with 20U M.SssI for one hour. The inability of HpaII to digest methylated plasmid, while unmethylated plasmid was digested, indicates that M.SssI was able to methylate the source DNA to a nearly 100% fractional methylation level under these conditions. Both methylated and unmethylated plasmid was digested by MspI, which is not sensitive to methylation.

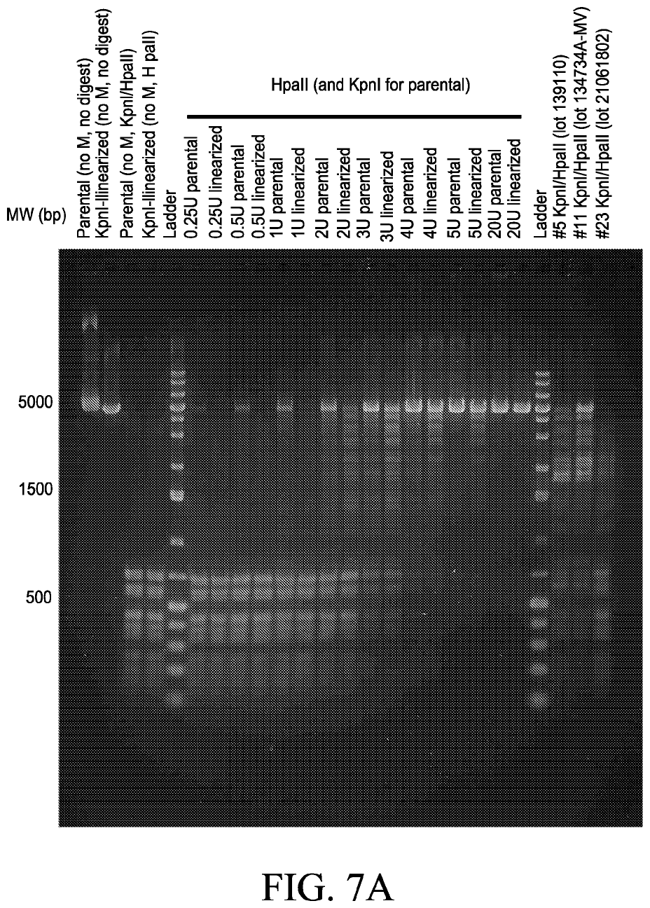

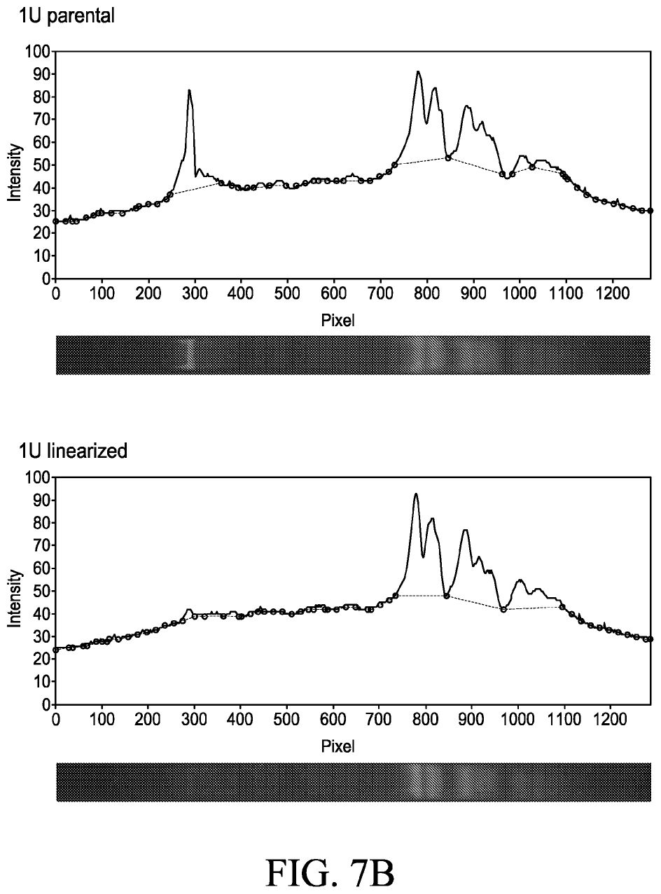

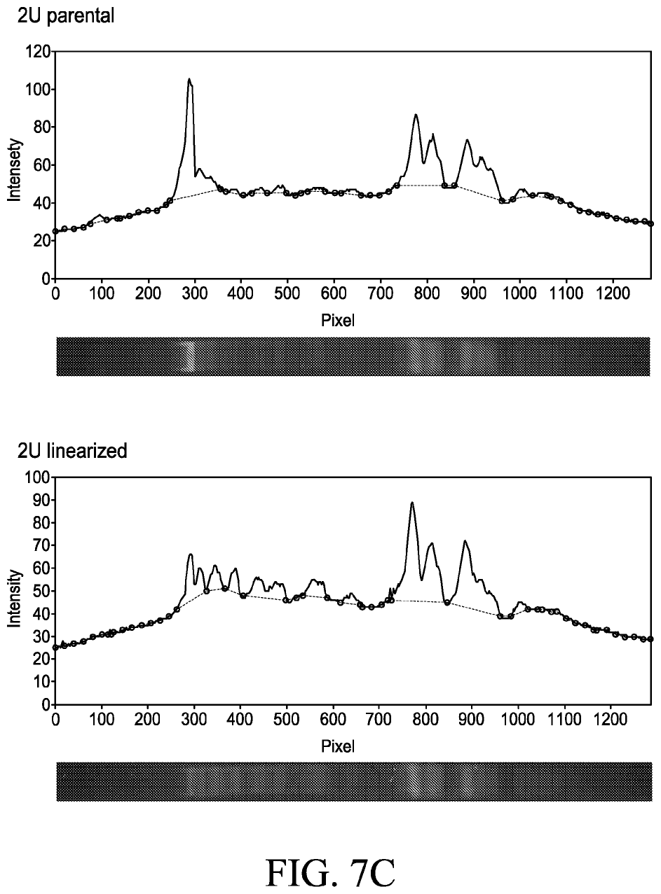

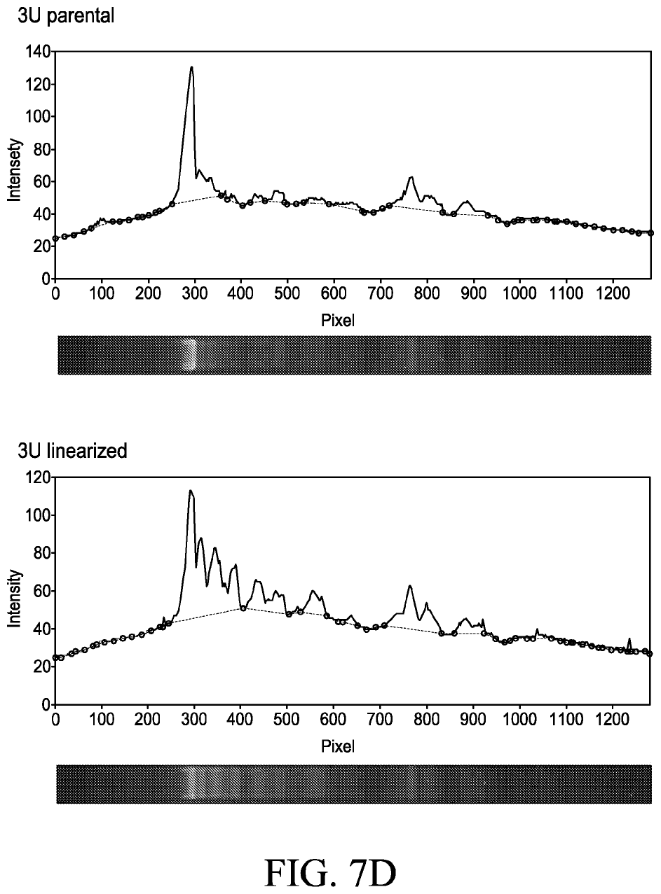

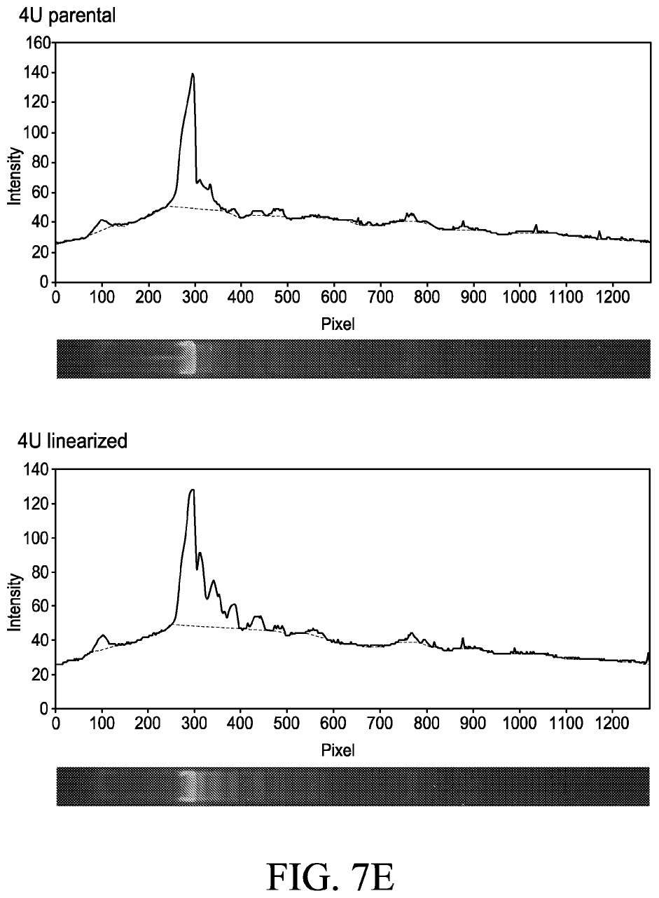

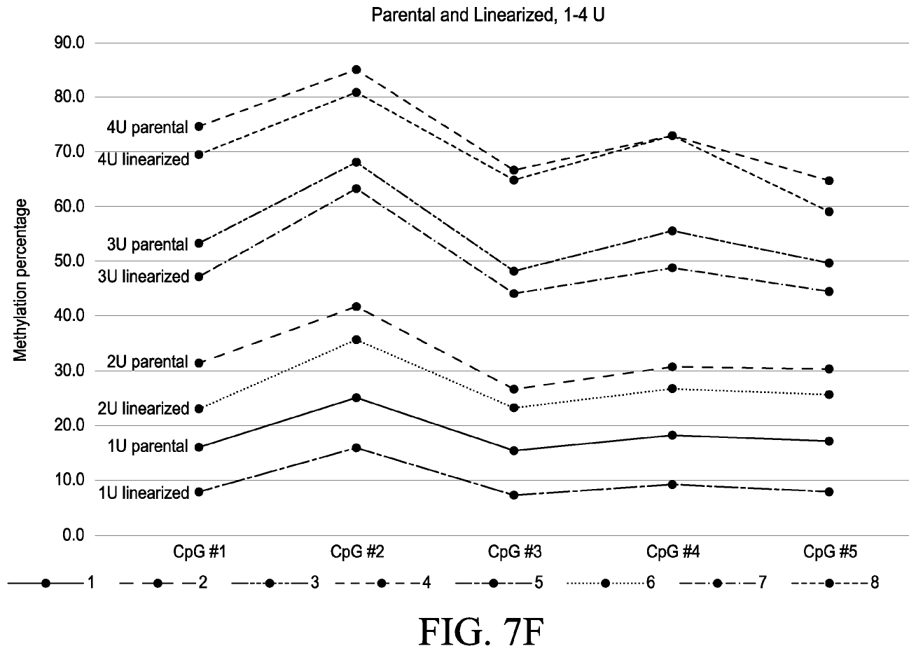

FIG. 7A shows the results for reaction samples with plasmid (parental) source pSV40-sGAD55 DNA or pSV40-sGAD55 DNA linearized using KpnI after incubation at 34° C. with various amounts of M.SssI methyltransferase for 60 minutes, as shown in FIG. 7A. Samples #5, #11, and #23 are bacterially methylated pSV40-sGAD55 DNA that were also digested using HpaII/KpnI in the same manner as the enzymatically methylated samples. The absence of bands and presence only of about 5000 bp DNA, as seen in the “no digest” control samples and in reaction samples with 20U M.SssI methyltransferase, indicates no cleavage by HpaII. In enzymatically methylated samples, such as the 20 U M.SssI methyltransferase, this indicates a fractional methylation level of nearly 100%. Conversely, the presence of only small DNA fragments of about 600 bp or less, as seen in the “no methylation” controls indicates complete digestion with HpaII. Intermediate bands of sizes in a range from about 600 bp to about 4000 bp indicate that the source DNA was methylated at some CpG sites, but not at others. The presence and relative intensity of bands of particular sizes may be used to determine fractional methylation level. For example, if one CpG site is substantially not methylated in the DNA sample, then a band size requiring cleavage at that site will be present and will be relatively intense as compared to bands requiring cleavage at a different site. Relative intensity of each size band may also be used to estimate the CpG site specific methylation level of sites that require methylation in order to produce or not produce a band of a particular size. If CpG site specific methylation levels are determined for all CpG methylation sites in the DNA sample, then the mean whole DNA methylation level may also be calculated.

Pyrosequencing was performed on the plasmid (parental) and linear source DNA. Results are presented in FIGS. 7B, 7C, 7D, 7E, and 7F and in Table 2. Five potential CpG methylation sites were examined and the CpG site specific methylation level of each site was determined.

TABLE 2
Pyrosequencing Results For Various Amounts of
Methylation Enzyme (In Vitro Methylation)

| Sample | Methylation Sites | | | | | Overall Region | | | |
|---|---|---|---|---|---|---|---|---|---|
| ID | CpG#1 | Cpg#2 | CpG#3 | CpG#4 | CpG#5 | Mean | St. Dev. | Min. | Max. |
| Parental, | 16.1 | 25.1 | 15.4 | 18.3 | 17.3 | 18.4 | 3.9 | 15.4 | 25.1 |
| 1 U | | | | | | | | | |
| Linearized, | 7.9 | 15.9 | 7.3 | 9.4 | 7.9 | 9.7 | 3.6 | 7.3 | 15.9 |
| 1 U | | | | | | | | | |
| Parental, | 31.4 | 41.7 | 26.7 | 30.7 | 30.3 | 32.2 | 5.6 | 26.7 | 41.7 |
| 2 U | | | | | | | | | |
| Linearized, | 23.0 | 35.7 | 23.3 | 26.8 | 25.7 | 26.9 | 5.2 | 23.0 | 35.7 |
| 2 U | | | | | | | | | |
| Parental, | 53.3 | 68.1 | 48.1 | 55.6 | 49.7 | 55.0 | 7.9 | 48.1 | 68.1 |
| 3 U | | | | | | | | | |
| Linearized, | 47.3 | 63.3 | 44.1 | 48.8 | 44.5 | 49.6 | 7.9 | 44.1 | 63.3 |
| 3 U | | | | | | | | | |
| Parental, | 74.7 | 85.0 | 66.7 | 72.9 | 64.8 | 72.8 | 8.0 | 64.8 | 85.0 |
| 4 U | | | | | | | | | |
| Linearized, | 69.5 | 80.9 | 64.9 | 73.1 | 59.0 | 69.5 | 8.2 | 59.0 | 80.9 |
| 4 U | | | | | | | | | |

Results for plasmid versus linear DNA indicates that enzyme processivity, which is expected to differ between plasmid and linear DNA, modestly affects the CpG site specific methylation levels. Methylated plasmid DNAs either have high or low CpG site specific methylation levels, while methylated linearized DNAs have primarily intermediate CpG site specific methylation levels.

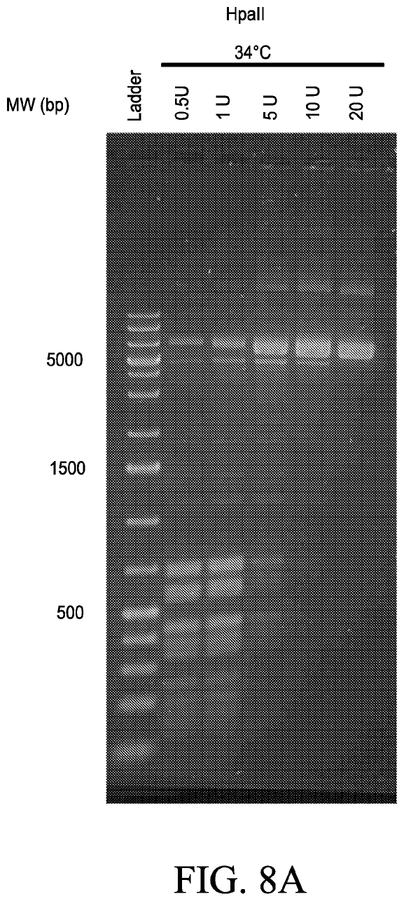

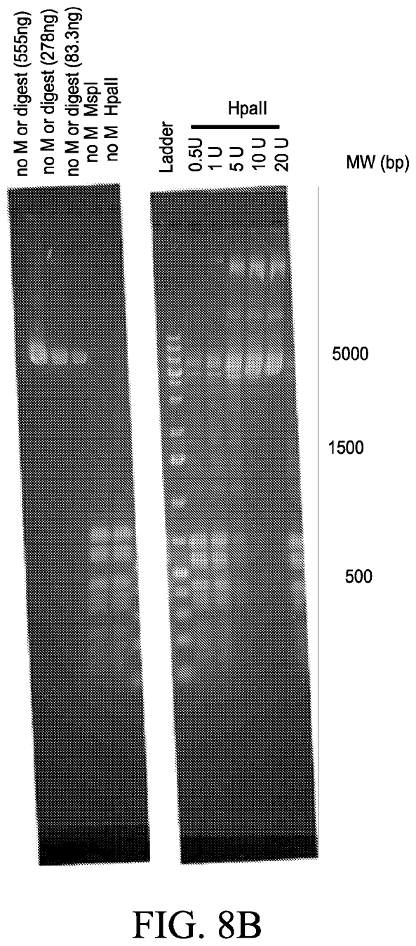

FIGS. 8A and 8B show results from additional reaction samples in which pSV40-sGAD55 source DNA was incubated with the indicated amounts of M.SssI methyltransferase at 34° C. for 1 hour. The results show that, like 20U, 10U of methylation enzyme resulted in nearly a 100% fractional methylation level, which is more than is often desired even for extensively enzymatically methylated DNA. To obtain a more optimal extensive fractional methylation level, such as in a range from 30% and 60%, incubation with a range from 1U to 3U is likely optimal.

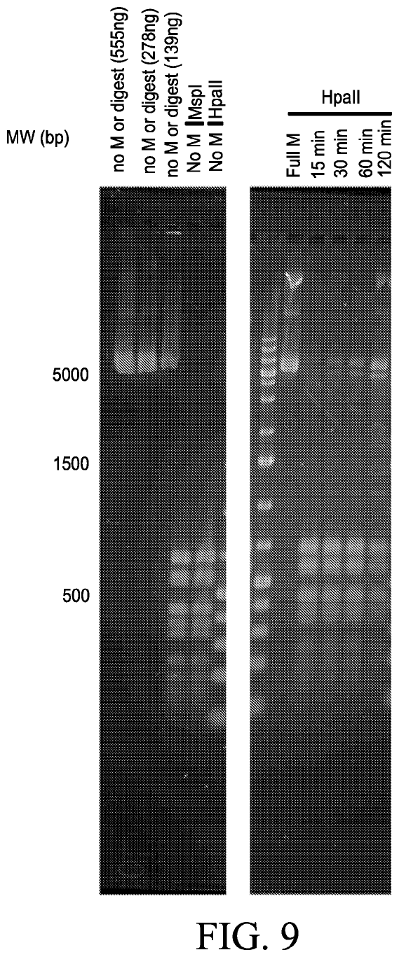

FIG. 9 shows the results for reaction samples in which pSV40-sGAD55 source DNA was incubated with 1U M.SssI methyltransferase at 34° C. for the indicated lengths of time. As the presence and/or intensity of bands in a range from about 600 bp to about 4000 bp indicates all types of methylation levels were sensitive to incubation time.

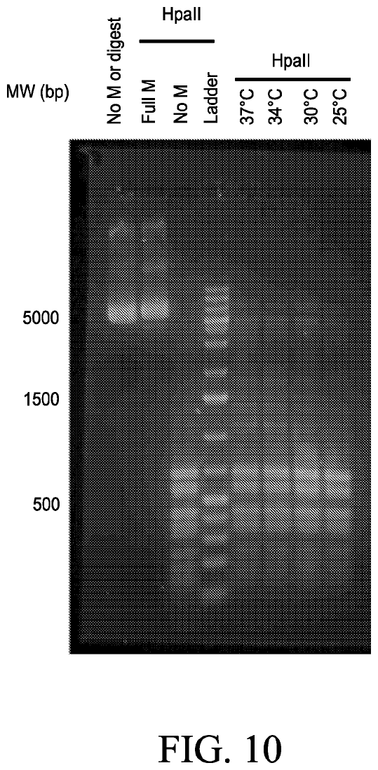

FIG. 10 shows the results for reaction samples in which pSV40-sGAD55 source DNA was incubated with 1U M.SssI methyltransferase at the indicated temperatures for 15 min.

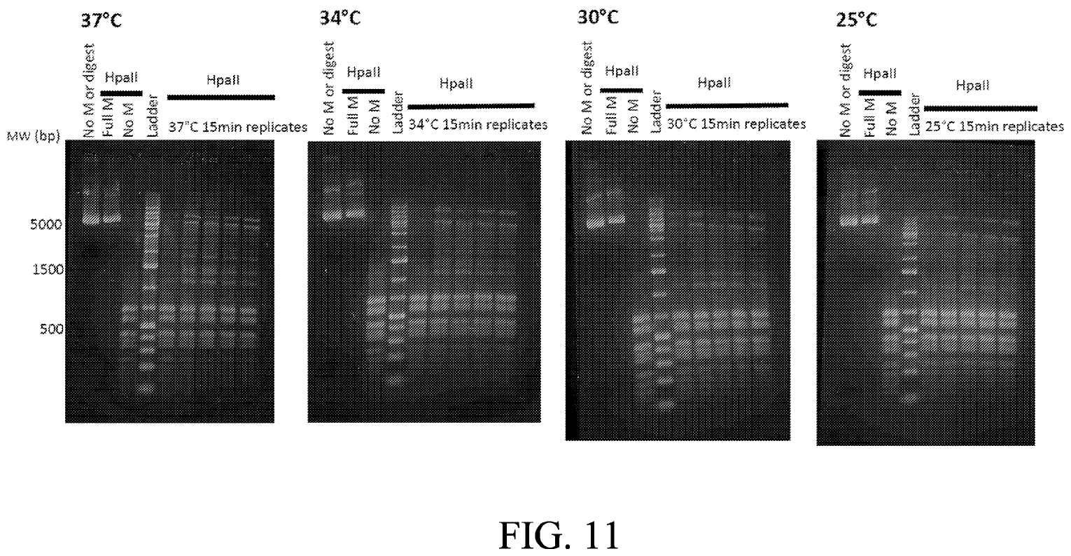

FIG. 11 shows the results for reaction samples in which pSV40-sGAD55 source DNA was incubated with 1 U M.SssI methyltransferase at the indicated temperatures for a time of 5 minutes. Five independent replicate samples were prepared for each temperature in FIG. 11. The results indicate that all types of methylation levels are somewhat sensitive to incubation temperature, but do not exhibit substantial changes in response to temperature. Results (not shown) obtained with bacterial methylation over these same temperature ranges exhibit substantially greater variation in all types of methylation levels.

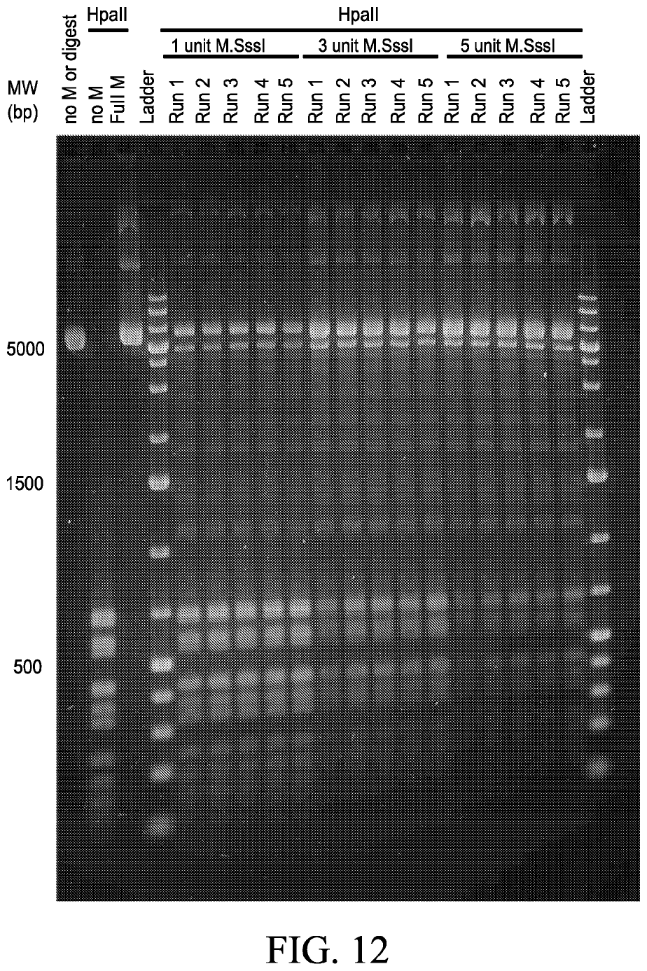

FIG. 12 shows results for reaction samples in which pSV40-sGAD55 source DNA was incubated with the indicated amounts of M. SssI methyltransferase at 34° C. for 60 minutes. Five separate samples were prepared for each enzyme concentration. The similarity in band patterns and intensities across samples prepared with the same enzyme concentration indicates that enzymatic methylation yields highly reproducible results. The lack of a substantially 100% fractional methylation level also further indicates that in a range from 1U to 3U may be optimal to achieve a fractional methylation level in a range of 30% and 60%.

The results of FIGS. 4-12 also demonstrate that similar methylation patterns were obtained in all tested reaction conditions.

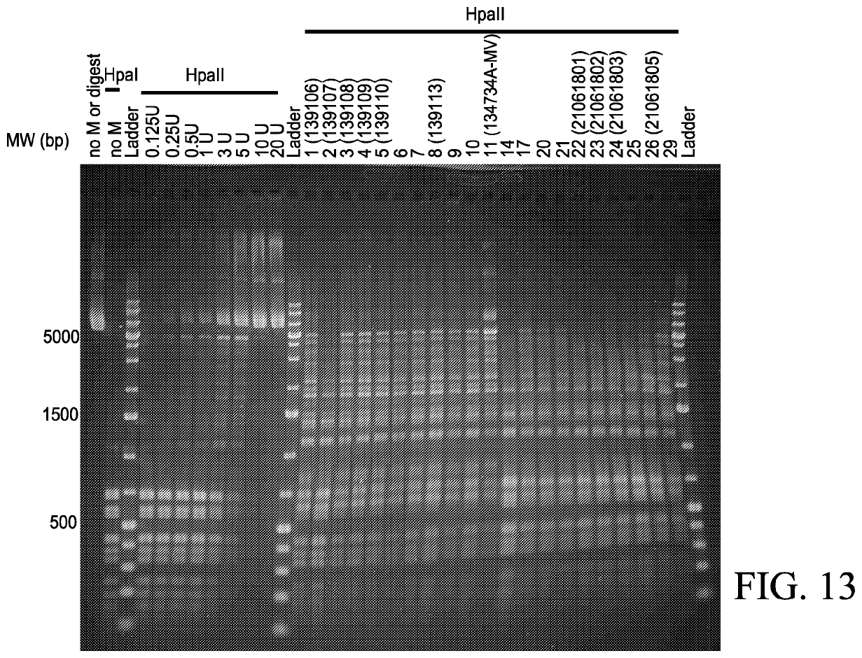

FIG. 13 shows results from reaction samples in which pSV40-sGAD55 source DNA was incubated with the indicated amounts of M. SssI methyltransferase at 34° C. temperature for 60 minutes, or in which source DNA was bacterially methylated using bacteria expressing M.SssI methyltransferase. The results, particularly the similar band sizes and intensities obtained using a range from 1U to 5U of M.SssI methyltransferase indicate that methylation patterns and all types of methylation levels can be obtained using enzymatic methylation that are similar to those obtained using bacterial methylation. Bacterial methylation resulted in brighter intermediate-sized bands, which may reflect M.SssI methyltransferase employing a more processive mode of action in enzymatic methylation under these conditions and a more distributive mode of action in bacterial methylation, as well as and other in vivo factors that affect the bacterial methylation samples.

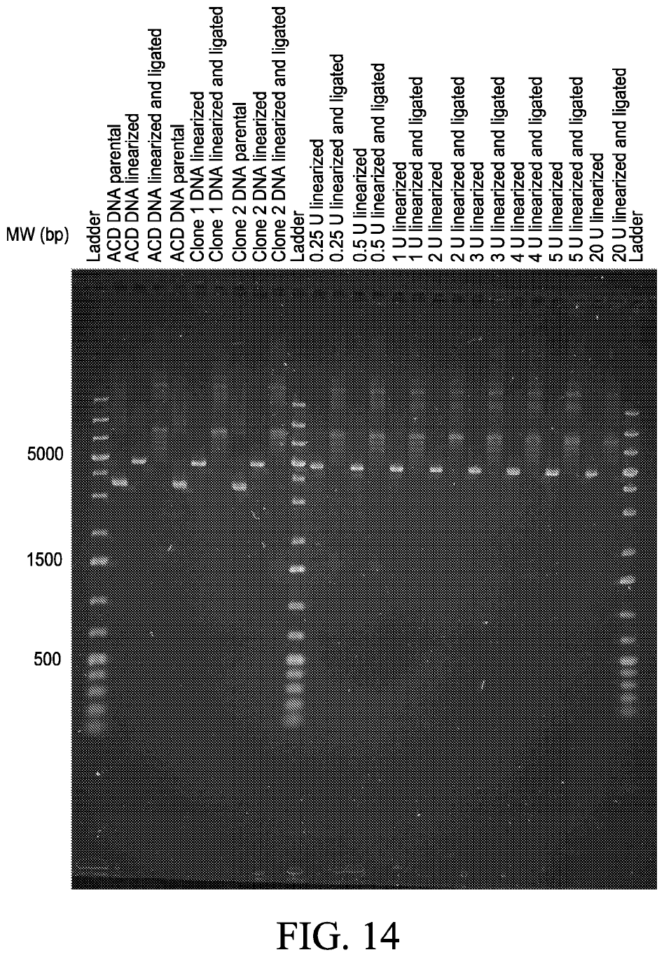

FIG. 14 shows that linearized enzymatically methylated DNA can be successfully ligated to reform a plasmid. T4 ligase was used for relegation of linearized enzymatically methylated DNA. Clone 1 and clone 2 are plasmid DNA from different bacterial clones to confirm consistency across sources.

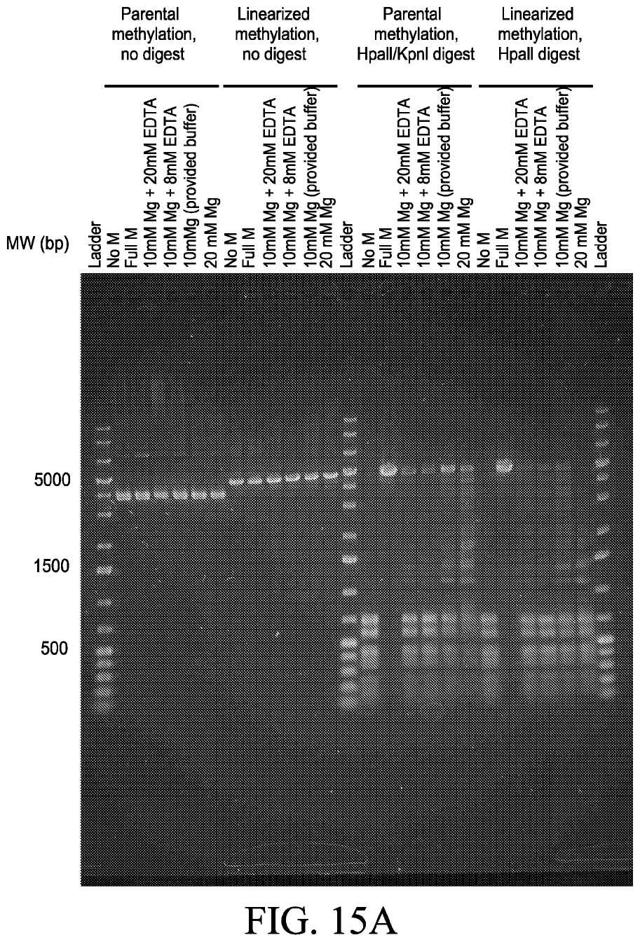

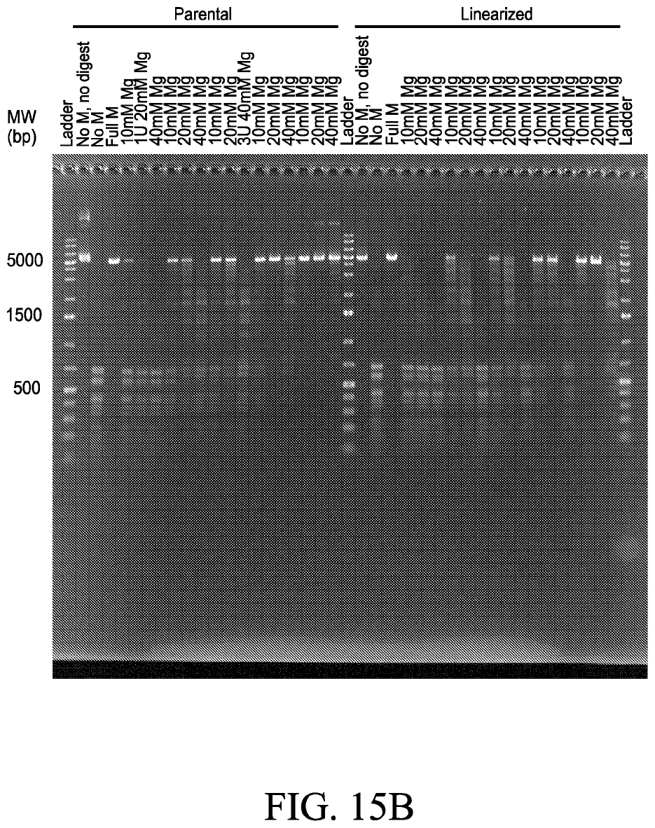

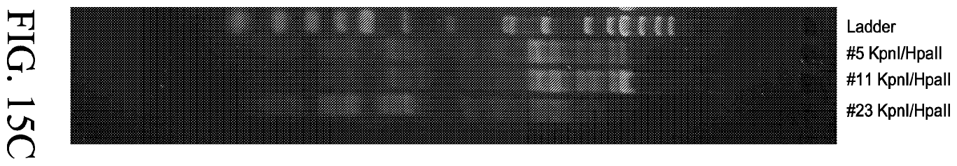

Test samples were prepared as above, but with 20 mM magnesium acetate, or in the standard 10 mM magnesium acetate buffer, but with 20 mM or 8 mM EDTA added to remove magnesium ion from the buffer. Results are presented in FIGS. 15A and 15B and show that the concentration of magnesium ion during enzymatic methylation affects methylation patterns and all types of methylation levels. Increasing amounts of magnesium ion changed the methylation pattern to one more consistent with distributive enzymatic activity and decreased the intensity of the methylated band corresponding to a fractional methylation level of 100%. 40 mM concentrations of magnesium ion caused a decrease in all types of methylation levels. Effects of magnesium ion varied depending on amount of enzyme present in the reaction sample, with detectable differences between 1-3U samples and 4-5U samples.

In addition, the data in FIG. 15B shows that methylation levels and patterns similar to that obtained using bacterial methylation were obtained by enzymatic methylation (shown in FIG. 15C). In particular, the #11 sample, which had 39.7% bacterial methylation, was similar in band patterns and intensity to the plasmid (parental) 2U 20 mM Mg, 3U 20 mM Mg, 4U 40 mM Mg samples and the linear 2U 10 mM Mg, 3U 20 mM Mg samples. In addition, the #5 sample, which had 29.0% bacterial methylation, was similar in band patterns and intensity to the plasmid (parental) 3U 40 mM Mg sample and the linear 2U 20 mM sample. Sample #23 has a level of bacterial methylation of all types lower than methylation levels observed in the enzymatically methylated samples.

The effects of magnesium concentration on enzymatic methylation and also the comparability of enzymatic methylation to bacterial methylation were also confirmed using pyrosequencing, the results of which are presented in Tables 3 and 4. Pyrosequencing results presented in Table 2, in which samples had 10 mM magnesium ion are also useful for comparison.

TABLE 3
Pyrosequencing Results For Various Concentrations
of Magnesium Ion (In Vitro Methylation)

| Sample | Methylation Sites | | | | | Overall Region | | | |
|---|---|---|---|---|---|---|---|---|---|
| ID | CpG#1 | Cpg#2 | CpG#3 | CpG#4 | CpG#5 | Mean | St. Dev. | Min. | Max. |
| Parental, | 29.5 | 43.8 | 26.3 | 31.1 | 30.2 | 32.2 | 6.7 | 26.3 | 43.8 |
| 2 U, | | | | | | | | | |
| 10 mM | | | | | | | | | |
| Mg | | | | | | | | | |
| Parental, | 25.0 | 56.3 | 26.2 | 33.8 | 44.3 | 37.1 | 13.2 | 25.0 | 56.3 |
| 2 U, | | | | | | | | | |
| 20 mM | | | | | | | | | |
| Mg | | | | | | | | | |
| Linearized, | 24.6 | 37.2 | 24.1 | 26.0 | 26.1 | 27.6 | 5.4 | 24.1 | 37.2 |
| 2 U, | | | | | | | | | |
| 10 mM | | | | | | | | | |
| Mg | | | | | | | | | |
| Linearized, | 13.2 | 29.0 | 12.5 | 16.8 | 17.9 | 17.9 | 6.6 | 12.5 | 29.0 |
| 2 U, | | | | | | | | | |
| 20 mM | | | | | | | | | |
| Mg | | | | | | | | | |

TABLE 4
Pyrosequencing Results For Comparative Bacterial
Methylation Samples (In vivo Methylation)

| Sample | Methylation Sites | | | | | Overall Region | | | |
|---|---|---|---|---|---|---|---|---|---|
| ID | CpG#1 | Cpg#2 | CpG#3 | CpG#4 | CpG#5 | Mean | St. Dev. | Min. | Max. |
| #5 | 9.5 | 61.0 | 18.2 | 19.4 | 37.1 | 29.0 | 20.5 | 9.5 | 61.0 |
| m[SV40- | | | | | | | | | |
| sGAD]- | | | | | | | | | |
| BLa30 | | | | | | | | | |
| #11 | 21.1 | 67.9 | 34.3 | 27.5 | 47.6 | 39.7 | 18.6 | 21.1 | 67.9 |
| m[SV40- | | | | | | | | | |
| sGAD]- | | | | | | | | | |
| BLa30 | | | | | | | | | |
| #14 | 2.8 | 25.5 | 5.8 | 5.8 | 12.5 | 10.5 | 9.1 | 2.8 | 25.5 |
| m[SV40- | | | | | | | | | |
| sGAD]- | | | | | | | | | |
| BLa34 | | | | | | | | | |
| #23 | 4.2 | 33.6 | 8.4 | 7.8 | 18.3 | 14.5 | 11.9 | 4.2 | 33.6 |
| m[SV40- | | | | | | | | | |
| sGAD]- | | | | | | | | | |
| BLa34GP | | | | | | | | | |

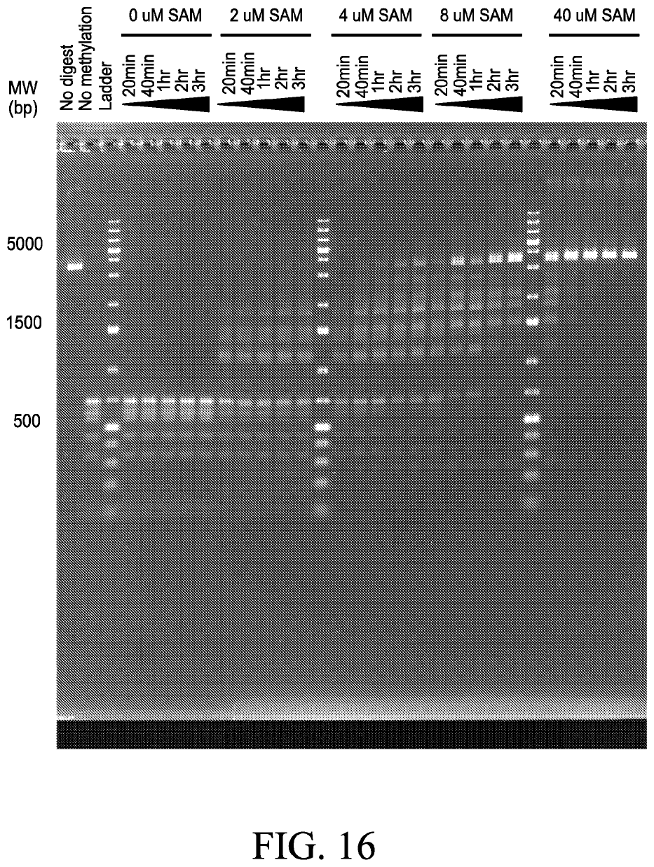

FIG. 16 shows results from reaction samples in which pSV40-sGAD55 source DNA was incubated with 4U M.SssI methyltransferase at 34° C. temperature for the indicated amount of time before quenching by incubation for 20 minutes at 65° C. The reaction samples included the indicated varying amounts of SAM. Additional magnesium chloride was also added. Nearly complete methylation was achieved with 40 μM SAM for incubation times of over 1 hour. However, lower amounts of SAM provided varying degrees of enzymatic methylation, demonstrating the methylation may also be controlled by limiting the concentration of SAM. Furthermore, even very low amounts of SAM, such as the 2 μM concentration, were still sufficient to allow some enzymatic methylation, even with only 20 minutes incubation time.

Changes can be made to the embodiments in light of the above-detailed description. For example, aspects of the embodiments can be modified, if necessary to employ concepts of the various patents, applications and publications incorporated by reference herein to provide yet further embodiments. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

References cited in Section 4 of Kurdyukov and Bullock:
High-Throughput DNA Methylation Profiling with VeraCode® Technology, <<http://www.illumina.com/content/dam/illumina-marketing/documents/products/datasheets/datasheet_veracode_methylation.pdf>> (2015).
Methprimer, <http://www.urogene.org/methprimer>> (2015).
Kurdyukov S., Mathesius U., Nolan K. E., Sheahan M. B., Goffard N., Carroll B. J., Rose R. J., “The 2ha line of Medicago truncatula has characteristics of an epigenetic mutant that is weakly ethylene insensitive,” Bmc Plant Biol. 14:174, (2014).
Mahapatra S., Klee E. W., Young C. Y., Sun Z., Jimenez R. E., Klee G. G., Tindall D. J., Donkena K. V., “Global methylation profiling for risk prediction of prostate cancer,” Clin. Cancer Res. 18:2882-2895, (2012).
Herman J. G., Graff J. R., Myohanen S., Nelkin B. D., Baylin S. B., “Methylation-specific pcr: A novel pcr assay for methylation status of cpg islands,” Proc. Natl. Acad. Sci. USA. 93:9821-9826 (1996).
Wojdacz T. K., Dobrovic A., Hansen L. L., “Methylation-sensitive high-resolution melting,” Nat. Protoc. 3:1903-1908 (2008).
Castellanos-Rizaldos E., Milbury C. A., Karatza E., Chen C. C., Makrigiorgos G. M., Merewood A., “Cold-pcr amplification of bisulfite-converted DNA allows the enrichment and sequencing of rare un-methylated genomic regions,” PLOS ONE. 9:3 (2014)
Yokoyama S., Kitamoto S., Yamada N., Houjou I., Sugai T., Nakamura S., Arisaka Y., Takaori K., Higashi M., Yonezawa S., “The application of methylation specific electrophoresis (mse) to DNA methylation analysis of the 5′ cpg island of mucin in cancer cells,” BMC Cancer. 12:67 (2012)

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 63/238,726, filed on Aug. 30, 2021, U.S. Provisional Patent Application No. 63/240,341, filed on Sep. 2, 2021, and U.S. Provisional Patent Application No. 63/247,714, filed on Sep. 23, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of enzymatically methylating a DNA, the method comprising:
combining a source DNA encoding a pro-apoptotic protein, a determinant protein, or a functional fragment thereof, an extracellular methylation enzyme and an enzymatic substrate in an amount sufficient to allow methylation of at least one CpG site on the source DNA; and
incubating the reaction sample at a temperature and for a time sufficient to obtain an enzymatically methylated DNA having a CpG site specific attenuating or stimulating methylation level at least one CpG site, a mean whole DNA methylation level, or a fractional methylation level, or a specific methylation pattern.

2. (canceled)

3. The method of claim 1, wherein the source DNA has a mean whole DNA methylation level of less than about 3%.

4. The method of claim 1, wherein the source DNA is produced in a host cell that has a methyltransferase gene, is produced in a host cell that lacks a methyltransferase gene, or is artificially synthesized.

5-6. (canceled)

7. The method of claim 1, wherein the source DNA is linear or a plasmid.

8. The method of claim 1, further comprising a second methylation enzyme.

9. The method of claim 1, wherein at least one methylation enzyme is a M.SssI methyltransferase, M.MpeI methyltransferase, an AluI methyltransferase, a HaeIII methyltransferase, a HhaI methyltransferase, a HpaII methyltransferase, a MspI methyltransferase, a BamHI methyltransferase, a Dam methyltransferase, an EcoGII methyltransferase, an EcoRI methyltransferase, a GpC methyltransferase (M.CviPI), a MspI methyltransferase, a TaqI methyltransferase, DNMT1, DNMT2, DNMT3a, or DNMT3b.

10. The method of claim 1, wherein at least one methylation enzyme is present in the reaction sample in an amount of at least about 0.1U.

11. The method of claim 1, where incubation is for a period of time in a range from about 1 minute to about 24 hours.

12. The method of claim 1, wherein the incubation is at a temperature in arrange a range from about 25° C. to about 45° C.

13. The method of claim 1, further comprising combining magnesium ion in the reaction sample.

14. The method of claim 1, further comprising treating the source DNA prior to combining to remove one or more associated material that affects DNA accessibility or to cause a conformational change of the source DNA.

15. The method of claim 1, further comprising treating the enzymatically methylated DNA to alter its structure.

16-17. (canceled)

18. The method of claim 1, wherein the enzymatically methylated DNA is pSV40-sGAD55, pSV40-hBAX-BLa, or pSV40-sGAD55+hBAX-BLa, or has a polynucleotide sequence of SEQ ID NO:1 or SEQ ID NO: 2.

19. The method of claim 1, wherein the determinant protein is an allergen, an autoantigen, a cancer antigen, a donor antigen, a sequestered tissue specific antigen, or a functional fragment thereof.

20. The method of claim 1, wherein the source DNA further encodes a tolerance inducing protein or a functional fragment thereof.

21. The method of claim 1, further comprising demethylating the source DNA prior to combining the source DNA with the extracellular methylation enzyme.

22. An enzymatically methylated DNA prepared according to claim 1, wherein the enzymatically methylated DNA encodes a pro-apoptotic protein or a functional fragment thereof or a cancer antigen and has a stimulating methylation level, or wherein the enzymatically methylated DNA encodes an allergen, and autoantigen, a donor antigen, a sequestered tissue specific antigen, or a functional fragment thereof and has an attenuating methylation level.

23-24. (canceled)

25. A DNA having SEQ ID NO:1 or SEQ ID NO: 2.

26. A pharmaceutical composition comprising an enzymatically methylated DNA of claim 22 and a pharmaceutically acceptable carrier.

27. A method of treating an allergy, an autoimmune disease, cancer, or transplant rejection in a patient, the method comprising administering to the patient a therapeutically effective amount of an enzymatically methylated DNA of claim 22.
